(19) United States
(12) Patent Application Publication
(10) Pub. No.: US 2025/0115916 A1
(43) Pub. Date: Apr. 10, 2025

(54) MODULATION OF SUV39H1 EXPRESSION BY RNAS

(71) Applicants: MNEMO THERAPEUTICS, Paris (FR); INSTITUT CURIE, Paris (FR); INSERM, Paris (FR)

(72) Inventors: Sebastian AMIGORENA, Paris (FR); Michael SAITAKIS, Paris (FR); Sheila LOPEZ-COBO, Paris (FR); Jaime Rodrigo FUENTEALBA, Paris (FR)

(21) Appl. No.: 18/729,783

(22) PCT Filed: Jan. 23, 2023
(86) PCT No.: PCT/EP2023/051593

§ 371 Date: Jul. 17, 2024

Related U.S. Application Data
(60) Provisional application No. 63/301,845, filed on Jan. 21, 2022.

(51) Int. Cl. C12N 15/113; A61K 35/17; A61K 40/31; A61K 40/32; C07K 16/28

(52) U.S. Cl. CPC C12N 15/1137; A61K 35/17; A61K 40/31; A61K 40/32; C07K 16/2818; C07K 16/2827; A61K 2239/13; A61K 2239/21; A61K 2239/22; C12N 2310/14; C12N 2310/531; C12Y 201/01

(57) ABSTRACT

The invention provides compositions and methods for modulating expression of SUV39H1 using inhibitory or activating polynucleotides based on the sequence of a long noncoding RNA or of a short hairpin RNA (shRNA).

### FIELD OF THE INVENTION

The invention relates to compositions and methods for modulating expression of SUV39H1 using RNAs.

### BACKGROUND OF THE INVENTION

Long noncoding RNAs (lncRNAs) are regulators of gene expression and are involved in many cellular processes. Their expression has been found to be particularly tissue-specific or developmental stage-specific (Ahmad et al, 2021).

Small hairpin RNAs (shRNA) are sequences of RNA, typically about 80 base pairs in length, that include a region of internal hybridization that creates a hairpin structure. shRNA molecules are processed within the cell to form siRNA which in turn knock down gene expression. The benefit of shRNA is that they can be incorporated into plasmid vectors and integrated into genomic DNA for long-term or stable expression, and thus long knockdown of the target mRNA.

Histone methyltransferases promote chromatin rearrangements into euchromatin (open, actively transcribed chromatin) and heterochromatin (closed, inactive chromatin) via their post-translational modifications of histone molecules at the nucleosomes. Histone methyltransferases actively control gene expression and cell fate. SUV39H1 was one of the first methyltransferases identified (Aagaard et al, 1999). It has been shown to be involved in a multitude of developmental processes, particularly in the immune system (Rao et al, 2017; Nicetto & Zaret, 2019; Allan et al, 2012; Pace et al, 2018).

Pace et al., 2018 and Int'l Patent Pub. No. WO 2018/234370 disclose that inhibition of the expression of SUV39H1 in T cells and NK cells enhances their memory potential and increases survival capacity.

### SUMMARY OF THE INVENTION

The disclosure provides compositions and methods for modulating expression of SUV39H1 using RNAs.

The invention relates to a cell including a first nucleic acid that is a heterologous nucleic acid that expresses an RNA including the nucleobase sequence of [SEQ ID NO: 1] (lncRNA AF196970.3) or a fragment thereof capable of inhibiting expression of SUV39H1 in the cell; a heterologous nucleic acid including the nucleotide sequence of [SEQ ID NO: 5] (genomic sequence encoding AF196970.3 (ENSG00000232828)) or a fragment thereof that expresses an RNA capable of inhibiting expression of SUV39H1 in the cell; a nucleic acid that expresses an RNA including the nucleobase sequence of [SEQ ID NO: 1] (lncRNA AF196970.3) or a fragment thereof capable of inhibiting expression of SUV39H1 in the cell, said nucleic acid operatively linked to a heterologous expression control sequence (e.g., promoter, enhancer, other regulatory sequence); or a nucleic acid operatively linked to a heterologous expression control sequence including the nucleotide sequence of [SEQ ID NO: 5] (genomic sequence encoding AF196970.3 (ENSG00000232828)) or a fragment thereof that expresses an RNA capable of inhibiting expression of SUV39H1 in the cell, said nucleic acid operatively linked to a heterologous expression control sequence. The first nucleic acid may include the nucleotide sequence of any of SEQ ID NO: 5-9 or a fragment thereof. The first nucleic acid may express an RNA including the nucleobase sequence of [SEQ ID NO: 2] (lncRNA AF196970.3 exon 1) or a fragment thereof capable of inhibiting expression of SUV39H1 in the cell. The first nucleic acid may express an RNA including the nucleobase sequence of [SEQ ID NO:3] (lncRNA AF196970.3 exon 2) or a fragment thereof capable of inhibiting expression of SUV39H1 in the cell. The first nucleic acid may express an RNA including the nucleobase sequence of [SEQ ID NO: 4] (lncRNA AF196970.3 exon3) or a fragment thereof capable of inhibiting expression of SUV39H1 in the cell. In any of the foregoing embodiments, the first nucleic acid may express an RNA at least about 12-50, 50-100, 100-150, 150-200, 200-250, 250-300, 300-350, 350-400, 400-450, 450-500, 500-550, 550-600, 600-650, 650-700, 700-750, 750-800, 800-850, 850-900, 250-750, 500-750 or more bases in length.

The invention also relates to a cell including a nucleic acid that is a heterologous nucleic acid that expresses an RNA including the nucleotide sequence of any one of SEQ ID NO: 13-17 or SEQ ID 26-30 or a fragment or derivative thereof capable of inhibiting expression of SUV39H1 in the cell; or a fragment thereof that expresses an RNA capable of inhibiting expression of SUV39H1 in the cell; a nucleic acid that expresses an RNA including the nucleotide sequence of any one of SEQ ID 13-17 or SEQ ID NO:26-30 or a fragment thereof capable of inhibiting expression of SUV39H1 in the cell, said nucleic acid being typically operatively linked to a heterologous expression control sequence (e.g., promoter, enhancer, other regulatory sequence). The nucleic acid may include the nucleotide sequence of any of SEQ ID NO: 13-17 or 26-30 or a fragment thereof.

The invention also relates to a cell including a heterologous polynucleotide including the nucleotide sequence of any one of SEQ ID NO: 32-36 and 45-49 or a fragment thereof capable of inhibiting expression of SUV39H1 in the cell. In some embodiments, said nucleic acid can be operatively linked to a heterologous expression control sequence (e.g., promoter, enhancer, other regulatory sequence).

The cells may be modified immune cells.

The cells may be any of the following cell types: a T cell, a CD4+ T cell, a CD8+ T cell, a CD4+ and CD8+ T cell, a NK cell, a Treg cell, a Tm cell, a memory stem T cell (TSCM), a TCM cell, a TEM cell, a monocyte, a dendritic cell, or a macrophage a T cell progenitor, an NK cell progenitor, a pluripotent stem cell, an induced pluripotent stem cell (iPSC), a hematopoietic stem cell (HSC), an adipose derived stem cell (ADSC), a pluripotent stem cell of myeloid or lymphoid lineage.

The cells may include one or more engineered receptors, two or more, or three or more.

The cells may include a second heterologous nucleic acid that expresses one or more engineered receptors. The engineered receptor may include a) an extracellular antigen-binding domain that specifically binds an antigen, optionally including an antibody heavy chain variable region and/or an antibody light chain variable region, and may optionally be bispecific or trispecific; b) a transmembrane domain, optionally including a fragment of transmembrane domain of alpha, beta or zeta chain of the T-cell receptor, CD28, CD3 epsilon, CD45, CD4, CD5, CD8, CD9, CD16, CD22, CD33, CD37, CD64, CD80, CD86, CD34, CD137, or CD154, NKG2D, OX40, ICOS, 2B4, DAP10, DAP12, CD40; c) optionally one or more co-stimulatory domains from 4-1BB, CD28, ICOS, OX40, DAP10 or DAP12, 2B4, CD40, FCER1G; and d) an intracellular signaling domain including an intracellular signaling domain from CD3zeta, FcR gamma, FcR beta, CD3 gamma, CD3 delta, CD3 epsilon, CDS, CD22, CD79a, CD79b, or CD66d, 2B4, or any fragment thereof. The engineered receptor may be a chimeric antigen receptor (CAR) including an extracellular antigen-binding domain, optionally an scFv, a transmembrane domain, optionally from CD28, CD8 or CD3-zeta, one or more co-stimulatory domains, optionally from 4-1 BB, CD28, ICOS, OX40 or DAP10, and an intracellular signaling domain from CD3zeta, optionally in which ITAM2 and ITAM3 have been inactivated.

The engineered receptor may also be a modified TCR.

The modified TCR may comprise (a) an extracellular domain that comprises antigen-binding fragments or CDRs of an antibody, preferably all three CDRs of a heavy chain variable region (VH) and/or a light chain variable region (VL), and (b) a native or variant constant region of an alpha, beta, gamma or delta chain. For example, the modified TCR may comprise one or more heterologous polypeptides, for example, (a) VH of an antibody or a fragment or variant having at least 90% sequence identity thereto, fused to TRBC1 (typically a human or murine TRBC1) or TRBC2 (typically a human or murine TRBC2), or a fragment or variant of TRBC1 or TRBC2 having at least 90% sequence identity thereto, and (b) a VL of an antibody or a fragment or variant having at least 90% sequence identity thereto fused to TRAC (human or murine TRAC sequence), or a fragment or variant of TRAC having at least 90% sequence identity thereto. The modified TCR may optionally further comprise a native or variant CD3zeta polypeptide, e.g. a modified CD3zeta polypeptide in which one or two of the ITAM domains (e.g. ITAM2 and ITAM3) have been deleted.

Recombinant HLA-independent (or non-HLA restricted) modified TCR (referred to as “HI-TCRs”) that bind to an antigen of interest in an HLA-independent manner are typically described in International Application No. WO 2019/157454. Such HI-TCRs comprise an antigen binding chain that comprises: (a) a heterologous antigen-binding domain that binds to an antigen in an HLA-independent manner, for example, an antigen-binding fragment of an immunoglobulin variable region; and (b) a constant domain that is capable of associating with (and consequently activating) a CD3zeta polypeptide. Preferably, the antigen-binding domain or fragment thereof comprises: (i) a heavy chain variable region (VH) of an antibody and/or (ii) a light chain variable region (VL) of an antibody. The constant domain of the TCR is, for example, a native or modified TRAC polypeptide (typically human or murine TRAC sequence), or a native or modified TRBC polypeptide (typically human or murine TRBC sequence). The constant domain of the TCR is, for example, a (human or murine) native TCR constant domain (alpha or beta) or fragment thereof. Unlike chimeric antigen receptors, which typically themselves comprise an intracellular signaling domain, the HI-TCR does not directly produce an activating signal; instead, the antigen-binding chain associates with and consequently activates a CD3zeta polypeptide. The immune cells comprising the recombinant TCR provide superior activity when the antigen has a low density (typically a median density) on the cell surface of less than about 10,000 molecules per cell, e.g. less than about 5,000, 4,000, 3,000, 2,000, 1,000, 500, 250 or 100 molecules per cell.

The engineered receptor may thus typically be a modified TCR including a first antigen-binding chain including an antigen-binding fragment of a heavy chain variable region (VH) of an antibody; and a second antigen-binding chain including an antigen-binding fragment of a light chain variable region (VL) of an antibody; wherein the first and second antigen-binding chains each includes a TRAC polypeptide or a TRBC polypeptide, optionally wherein at least one of the TRAC polypeptide and the TRBC polypeptide is endogenous, and optionally wherein one or both of the endogenous TRAC and TRBC polypeptides is inactivated.

The cell may further comprise a chimeric co-stimulatory receptor that comprises (a) an extracellular and transmembrane domain of CD86, 41BBL, CD275, CD40L, OX40L, PD-1, TIGIT, 2B4, or NRP1, or fragment or variant thereof, and (b) an intracellular co-stimulatory molecule of CD28, 4-1BB, OX40, ICOS, CD27, CD40, or CD2, or fragment or variant thereof. In some embodiments, the chimeric co-stimulatory receptor includes (a) an extracellular domain of a co-stimulatory ligand, optionally from CD80, (b) a transmembrane domain, optionally from CD80, and (c) an intracellular domain of a co-stimulatory molecule, optionally CD28, 4-1BB, OX40, ICOS, DAP10, CD27, CD40, NKGD2, or CD2, preferably 4-1BB.

The extracellular antigen-binding domain of the cell may bind an antigen with a KD affinity of about 1×10⁻⁷ or less, about 5×10⁻⁸ or less, about 1×10⁻⁸ or less, about 5×10⁻⁹ or less, about 1×10⁻⁹ or less, about 5×10⁻¹⁰ or less, about 1×10⁻¹⁰ or less, about 5×10⁻¹¹ or less, about 1×10⁻¹¹ or less, about 5×10⁻¹² or less, or about 1×10⁻¹² or less (lower numbers indicating higher affinity). The antigen bound by the extracellular antigen-binding domain may have a low density on the cell surface, of less than about 10,000, or less than about 5,000, or less than about 2,000 molecules per cell.

The extracellular antigen-binding domain may bind to antigens, for example,
orphan tyrosine kinase receptor ROR1, tEGFR, Her2, p95HER2, LI-CAM, CD19, CD20, CD22, mesothelin, CEA, Claudin 18.2, hepatitis B surface antigen, anti-folate receptor, CD23, CD24, CD30, CD33, CD38, CD44, CD70, EGFR, EGP-2, EGP-4, EPHa2, ErbB2, 3, or 4, FBP, FcRH5, fetal acetylcholine e receptor, GD2, GD3, HMW-MAA, IL-22R-alpha, IL-13R-alpha2, kdr, kappa light chain, BCMA, Lewis Y, MAGE-A1, mesothelin, MUC1, MUC16, PSCA, NKG2D Ligands, NY-ESO-1, MART-1, gp100, oncofetal antigen, TAG72, VEGF-R2, carcinoembryonic antigen (CEA), prostate specific antigen (PSMA), estrogen receptor, progesterone receptor, ephrinB2, CD 123, CS-1, c-Met, GD-2, MAGE A3, CE7, or Wilms Tumor 1 (WT-1). In some embodiments, the antigen may be any of the tumor neoantigenic peptides disclosed in Int'l Pat. Pub. No. WO 2021/043804, WO 2022/189620, WO 2022/189626, WO 2022/189636 incorporated by reference herein in its entirety.

The cell may include two engineered antigen receptors that each bind different antigens.

The cell may be autologous or allogeneic.

SUV39H1 expression may be reduced or inhibited in the cell by at least about 50%, 60%, 70%, 75%, 80%, 85%, 90% or 95%.

The cell may initially be isolated from a subject suffering from a cancer, or at risk of suffering from a cancer.

The invention also relates to modified oligonucleotides including a nucleobase sequence from any of [SEQ ID NO: 1-4] at least about 12 bases in length, wherein the modified oligonucleotide includes one or more of a modified backbone linkage, a modified sugar moiety, a modified phosphate moiety, a modified nucleobase, or a chemically conjugated moiety. The modified oligonucleotide may be at least about 12-50, 50-75 or 50-100 bases in length. The modified oligonucleotide may include a modified backbone linkage that includes phosphorothioate, phosphonoacetate, thiophosphonoacetate, methylphosphonate, boranophosphate, or phosphorodithioate moities. The modified oligonucleotide may include a modified sugar that is modified to replace the 2′ OH-group by another group optionally, H, —OR, —R (wherein R can be, such as, alkyl, cycloalkyl, aryl, aralkyl, heteroaryl or sugar), halo, —F, —Br, —Cl or —I, —SH, —SR (wherein R can be, such as, alkyl, cycloalkyl, aryl, aralkyl, heteroaryl or sugar), F-arabino, F-arabino, amino (wherein amino can be, such as, NH2; alkylamino, dialkylamino, heterocyclyl, arylamino, diarylamino, heteroarylamino, diheteroarylamino, or amino acid); or cyano (—CN); optionally 2′-O-methoxy, 2′-O-methoxyethyl modifications, 2′-fluoro, 2′-deoxy, or combinations thereof. The modified oligonucleotide may include a modified nucleobase that includes one or more 5-methylcytosines, modified uridines, such as, 5-(2-amino) propyl uridine, and 5-bromo uridine, modified adenosines and guanosines, such as, with modifications at the 8-position, such as, 8-bromo guanosine, a deaza nucleotide, such as, 7-deaza-adenosine, or O- and N-alkylated nucleotides, such as, N6-methyl adenosine, or a modified nucleotide which is multicyclic (such as, tricyclo; and “unlocked” forms, such as glycol nucleic acid (GNA) (such as, R-GNA or S-GNA, where ribose is replaced by glycol units attached to phosphodiester bonds), or threose nucleic acid (TNA, where ribose is replaced with α-L-threofuranosyl-(3′→2′)).

The invention also relates to a nucleic acid operatively linked to a heterologous expression control sequence, said nucleic acid including (a) a nucleotide sequence that encodes or expresses an RNA including the nucleobase sequence of [SEQ ID NO: 1] (lncRNA AF196970.3) or the nucleotide sequence of any one of SEQ ID NO:13-17 and 26-30 or a fragment thereof capable of inhibiting expression of SUV39H1 in the cell, or (b) the nucleotide sequence of [SEQ ID NO: 5] (genomic sequence encoding AF196970.3 (ENSG00000232828)) of any one of SEQ ID NO:32-36 and 45-49, or a fragment thereof that expresses an RNA capable of inhibiting expression of SUV39H1 in the cell. The nucleic acid may include the nucleotide sequence of any of SEQ ID NO: 5-9, 32-36, 42-49 or a fragment or fragments thereof. The heterologous control sequence may be a constitutive, inducible, or tissue-specific promoter, optionally EF1α, CMV, SFFV, hPGK, RPBSA, or CAG.

The invention also relates to a vector including a nucleic acid as herein described and one or more additional expression control sequences. The vector a viral vector, optionally an adenovirus, adeno-associated virus (AAV), poxvirus, papillomavirus, lentivirus, retrovirus, herpes virus, foamivirus, or Semliki Forest virus vector, and including pseudotyped viruses.

The invention also relates to a delivery vehicle, optionally a liposome, lipid-containing complex, nanoparticle, gold particle, or polymer complex including the nucleic acid or vector described above.

The invention also relates to a method of producing the cells described herein including at least the steps of (a) introducing into the cell (i) a nucleic acid as described herein or (ii) a vector as described herein, and optionally (b) introducing into the cell a nucleic acid encoding an antigen-specific receptor.

The invention also relates to a method of producing a cell as described herein including at least the step of introducing into the cell a heterologous expression control sequence in a manner such that it is operatively linked to an endogenous nucleic acid that expresses an RNA including the nucleobase sequence of [SEQ ID NO: 1] (lncRNA AF196970.3), or the nucleotide sequence of any one of SEQ ID NO: 13-17, 26-30 or a fragment thereof capable of inhibiting expression of SUV39H1 in the cell, optionally a nucleic acid including the nucleotide sequence of [SEQ ID NO: 5] (genomic sequence encoding AF196970.3 (ENSG00000232828) or an allelic variant thereof or any one of SEQ ID NO:32-36 and 45-49.

The invention also relates to a method of using a cell as described herein to treat a disease including administering to a subject in need thereof an amount of the cells effective to treat the disease, optionally cancer, an infectious disease, an autoimmune disease, an inflammatory disease, or an allergic disease, wherein the cells express one or more antigen-specific receptors that bind an antigen associated with a disease.

The invention also relates to a method for treating a subject suffering from cancer including administering to said subject: (1) a cell as described herein; and (2) a second cancer therapeutic agent. The second cancer therapeutic agent may be an immune checkpoint modulator, cancer vaccine, chemotherapeutic or anti-angiogen.

The invention also relates to a method for treating a subject suffering from cancer including administering to said subject: (1) a cell as described herein, wherein the cell is a T cell, NK cell or T cell progenitor, including a genetically engineered antigen receptor wherein expression of the SUV39H1 gene is inhibited, and wherein inhibitions of the SUV39H1 gene results in enhanced anti-cancer activity of said immune cell; and (2) an immune checkpoint modulator. The immune checkpoint modulator may be an inhibitor of PD1, CTLA4, LAG3, BTLA, OX2R, TIM-3, TIGIT, LAIR-1, PGE2 receptor, EP2/4 adenosine receptor, or A2AR. The immune checkpoint modulator may be an anti-PD-1 inhibitor or anti-PDL-1 inhibitor.

### BRIEF DESCRIPTION OF THE DRAWINGS

FIG. 1 shows the SUV39H1 gene locus containing the gene ENSG00000232828 encoding for (expressing) lncRNA AF196970.3 in the genome browser of UCSC (genome-euro.ucsc.edu).

FIG. 2A shows the lncRNA AF196970.3 with its 3 exons, FIG. 2B shows the overlap in the genomic region and the shared nucleotides between lncRNA AF196970.3 exon 2 and SUV39H1 exon 3, and C) the overlap in the genomic region and the shared nucleotides between lncRNA AF196970.3 exon 3 and SUV39H1 exon 2.

FIG. 3A shows the bulk tissue gene expression for AF196970.3, FIG. 3B shows the bulk tissue gene expression for SUV39H1, FIG. 3C shows single-cell expression for AF196970.3 in different tissues, FIG. 3D shows single-cell expression for SUV39H1 in different tissues, and FIG. 3E shows the expression of transcript ENST00000416061.1 of AF196970.3 in different tissues.

FIG. 4A, FIG. 4B and FIG. 4C show the expression levels of AF196970.3 and SUV39H1 in single-cell RNAseq derived clusters from a glioma, a head-and-neck squamous cell carcinoma, and a hepatocellular carcinoma, respectively.

FIG. 5 shows plasmids maps representing Piggy Bac backbone with GFP-puromycin reporter and including lncRNA SUV39H1 exonic sequence with a CMV promoter (5A), or lncRNA SUV39H1 exonic sequence with a hPKG promoter (5B).

FIG. 6A shows schematic of the experimental procedure of Piggy Bac transfection in HEK293 FT cells using the two plasmids constructs of FIG. 5. FIG. 6B shows GFP expression levels by flow cytometry and comparison of non-transfected cells with cells transfected with either Piggy Bac construct empty or one of the lncRNA SUV39H1 Piggy Bac plasmid. FIG. 6C shows expression of SUV39H1 and actin by western blotting in cells. Quantification of SUV SUV39H1 protein level normalized against actin. FIG. 6D illustrates a schematic of the experimental procedure of lncRNA SUV39H1 expression in CD8+ T-cells.

FIG. 7A. shows a representation of shRNA target sequences on SUV39H1 gene. FIG. 7B shows the target and loop sequences of different shRNA targeting SUV39H1. FIG. 7C shows a plasmid map of the lentiviral construct used for the shRNA expression in cells using a U6 promoter and an EGFP reporter.

FIG. 8A illustrates the experimental procedure of shRNA expression in HEK293 FT cells. FIG. 8B shows GFP expression levels by flow cytometry: comparison of non-transduced cells with cells transduced with either scramble shRNA or one of the 5 shRNA against SUV39H1. FIG. 8C shows the expression of SUV39H1 and actin by western blotting in cells.

FIG. 9A shows illustrated the experimental procedure of shRNA expression in CD8+ T-cells. FIG. 9B shows the GFP expression levels by flow cytometry: comparison of non-transduced cells with cells transduced with either scramble shRNA or one of the 5 shRNA against SUV39H1. Results are shown for a representative donor. FIG. 9C shows the expression of SUV39H1 and actin by western blotting in cells. Control cells in the absence (Mock) or presence (gRNA SUV) of a SUV39H1-targeting gRNA. FIG. 9D shows the levels of tri-methylation of H3K9 by flow cytometry (geometric mean fluorescence intensity) in T cells. Results are shown for a representative donor. FIG. 9E-F show the expression by flow cytometry of the memory marker CD27 in T cells at day 7: comparison of scramble sequence against shRNA 1 for 3 donors (mean of duplicate). FIG. 9F shows the expression by flow cytometry of the memory marker CD27 in T cells at day 14: comparison of scramble sequence against shRNA 1 for 2 donors (mean of duplicate).

### DETAILED DESCRIPTION OF THE INVENTION

SUV39H1 is a H3K9-histone methyltransferase that plays a role in silencing memory and stem cell programs during the terminal differentiation of effector CD8+ T cells. Silencing of SUV39H1, in turn, has been shown to enhance long-term memory potential and to increase survival capacity. Human SUV39H1, see UniProt Accession No. 043463.

The present disclosure relates to the identification of the antisense lncRNA, AF196970.3, that has a silencing function against SUV39H1 in human cells, inhibiting SUV39H1 expression and thus lowering levels of SUV39H1 protein in the cell. AF196970.3 has an expression pattern very similar to the expression pattern of SUV39H1. It is detected in many different cell types with highest levels in, for example, in endothelial cells, fibroblasts and myocytes, similar to SUV39H1. AF196970.3 is expressed in human tumor-infiltrating lymphocytes in at least three different types of cancer including: glioma, head-and-neck squamous cell carcinoma and hepatocellular carcinoma. AF196970.3 levels of expression correlated with SUV39H1 levels and were higher in proliferating T cells, like SUV39H1.

The present disclosure also relates to the identification of short hairpin RNAs comprising any one of the target sequences as described in any one of SEQ ID NO: 13-17 (shRNA1-5 as herein described) or comprising any one of the sequences SEQ ID NO: 26-30 (comprising the target sequence, loop sequence and guide sequence to form the shRNA).

The disclosure provides inhibitory polynucleotides and uses thereof. SUV39H1 expression in cells can be inhibited by increasing ectopic or endogenous expression of this antisense lncRNA sequence, or shRNAs or similar polynucleotides based on this sequence, variants and/or fragments thereof, as disclosed herein. Decreased SUV39H1 levels in cells affects the cells' commitment to terminal differentiation and/or prolongs survival. This lncRNA sequence, or fragments or variants thereof including shorter RNAs or similar polynucleotides based on the lncRNA sequence, as well as shRNA as herein described typically including one of the target sequences SEQ ID NO: 13-17, or comprising any one of SEQ ID NO:26-30could either function as a local inhibitor of SUV39H1 transcription and/or function via its overlapping anti-sense exons. In any of these embodiments, inhibition of SUV39H1 reduces SUV39H1 expression and/or activity by at least about 20%, 30%, 40%, 50%, 60%, 70%, 75%, or 80% or more compared to wild type cells in which SUV39H1 expression has not been modulated. In some embodiments, RNA inhibitors as herein described reduced H3K9 tri-methylation by at least about 20%, 30%, 40%, 50%, 60%, 70%, 75%, or 80% or more compared to wild type cells in which SUV39H1 expression has not been modulated (see also the result section for an example assay).

The disclosure also provides activating polynucleotides and uses thereof. SUV39H1 expression in cells is increased by inhibiting expression or increasing degradation of the lncRNA sequence, AF196970.3. RNA interference (RNAi), short hairpin RNAs (shRNA), and antisense oligonucleotides (ASO) can be used to inhibit expression or decrease degradation of RNAs.

Immune cells, particularly T-cells or NK cells, in which expression of SUV39H1 has been inhibited may exhibit an enhanced central memory phenotype, enhanced survival and persistence after adoptive transfer, and reduced exhaustion. In particular, such cells accumulate and re-program with increased efficiency into long-lived central memory T cells. Such cells are more efficient at inducing tumor cell rejection and display enhanced efficacy for treating cancer.

### Definitions

As used herein, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, ±5%, ±1%, ±0.5%, or even ±0.1% of the specified amount.

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, variable heavy chain (VH) regions capable of specifically binding the antigen, single chain antibody fragments, including single chain variable fragments (scFv), and single domain antibodies (e.g., sdAb, sdFv, nanobody) fragments. The term encompasses recombinant and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgG1, IgG2, IgG3, IgG4, IgM, IgE, IgA, and IgD. In some embodiments the antibody comprises a heavy chain variable region and a light chain variable region.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; variable heavy chain (VH) regions, VHH antibodies, single-chain antibody molecules such as scFvs and single-domain VH single antibodies; and multispecific antibodies formed from antibody fragments. In particular embodiments, the antibodies are single-chain antibody fragments comprising a variable heavy chain region and/or a variable light chain region, such as scFvs.

“Inactivation” or “disruption” of a gene refers to a change in the sequence of genomic DNA that causes the gene's expression to be reduced or eliminated, or that cause a non-functional gene product to be expressed. Exemplary methods include gene silencing, knockdown, knockout, and/or gene disruption techniques, such as gene editing through, e.g., induction of breaks and/or homologous recombination. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination.

As used herein, “inhibition” of a gene product refers to a decrease of its activity and/or gene expression of at least 20%, 30%, 40%, 50%, 60%, 70%, 80%, 90%, 95% or 99% or more as compared to the activity or expression levels of wildtype which is not inhibited or repressed.

As used herein, “express” or “expression” means that a gene sequence is transcribed, and optionally, translated. If the gene expresses a noncoding RNA, expression will typically result in an RNA after transcription and, optionally, splicing. If the gene is a coding sequence, expression will typically result in production of a polypeptide after transcription and translation.

As used herein, “expression control sequence” refers to a nucleotide sequence that influences the transcription, RNA processing, RNA stability, or translation of the associated nucleotide sequence. Examples include, but are not limited to, promoters, enhancers, introns, translation leader sequences, polyadenylation signal sequences, transcription initiators and transcriptional and/or translational termination region (i.e., termination region).

As used herein, “fragment” means a portion of a referenced sequence (polynucleotide or polypeptide) that has a length at least 60%, 65%, 70%, 75%, 80%, 85%, 90%, 95%, 96%, 97%, 98%, or 99% of the full-length sequence.

As used herein, “heterologous” refers to a polynucleotide or polypeptide that comprises sequences that are not found in the same relationship to each other in nature. For example, the heterologous sequence either originates from another species, or is from the same species or organism but is modified from either its original form or the form primarily expressed in the cell. Thus, a heterologous polynucleotide includes a nucleotide sequence derived from and inserted into the same natural, original cell type, but which is present in a non-natural state, e.g., a different copy number, and/or under the control of different regulatory sequences than that found in nature and/or located in a different position (adjacent to a different nucleotide sequence) than where it was originally located.

As used herein, “nucleic acid,” “nucleotide sequence,” and “oligonucleotide” or “polynucleotide” are used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present disclosure further provides a nucleic acid that is the complement (which can be either a full complement or a partial complement) of a nucleic acid, nucleotide sequence, or polynucleotide described herein. Modified bases (modified nucleobases), such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, “operably linked” means that an element, such as an expression control sequence, is configured so as to perform its usual function upon a nucleotide sequence of interest. For example, a promoter operably linked to a nucleotide sequence of interest is capable of effecting expression of the nucleotide sequence of interest. The expression control sequences need not be contiguous with the nucleotide sequence of interest, so long as they function to direct the expression thereof.

As used herein, the expression “percentage of identity” between two sequences, means the percentage of identical bases or amino acids between the two sequences to be compared, obtained with the best alignment of said sequences, this percentage being purely statistical and the differences between these two sequences being randomly spread over the two sequences. A base is considered complementary if it hybridizes under normal conditions. For example, a modified nucleobase can be aligned in a manner like the base whose hybridization pattern it mimics. As used herein, “best alignment” or “optimal alignment”, means the alignment for which the determined percentage of identity (see above) is the highest. Sequence comparison between two nucleic acid sequences (also referenced herein as nucleotide sequence or nucleobase sequence) is usually realized by comparing these sequences that have been previously aligned according to the best alignment. This comparison is realized on segments of comparison in order to identify and compared the local regions of similarity. The best sequence alignment to perform comparison can be realized, besides manually, by using the global homology algorithm developed by SMITH and WATERMAN (Ad. App. Math., vol. 2, p: 482, 1981), by using the local homology algorithm developed by NEEDLEMAN and WUNSCH (J. Mol. Biol, vol. 48, p: 443, 1970), by using the method of similarities developed by PEARSON and LIPMAN (Proc. Natl. Acad. Sci. USA, vol. 85, p: 2444, 1988), by using computer software using such algorithms (GAP, BESTFIT, BLAST P, BLAST N, FASTA, TFASTA in the Wisconsin Genetics software Package, Genetics Computer Group, 575 Science Dr., Madison, WI, USA), by using the MUSCLE multiple alignment algorithms (Edgar, Robert C, Nucleic Acids Research, vol. 32, p: 1792, 2004). To get the best local alignment, one can preferably use BLAST software. The identity percentage between two sequences is determined by comparing these two sequences optimally aligned, the sequences being able to comprise additions or deletions with respect to the reference sequence in order to get the optimal alignment between these two sequences. The percentage of identity is calculated by determining the number of identical positions between these two sequences and dividing this number by the total number of compared positions, and by multiplying the result obtained by 100 to get the percentage of identity between these two sequences.

As used herein, “treatment”, or “treating” involves application of cells of the disclosure or a composition comprising the cells to a patient in need thereof with the purpose to cure, heal, alleviate, relieve, alter, remedy, ameliorate, improve or affect the disease such as cancer, or any symptom of the disease (e.g., cancer). In particular, the terms “treat” or treatment” refers to reducing or alleviating at least one adverse clinical symptom associated with the disease. With reference to cancer treatment, the term “treat” or “treatment” also refers to slowing or reversing the progression of neoplastic uncontrolled cell multiplication, i.e. shrinking existing tumors and/or halting tumor growth. The term “treat” or “treatment” also refers to inducing apoptosis in cancer or tumor cells in the subject.

As used herein, “variant” means a sequence (polynucleotide or polypeptide) that has mutations (deletion, substitution or insertion) that is at least 60%, 65%, 70%, 75%, 80%, 85%, 90%, 95%, 96%, 97%, 98%, or 99% identical to a referenced sequence over its full length or over a region of at least about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, or 1100 nucleotides or amino acids. With respect to a polynucleotide sequence, variant also encompasses a polynucleotide that hybridizes under stringent conditions to the referenced sequence or complement thereof.

As used herein, a “vector” is any nucleic acid molecule for the transfer into or expression of a nucleic acid in a cell. The term “vector” includes both viral and nonviral (e.g., plasmid) nucleic acid molecules for introducing a nucleic acid into a cell in vitro, ex vivo, and/or in vivo. Vectors may include expression control sequences, restriction sites, and/or selectable markers. A “recombinant” vector refers to a vector that comprises one or more heterologous nucleotide sequences.

Inhibitory Polynucleotides that Inhibit SUV39H1 Expression

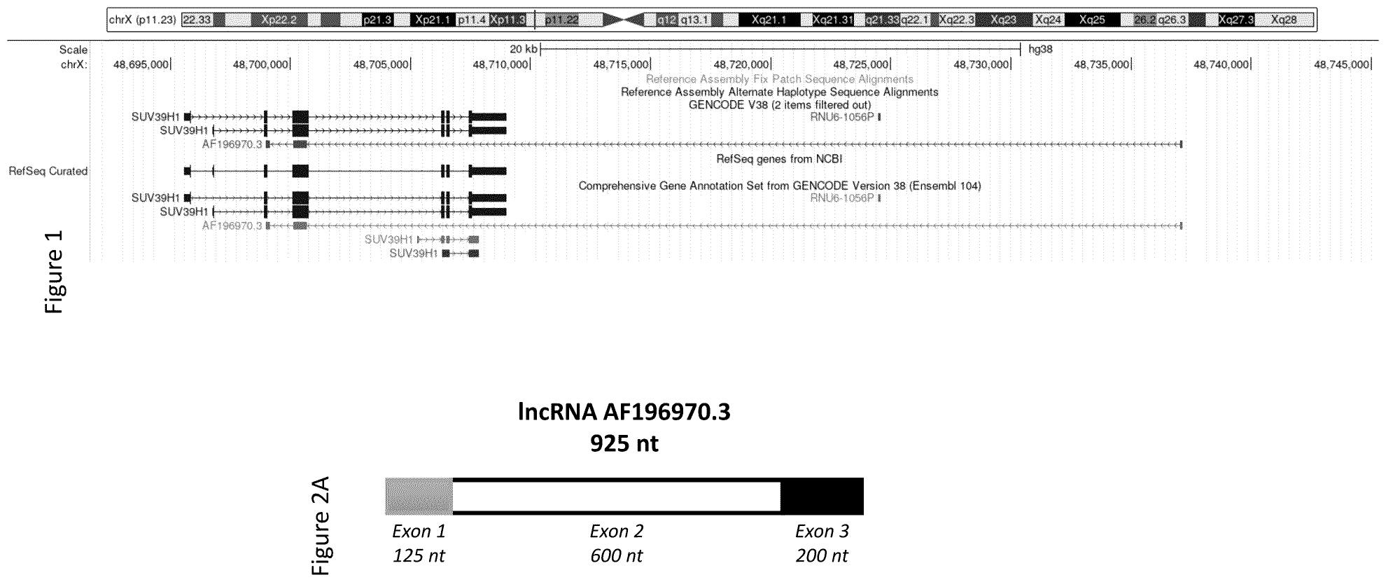

The gene locus of SUV39H1 is located on the X chromosome (position p11.23, 48695554-48709016, in the GRCh38.p13 assembly). At the same locus, in anti-parallel orientation, the non-annotated gene ENSG00000232828 is located at positions 48698963-48737163 (FIG. 1). Both genes have annotated promoter regions.

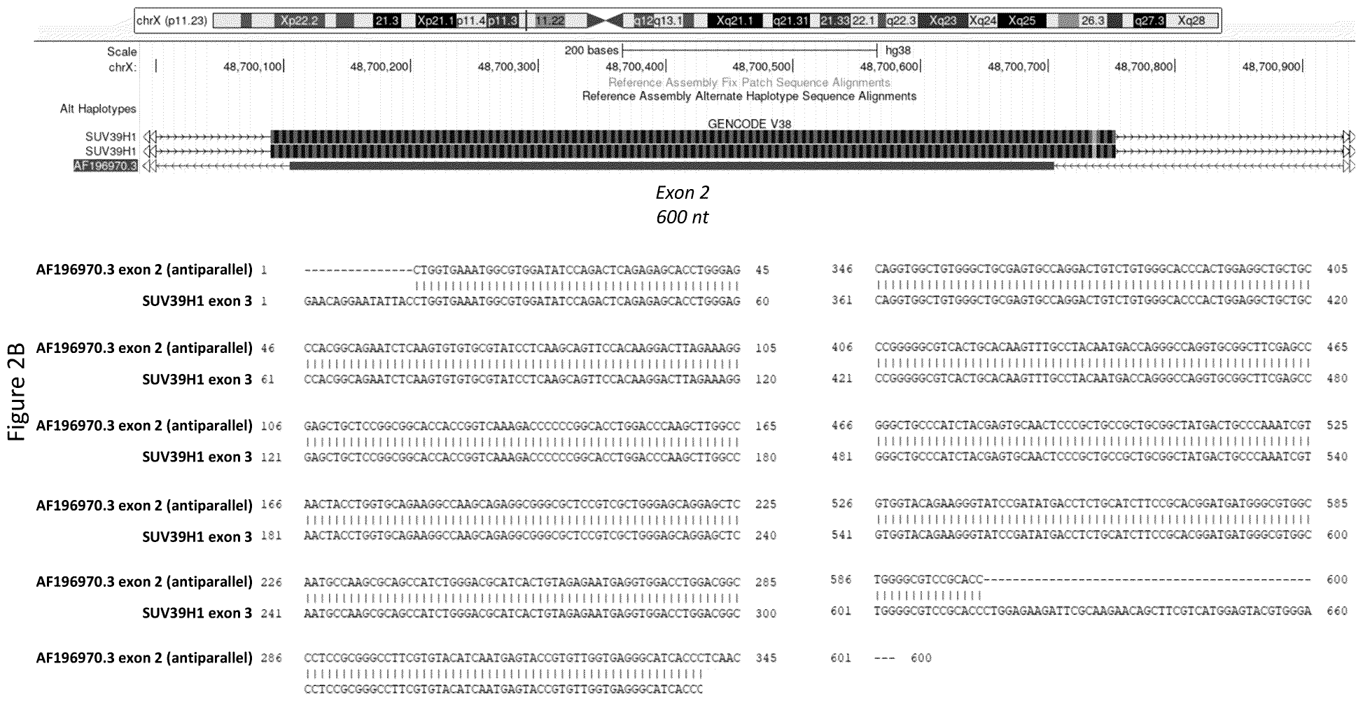

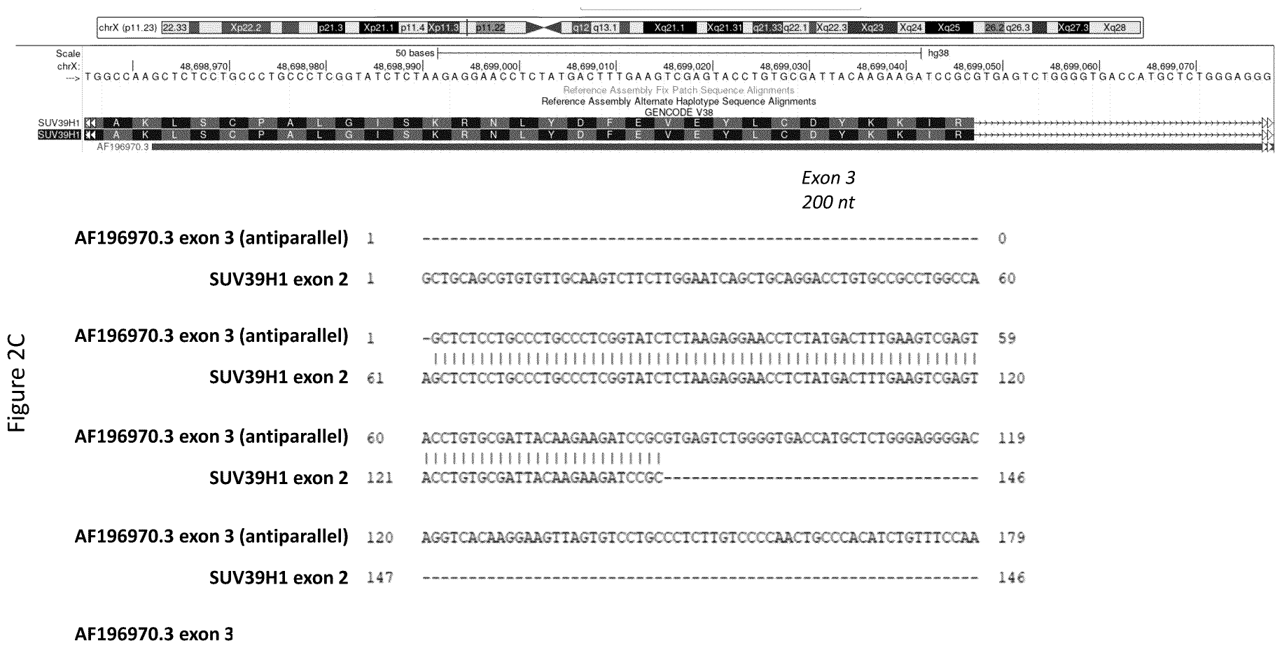

AF196970.3 (SEQ ID NO: 1) is the predicted RNA sequence expressed from ENSG00000232828 (SEQ ID NO: 5), after transcription and splicing. SEQ ID NO: 1 is a 925 base sequence that includes three exons (FIG. 2A). AF196970.3 exon 1 (SEQ ID NO: 2) is 125 bases in length and does not have significant complementarity to the SUV39H1 gene. AF196970.3 exon 2 (SEQ ID NO: 3) is 600 bases in length and is anti-parallel to a substantial portion of exon 3 of the SUV39H1 gene with 100% similarity (FIG. 2B). AF196970.3 exon 3 (SEQ ID NO: 4) is 200 bases in length and is anti-parallel to a portion (42.5% similarity) of exon 2 of the SUV39H1 gene, as well as a portion of the adjacent intron (FIG. 2C).

Inhibitory polynucleotides of the disclosure, for use in the cells and methods of the disclosure, include AF196970.3 (SEQ ID NO: 1) or fragments or variants thereof. Such fragments include fragments of any of SEQ ID NOs: 1-4, of about 12-50, 50-100, 100-150, 150-200, 200-250, 250-300, 300-350, 350-400, 400-450, 450-500, 500-550, 550-600, 600-650, 650-700, 700-750, 750-800, 800-850, 850-900, 250-750, 500-750 or more bases in length. Variants include polynucleotides, or chemically modified polynucleotides, in which SEQ ID NO: 1 or the foregoing fragments of SEQ ID NO: 1 have been mutated, including deletions, substitutions, modifications (including chemical modifications), or insertions. Such variants can have a nucleobase sequence at least about 70%, 75%, 80%, 85%, 90%, 95%, 97%, 98% or 99% identical to any of SEQ ID NOs: 1-4 or any fragments thereof, for example, fragments of SEQ ID NO: 1 of the aforementioned lengths. Variants include, for example, any of the foregoing fragments of SEQ ID NO: 1 linked to additional nucleotides at either the 5′ end or 3′ end or both. Variants also include RNA produced by alternative splicing of ENSG00000232828 (SEQ ID NO: 5) or RNA produced by allelic variants of ENSG00000232828 (SEQ ID NO: 5).

Inhibitory RNAs as per the present disclosure also include shRNA as herein described. Principles and design of shRNA is for example described in Moore C B, Guthrie E H, Huang M T, Taxman D J. Short hairpin RNA (shRNA): design, delivery, and assessment of gene knockdown. Methods Mol Biol. 2010; 629:141-58, but see also Taxman D J. siRNA and shRNA design. In: Helliwell TDC, editor. RNA Interference Methods for Plants and Animals. Vol. 10. CABI; Oxfordshire, UK: 2009. pp. 228-253. The mechanism of RNAi is based on the sequence-specific degradation of host mRNA through the cytoplasmic delivery of double-stranded RNA (dsRNA) identical to the target sequence. shRNAs may be transfected as plasmid vectors encoding shRNAs transcribed by heterologous promoters, but can also be delivered into mammalian cells through infection of the cell with virally produced vectors. shRNAs are capable of DNA integration and typically consist of two complementary 19-22 bp RNA sequences linked by a short loop of 4-11 nt similar to the hairpin found in naturally occurring miRNA. Examples loop sequences are also described herein but any variation thereof based on the classical knowledge of the skilled person can be used (see also the above references for the design of shRNAs). Typically, an shRNA comprises or is encoded by a nucleotide sequence comprising one of the sequences of SEQ ID NO: 13-17 and 26-30 and SEQ ID NO:32-36 and 45-49 respectively.

Chemical modifications include one or more of a modified backbone linkage, a modified sugar moiety, a modified phosphate moiety, a modified nucleobase, or a chemically conjugated moiety. Such chemical modifications can be present throughout the polynucleotide, or in alternating patterns. Chemical modifications can be present at the 5′ end or 3′ end or both, e.g. the 5-10 bases at the 5′ and/or 3′ end comprise one or more of a modified backbone linkage, a modified sugar moiety, a modified phosphate moiety, a modified nucleobase, or a chemically conjugated moiety.

Such inhibitory polynucleotides can be delivered directly, as heterologous RNA or as heterologous polynucleotides, e.g., chemically modified polynucleotides that have been modified to increase their serum half-life and/or affinity. Alternatively, such inhibitory polynucleotides can be expressed ectopically from DNA or vectors expressing such inhibitory polynucleotides. In yet another alternative, endogenous expression of such inhibitory polynucleotides can be upregulated, e.g., by inserting expression control sequences, such as constitutive, inducible, strong or tissue-specific promoters.

Activating Polynucleotides that Increase SUV39H1 Expression

Activating polynucleotides include agents known in the art for inhibiting expression of or increasing degradation of this lncRNA sequence, including RNA interference (RNAi), short hairpin RNAs (shRNA), antisense oligonucleotides (ASO), or ribozymes, each of which include a segment complementary to the lncRNA sequence. Also contemplated are ZF or ZFN, TALE or TALEN, or CRISPR systems such as CRISPR-Cas9 or CRISPR-Cas13 which comprise a guide RNA, each comprising a segment complementary to the lncRNA or the gene encoding or expressing the lncRNA. The region of complementarity may be at least about 12-20, 12-18 or 15-18 bases in length.

Typically, after transfection of siRNAs or expression of shRNAs, RNAi triggers the degradation of target RNA molecules through direct complementarity, mediated by the RNA-induced silencing complex. An alternative to RNAi for the degradation of lncRNAs are ASOs (FIG. 2B). ASOs are 15-20-nucleotide single-stranded DNA oligomers that are typically chemically modified to increase the efficacy of knockdown and decrease in vivo toxicity. In particular, the 2′-MOE and LNA gapmer modifications have been shown to increase affinity toward target RNA transcripts and endow resistance to nucleases, allowing these modified ASOs to have half-lives between days to several weeks in vivo. ASOs hybridize with target RNA transcripts through complementarity and induce RNaseH-mediated degradation of the target transcripts.

CRISPR-Cas13 can also efficiently cleave the RNA targets when provided with sgRNAs complementary to the target RNA. Cas13 has been used to knockdown lncRNAs in mammalian cells. CRISPRi or Zinc finger (ZF) or a TALE protein, where dCas9, TALE and/or ZF are linked directly or indirectly to a repressor and/or inhibitor, are also capable of repressing expression of RNAs. Finally, expression of lncRNA can be eliminated by deleting or replacing all or a portion of the encoding gene, e.g. through CRISPR-Cas9, ZF nuclease (ZFN) or TALE nuclease (TALEN) gene editing. See Liu and Lim, EMBO Reports (2018) 19: e46955.

### Expression of Inhibitory Polynucleotides or Activating Polynucleotides

The disclosure also provides a nucleic acid which encodes or expresses (e.g., is a template for production of) AF196970.3 (SEQ ID NO: 1), or any one of SEQ ID NO: 13-17 and 26-30 or a fragment or variant thereof capable of inhibiting expression of SUV39H1 in a cell. In some embodiments, the nucleic acid is heterologous to the cell and is operably linked to an expression control sequence. In other embodiments, the nucleic acid is native to the cell but operatively linked to a heterologous expression control sequence. Examples of such nucleic acids include ENSG00000232828 (SEQ ID NO: 5) or the cDNA of SEQ ID NO: 6, or of SEQ ID NO: 32-36 and 45-49 or a fragment or variant thereof. Other examples include nucleic acids that encodes any of exons 1-3 (SEQ ID NO: 2-4), or a fragment or variant thereof. Yet other examples of such nucleic acids include any of the cDNAs of SEQ ID NO: 6-9, 32-36 and 45-49 or a fragment or variant thereof. Such nucleic acids may be part of plasmids or vectors or transposase systems.

In some embodiments, expression of an endogenous nucleic acid that encodes or expresses AF196970.3 (SEQ ID NO: 1) or any one of the shRNAs as herein described (SEQ ID NO: 13-17 or 26-30) or a fragment or variant thereof capable of inhibiting expression of SUV39H1, is upregulated in the cell, for example, by operatively linking the endogenous nucleic acid to a heterologous expression control sequence (e.g., promoter, enhancer, other regulatory sequence). In some embodiments, expression of the endogenous gene or allelic variant thereof, e.g., ENSG00000232828 (SEQ ID NO: 5), is upregulated in the cell, for example, by operatively linking the endogenous nucleic acid to a heterologous expression control sequence.

Means and vectors for expressing polynucleotides such as RNA are well known in the art and commercially available. Known vectors include viral vectors and pseudotyped viral vectors, such as retroviral, lentivirus, adenoviral, adeno-associated (AAV), alphavirus, vaccinia virus, poxvirus, EBV, and herpes simplex virus papillomavirus, foamivirus, or Semliki Forest virus vectors, or transposase systems, such as Sleeping Beauty transposase vectors. Non-viral systems for delivery of naked plasmids to cells include lipids, cationic lipid complexes, liposomes, nanoparticle, gold particle, or polymer complex, poly-lysine conjugates, synthetic polyamino polymers and artificial viral envelopes.

For expressing short noncoding RNAs, such as shRNA, polymerase III promoters are commonly used. Examples include U6, H1, or 7SK promoters.

For expressing long RNAs, such as mRNAs or long noncoding RNAs, polymerase II promoters are commonly used. Examples include CMV, EF-1a, hPGK and RPBSA. CAG promoters have been used to overexpress lncRNA. Yin et al Cell Stem Cell. 2015 May 7; 16(5): 504-16. Inducible promoters that are driven by signals from activated T cells include nuclear factor of activated T cells (NFAT) promoter. Other promoters for T cell expression of RNA include CIFT chimeric promoter (containing portions of cytomegalovirus (CMV) enhancer, core interferon gamma (IFN-γ) promoter, and a T-lymphotropic virus long terminal repeat sequence (TLTR)), endogenous TRAC promoter or TRBC promoter. Inducible, constitutive, or tissue-specific promoters are contemplated.

Viral vectors have been used to overexpress lncRNAs, see Yang et al. (2013) Mol Cell 49:1083-1096; Lu et al. (2018) Mol Ther Nucleic Acids 10:387-397. In addition, lncRNA has been expressed from lentivectors and SB transposase systems. See, e.g., Zhang et al., Overexpression of lncRNAs with endogenous lengths and functions using a lncRNA delivery system based on transposon. J Nanobiotechnol 19, 303 (2021).

CRISPR-Cas9-based activation systems have also been used to upregulate endogenous expression of lncRNA. Rankin et al., Overexpressing Long Noncoding RNAs Using Gene-activating CRISPR. J Vis Exp. 2019; (145): 10.3791/59233.

In some embodiments, dsRNA such as RNAi is produced in the cell by a vector comprising an expression control sequence operatively linked to a nucleotide sequence that expresses (is a template for one or both strands of) the dsRNA. In further embodiments, a promoter can flank either end of the template nucleotide sequence, wherein the promoters drive expression of each individual DNA strand, thereby generating two complementary (or substantially complementary) RNAs that hybridize and form the dsRNA. In other embodiments, dsRNA is produced in the cell by a vector that expresses an shRNA that is processed to form an interfering dsRNA.

### Delivery of Nucleic Acids or Polynucleotides

Art-recognized techniques for introducing foreign nucleic acids (e.g., DNA and RNA) into a host cell, include calcium phosphate or calcium chloride co-precipitation, DEAE-dextran-mediated transfection, lipofection, electroporation, microinjection, DNA-loaded liposomes, lipofectamine-DNA complexes, cell sonication, gene bombardment using high velocity microprojectiles, biolistics, and viral-mediated transfection. Compositions comprising the nucleic acids may also comprise transfection facilitating agents, which include surface active agents, quinone analogs, vesicles such as squalene and squalene, hyaluronic acid, lipids, liposomes, lecithin liposomes, calcium ions, viral proteins, polyanions, polycations, including poly-L-glutamate, or nanoparticles, gold particles, or other known agents. Delivery vehicles include a liposome, lipid-containing complex, nanoparticle, gold particle, or polymer complex.

Lipid materials have been used to create lipid nanoparticles (LNPs) based on ionizable cationic lipids, which exhibit a cationic charge in the lowered pH of late endosomes to induce endosomal escape, because of the tertiary amines in their structure. These LNPs have been used, for example, to deliver RNA interference (RNAi) components, as well as genetic constructs or CRISPR-Cas systems. See, such as, Wilbie et al., Acc Chem Res.; 52(6): 1555-1564, 2019. Wang et al., Proc Natl Acad Sci USA.; 113(11): 2868-2873, 2016 describe use of biodegradable cationic LNPs. Chang et al., Acc. Chem. Res., 52, 665-675, 2019 describe use of ionizable lipid along with cholesterol, DSPC, and a PEGylated lipid to create LNPs.

Polymer based particles can be used for genetic construct delivery in a similar manner as lipids. Numerous materials have been used for delivery of nucleic acids. For example, cationic polymers such as polyethylenimine (PEI) can be complexed to nucleic acids and can induce endosomal uptake and release, similarly to cationic lipids. Dendrimeric structures of poly(amido-amine) (PAMAM) can also be used for transfection. These particles consist of a core from which the polymer branches. They exhibit cationic primary amines on their surface, which can complex to nucleic acids. Networks based on zinc to aid cross-linking of imidazole have been used as delivery methods, relying on the low pH of late endosomes which, upon uptake, results in cationic charges due to dissolution of the zeolitic imidazole frameworks (ZIF), after which the components are released into the cytosol. Colloidal gold nanoparticles have also been used. See Wilbie et al., supra.

### Production and Chemical Modification of Inhibitory Polynucleotides or Activating Polynucleotides

In vitro transcribed, chemically synthesized, or partially chemically synthesized RNAs can be delivered. For example, an RNA may be in vitro transcribed and chemically linked to chemically synthesized RNA at the 5′ end and/or 3′ end. Direct injection or transfection of in vitro-transcribed lncRNAs has been performed to demonstrate lncRNA function. Ulitsky et al. (2011) Cell, 147(7): 1537-1550.

Chemical modifications to oligonucleotides (also referred to as polynucleotides) can improve their resistance to degradation, thereby increasing half-life, and/or increase affinity for complementary polynucleotides. Modified oligonucleotides (e.g., RNA oligonucleotides) comprise chemical modifications including one or more of a modified backbone linkage, a modified phosphate moiety, a modified sugar moiety, a modified nucleobase, or a chemically conjugated moiety, or any combinations thereof. One, two, three, four, five, 10, 15, 20 or more of the same type of modification, optionally in combination with 1, 2, 3, 4, 5, 10, 15, 20 or more of another type of modification, or patterns of modifications are contemplated. Patterns include alternating modifications throughout the oligonucleotide, such as 2′-fluoro and 2′-methoxy, or end modifications wherein, for example, 1, 2, 3, 4, 5, or more of the bases, sugars, or linkages at the 5′ and/or 3′ end of the oligonucleotide are modified.

Examples of modified backbone linkages include phosphorothioate, phosphothioate (PhTx) group or phosphonoacetate, thiophosphonoacetate, methylphosphonate, boranophosphate, or phosphorodithioate. Other internucleotide bridging modified phosphates may be used, such as methyl phosphonothioates, phosphoromorpholidates, phosphoropiperazidates and phosphoramidates. For example, every one or every other one of the internucleotide bridging phosphate residues can be modified as described.

Examples of modified sugar moieties include deoxyribose, or replacement of the 2′ OH-group by another group. Although the majority of sugar analog alterations are localized to the 2′ position, other sites are amenable to modification, including the 4′ position. Example replacement groups include H, —OR, —R (wherein R can be, such as, alkyl, cycloalkyl, aryl, aralkyl, heteroaryl or sugar), lower alkyl moiety (e.g., C1-C4, linear or branched, saturated or unsaturated alkyl, such as methyl, ethyl, ethenyl, propyl, 1-propenyl, 2-propenyl, and isopropyl), halo, —F, —Br, —Cl or —I, —SH, —SR (wherein R can be, such as, alkyl, cycloalkyl, aryl, aralkyl, heteroaryl or sugar), -arabino, F-arabino, amino (wherein amino can be, such as, NH₂; alkylamino, dialkylamino, heterocyclyl, arylamino, diarylamino, heteroarylamino, diheteroarylamino, or amino acid); or cyano (—CN). Specific examples include a 2′-fluoro sugar, 2′-O-methyl sugar, 2′-O-methoxyethyl sugar, or a locked nucleic acid (LNA) nucleotide. Specific examples of modified 2′-sugar include 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof. For example, every one or every other one of the nucleotides can be modified as described.

Examples of modified nucleobases include, but are not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomet-hyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

Polynucleotides can also be stabilized by complexing to lipids or liposomes. In some embodiments, the liposome comprises 1,2-diolcoyl-sn-glycero-3-phosphatidylcholine (DOPC). In certain embodiments, the lipid particle comprises cholesterol, 1,2-distearoyl-sn-glycero-3-phosphocholine (DSPC), PEG-CDMA or PEG-cDSA, and 1,2-dilinoleyloxy-3-(N,N-dimethyl) aminopropane (DLinDMA).

Cells with Modulated SUV39H1 Expression

Cells according to the disclosure exhibit modulation, preferably inhibition, of SUV39H1 expression. The cells are typically mammalian cells, or cell lines, e.g., mouse, rat, pig, non-human primate, or preferably human. Such cells include cells derived from the blood, bone marrow, lymph, or lymphoid organs (notably the thymus) and are preferably cells of the immune system (i.e., immune cells), such as cells of the innate or adaptive immunity, e.g., myeloid or lymphoid cells, including monocytes, macrophages, dendritic cells, or lymphocytes, typically T cells and/or NK cells. Immune cells or progenitors thereof preferably also comprise one or more, or two or more, or three or more antigen-specific receptors (CAR and/or TCR) as described herein, and optionally comprise one or more co-stimulatory receptors. Among the antigen-specific receptors according to the disclosure are recombinant modified T cell receptors (TCRs) and components thereof, as well as functional non-TCR antigen-specific receptors, such as chimeric antigen receptors (CAR).

Cells according to the disclosure may also be immune cell progenitors, such as lymphoid progenitors and more preferably T cell progenitors. Examples of T-cell progenitors include pluripotent stem cells (PSC), induced pluripotent stem cells (iPSCs), hematopoietic stem cells (HSC), human embryonic stem cells (ESC), adipose-derived stem cells (ADSC), multipotent progenitor (MPP); lymphoid-primed multipotent progenitor (LMPP); common lymphoid progenitor (CLP); lymphoid progenitor (LP); thymus settling progenitor (TSP); or early thymic progenitor (ETP). Hematopoictic stem and progenitor cells can be obtained, for example, from cord blood, or from peripheral blood, e.g. peripheral blood-derived CD34+ cells after mobilization treatment with granulocyte-colony stimulating factor (G-CSF). T cell progenitors typically express a set of consensus markers including CD44, CD117, CD135, and/or Sca-1.

In some embodiments, the cells include one or more subsets of T cells or other cell types, such as whole T cell populations, CD4+ and/or CD8+ T cells, and subpopulations thereof, such as those defined by function, activation state, maturity, potential for differentiation, expansion, recirculation, localization, and/or persistence capacities, antigen-specificity, type of antigen-specific receptor, presence in a particular organ or compartment, marker or cytokine secretion profile, and/or degree of differentiation. In some embodiments, the cells include myeloid derived cells, such as dendritic cells, monocytes or macrophages.

Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. Specifically contemplated herein are TEFF cells with stem/memory properties and higher reconstitution capacity due to the inhibition of SUV39H1, as well as TN cells, TSCM, TCM, TEM cells and combinations thereof.

In some embodiments, one or more of the T cell populations is enriched for, or depleted of, cells that are positive for or express high levels of one or more particular markers, such as surface markers, or that are negative for or express relatively low levels of one or more markers. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD117, CD135, CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, and/or IL7-Ra (CD127). In some examples, CD8+ T cells are enriched for cells positive for CD45RO (or negative for CD45RA) and for CD62L. The subset of cells that are CCR7+, CD45RO+, CD27+, CD62L+ cells constitute a central memory cell subset.

For example, according to the disclosure, the cells can include a CD4+ T cell population and/or a CD8+ T cell sub-population, e.g., a sub-population enriched for central memory (TCM) cells. Alternatively, the cells can be other types of lymphocytes, including natural killer (NK) cells, mucosal associated invariant T (MAIT) cells, Innate Lymphoid Cells (ILCs) and B cells.

Cells include primary cells, isolated directly from a biological sample obtained from a subject, and optionally frozen. In some embodiments, the subject is in need of a cell therapy (adoptive cell therapy) and/or is the one who will receive the cell therapy. With reference to a subject to be treated with cell therapy, the cells may be allogeneic and/or autologous. In autologous immune cell therapy, immune cells are collected from the patient, modified as described herein, and returned to the patient. In allogeneic immune cell therapy, immune cells are collected from healthy donors, rather than the patient, modified as described herein, and administered to patients. Typically, these are HLA matched to reduce the likelihood of rejection by the host. The immune cells may also comprise modifications to reduce immunogenicity such as disruption or removal of HLA class I molecules, HLA-A locus, and/or Beta-2 microglobulin (B2M).

Universal “off the shelf product” immune cells typically comprise modifications designed to reduce graft vs. host disease, such disruption or deletion of endogenous TCR. Because a single gene encodes the alpha chain (TRAC) rather than the two genes encoding the beta chain (TRBC), the TRAC locus is a common target for removing or disrupting endogenous TCR expression.

The samples include tissue samples, from tissues or organ, or fluid samples, such as blood, plasma, serum, cerebrospinal fluid, or synovial fluid. Samples may be taken directly from the subject, or may result from one or more processing steps, such as separation, centrifugation, genetic engineering (for example transduction with viral vector), washing, and/or incubation. Blood or blood-derived samples may be derived from an apheresis or a leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, myeloid derived cells, and/or cells derived therefrom.

### Methods for Producing Cells

Provided herein are methods of producing the cells of the disclosure with modulated SUV39H1 expression. For cells with inhibited SUV39H1 expression, such methods include introducing into such a cell one or more, or two or more, or three or more, of the inhibitory polynucleotides disclosed herein. More specifically, such methods include introducing into the cell a polynucleotide comprising the nucleobase sequence of AF196970.3 (SEQ ID NO: 1) or fragment or variant thereof capable of inhibiting expression of SUV39H1 in the cell, as described herein, in an amount and under conditions effective to increase SUV39H1 expression. Other examples include polynucleotides comprising the nucleobase sequence of any of exons 1-3 (SEQ ID NO: 2-4), or a fragment or variant thereof capable of inhibiting expression of SUV39H1 in the cell. Such polynucleotides may be chemically modified to reduce degradation and/or increase affinity.

Such methods also include introducing into the cell a nucleic acid, plasmid, vector or transposase system encoding or expressing AF196970.3 (SEQ ID NO: 1) or an shRNA as herein described (comprising one of the sequence SEQ ID NO: 13-17 or 26-30 as herein described) or a fragment or variant thereof capable of inhibiting expression of SUV39H1 in the cell, preferably operably linked to an expression control sequence, in an amount and under conditions effective to increase SUV39H1 expression. Examples of such nucleic acids include ENSG00000232828 (SEQ ID NO: 5) or the cDNA of SEQ ID NO: 6, 32-36 and 45-49 or a fragment or variant thereof. Other examples include nucleic acids that encodes any of exons 1-3 (SEQ ID NO: 2-4), or a fragment or variant thereof. Yet other examples of such nucleic acids include any of the cDNAs of SEQ ID NO: 6-9 or a fragment or variant thereof.

Such methods also include upregulating endogenous expression of a nucleic acid encoding or expressing AF196970.3 (SEQ ID NO: 1) or an shRNA as herein described (comprising any one of SEQ ID NO: 13-17 and 26-30 as herein described) or a fragment or variant thereof capable of inhibiting expression of SUV39H1 in the cell, by inserting into the genome of the cell a heterologous expression control sequence (e.g., promoter, enhancer, other regulatory sequence, constitutive, inducible or tissue-specific) operatively linked to said nucleic acid. In some embodiments, expression of the endogenous gene or allelic variant thereof, e.g., ENSG00000232828 (SEQ ID NO: 5), is upregulated in the cell.

A heterologous expression control sequence can be inserted into a cell, for example, by homologous recombination with a donor template after cleavage with known nuclease systems such as ZFNs, TALENs or CRISPR systems comprising a guide RNA (e.g., CRISPR-Cas9).

For cells with increased SUV39H1 expression, such methods include introducing into such a cell one or more, or two or more, or three or more, of the activating polynucleotides described herein, e.g. an RNAi, shRNA, ASO, ribozyme, or ZFN or TALEN or CRISPR system comprising a guide RNA, in an amount and under conditions effective to increase SUV39H1 expression.

### Antigen-Specific Receptors

The cells of the disclosure with modulated SUV39H1 expression include immune cells that express one or more, or two or more, or three or more antigen-specific receptors on their surface, and optionally one or more co-stimulatory receptors. Antigen-specific receptors include recombinant or modified T cell receptors (TCRs) and components thereof, and/or chimeric antigen receptors (CAR). For example, at least two CAR, at least two TCR, or at least one CAR with at least one TCR are contemplated. The antigen-specific receptors may bind the same or different antigen. In some embodiments, the two or more antigen-specific receptors have different signaling domains. In some embodiments, the cell comprises an antigen-specific receptor with an activating signaling domain and an antigen-specific receptor with an inhibitory signaling domain. Typically, such antigen-specific receptors bind the target antigen with a Kd binding affinity of about 10⁻⁶M or less, about 10⁻⁷M or less, about 10⁻⁸M or less, about 10⁻⁹M or less, about 10⁻¹⁰M or less, or about 10⁻¹¹M or less (lower numbers indicating greater binding affinity).

The cells thus may comprise one or more nucleic acids that encode one or more antigen-specific receptors, optionally operably linked to a heterologous regulatory control sequence. Typically, the nucleic acids are heterologous, (i.e., for example which are not ordinarily found in the cell being engineered and/or in the organism from which such cell is derived). In some embodiments, the nucleic acids are not naturally occurring, including chimeric combinations of nucleic acids encoding various domains from multiple different cell types. The nucleic acids and their regulatory control sequences are typically heterologous. For example, the nucleic acid encoding the antigen-specific receptor may be heterologous to the immune cell and operatively linked to an endogenous promoter of the T-cell receptor such that its expression is under control of the endogenous promoter. In some embodiments, the nucleic acid encoding a CAR is operatively linked to an endogenous TRAC promoter.

The immune cells, particularly if allogeneic, may be designed to reduce graft vs. host disease, such that the cells comprise inactivated (e.g. disrupted or deleted) endogenous TCR. Because a single gene encodes the alpha chain (TRAC) rather than the two genes encoding the beta chain, the TRAC locus is a typical target for reducing TCR receptor expression. Thus, the nucleic acid encoding the antigen-specific receptor (e.g. CAR or TCR) may be integrated into the TRAC locus at a location, preferably in the 5′ region of the first exon (SEQ ID NO: 3), that significantly reduces expression of a functional TCR alpha chain. See, e.g., Jantz et al., WO 2017/062451; Sadelain et al., WO 2017/180989; Torikai et al., Blood, 119(2): 5697-705 (2012); Eyquem et al., Nature. 2017 Mar. 2; 543(7643): 113-117. Expression of the endogenous TCR alpha may be reduced by at least 50%, 60%, 70%, 75%, 80%, 85%, 90%, 95%, 98%, or 99%. In such embodiments, expression of the nucleic acid encoding the antigen-specific receptor is optionally under control of the endogenous TCR-alpha promoter.

### Chimeric Antigen Receptors (CARs)

In some embodiments, the engineered antigen-specific receptors comprise chimeric antigen receptors (CARs), including activating or stimulatory CARs, costimulatory CARs (see WO2014/055668), and/or inhibitory CARs (iCARs, see Fedorov et al., Sci. Transl. Medicine, 5(215) (December 2013)).

Chimeric antigen receptors (CARs), (also known as Chimeric immunoreceptors, Chimeric T cell receptors, Artificial T cell receptors) are engineered antigen-specific receptors, which graft an arbitrary specificity onto an immune effector cell (T cell). Typically, these receptors are used to graft the specificity of a monoclonal antibody onto a T cell, with transfer of their coding sequence facilitated by retroviral vectors.

CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

The CAR may include (a) an extracellular antigen-binding domain, (b) a transmembrane domain, (c) optionally a co-stimulatory domain, and (d) an intracellular signaling domain.

In some embodiments, the CAR is constructed with a specificity for a particular antigen (or marker or ligand), such as an antigen expressed in a particular cell type to be targeted by adoptive cell therapy, such as a cancer marker. The CAR typically includes in its extracellular portion one or more antigen binding molecules, such as one or more antigen-binding fragment, domain, or portion of an antibody, typically one or more antibody variable domains. For example, the extracellular antigen-binding domain may comprise a light chain variable domain or fragment thereof and/or a heavy chain variable domain or fragment thereof, typically as an scFv. In some embodiments, the CAR comprises an antibody heavy chain variable domain or fragment thereof that specifically binds the antigen.

The moieties used to bind to antigen include three general categories, either single-chain antibody fragments (scFvs) derived from antibodies, Fab's selected from libraries, or natural ligands that engage their cognate receptor (for the first-generation CARs). Successful examples in each of these categories are notably reported in Sadelain M, Brentjens R, Riviere I. The basic principles of chimeric antigen receptor (CAR) design. Cancer discovery. 2013; 3(4): 388-398 (see notably table 1) and are included in the present disclosure.

Antibodies include chimeric, humanized or human antibodies, and can be further affinity matured and selected as described above. Chimeric or humanized scFvs derived from rodent immunoglobulins (e.g. mice, rat) are commonly used, as they are easily derived from well-characterized monoclonal antibodies. Humanized antibodies contain rodent-sequence derived CDR regions. Typically the rodent CDRs are engrafted into a human framework, and some of the human framework residues may be back-mutated to the original rodent framework residue to preserve affinity, and/or one or a few of the CDR residues may be mutated to increase affinity. Fully human antibodies have no murine sequence and are typically produced via phage display technologies of human antibody libraries, or immunization of transgenic mice whose native immunoglobin loci have been replaced with segments of human immunoglobulin loci. Variants of the antibodies can be produced that have one or more amino acid substitutions, insertions, or deletions in the native amino acid sequence, wherein the antibody retains or substantially retains its specific binding function. Conservative substitutions of amino acids are well known and described above. Further variants may also be produced that have improved affinity for the antigen.

In some embodiments, the modified TCR or CAR contains a fragment of an antibody or an antigen-binding fragment (e.g. scFv, or variable heavy (VH) region or variable light (VL) region or 1, 2, or 3 CDRs of such VH and/or VL) that specifically recognizes an intracellular antigen, such as a tumor-associated antigen, presented on the cell surface as a MHC-peptide complex. Generally, a CAR containing an antibody or antigen-binding fragment that exhibits TCR-like specificity directed against peptide-MHC complexes also may be referred to as a TCR-like CAR.

The transmembrane domain in some embodiments is derived either from a natural or from a synthetic source. The transmembrane domain may be derived from the same receptor as the intracellular signaling domain, or a different receptor. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD 134, CD137, CD154, ICOS or a GITR, or NKG2D, OX40, 2B4, DAP10, DAP12, or CD40. For T cells, CD8, CD28, CD3 epsilon may be preferred. For NK cells, NKG2D, DAP10, DAP12 may be preferred. In some embodiments, the transmembrane domain is derived from CD28, CD8 or CD3-zeta. In some instances, the transmembrane domain is selected or modified by amino acid substitution to avoid binding of such domains to the transmembrane domains of the same or different surface membrane proteins to minimize interactions with other members of the receptor complex.

In some embodiments, a short oligo- or polypeptide linker, for example, a linker of between 2 and 10 amino acids in length, is present and forms a linkage between the transmembrane domain and the cytoplasmic signaling domain of the CAR.

The CAR generally includes at least one intracellular signaling component or components. First generation CARs typically had the intracellular domain from the CD3-zeta-chain, which is the primary transmitter of signals from endogenous TCRs. Second generation CARs typically further comprise intracellular signaling domains from various costimulatory protein receptors to the cytoplasmic tail of the CAR to provide additional signals to the T cell. Co-stimulatory domains include domains derived from human CD28, 4-1BB (CD137), ICOS, CD27, OX 40 (CD134), DAP10, DAP12, 2B4, CD40, FCER1G or GITR (AITR). For T cells, CD28, CD27, 4-1BB (CD137), ICOS may be preferred. For NK cells, DAP10, DAP12, 2B4 may be preferred. Combinations of two co-stimulatory domains are contemplated, e.g. CD28 and 4-1BB, or CD28 and OX40. Third generation CARs combine multiple signaling domains, such as CD3zeta-CD28-4-1BB or CD3zeta-CD28-OX40, to augment potency.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

In some aspects, the CAR includes a primary cytoplasmic signaling sequence that regulates primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR zeta, FcR gamma, FcR beta, CD3 gamma, CD3 delta, CD3 epsilon, CDS, CD22, CD79a, CD79b, and CD66d. In some embodiments, cytoplasmic signaling molecule(s) in the CAR contain(s) a cytoplasmic signaling domain, portion thereof, or sequence derived from CD3 zeta. The CAR can also include a signaling domain and/or transmembrane portion of a costimulatory receptor, such as CD28, 4-1BB (CD137), ICOS, CD27, OX 40 (CD134), DAP10, DAP12, 2B4, CD40, FCER1G or GITR (AITR). In some aspects, the same CAR includes both the activating and costimulatory components; alternatively, the activating domain is provided by one CAR whereas the costimulatory component is provided by another CAR recognizing another antigen.

The intracellular signaling domain can be from an intracellular component of the TCR complex, such as a TCR CD3 chain that mediates T-cell activation and cytotoxicity, e.g., the CD3 zeta chain. Alternative intracellular signaling domains include FceRIγ. The intracellular signaling domain may comprise a modified CD3 zeta polypeptide lacking one or two of its three immunoreceptor tyrosine-based activation motifs (ITAMs), wherein the ITAMs are ITAM1, ITAM2 and ITAM3 (numbered from the N-terminus to the C-terminus). The intracellular signaling region of CD3-zeta is residues 22-164 of SEQ ID NO: 10. ITAM1 is located around amino acid residues 61-89, ITAM2 around amino acid residues 100-128, and ITAM3 around residues 131-159. Thus, the modified CD3 zeta polypeptide may have any one of ITAM1, ITAM2, or ITAM3 inactivated, e.g. disrupted or deleted. Alternatively, the modified CD3 zeta polypeptide may have any two ITAMs inactivated, e.g. ITAM2 and ITAM3, or ITAM1 and ITAM2. Preferably, ITAM3 is inactivated, e.g. deleted. More preferably, ITAM2 and ITAM3 are inactivated, e.g. deleted, leaving ITAM1. For example, one modified CD3 zeta polypeptide retains only ITAM1 and the remaining CD3zeta domain is deleted (residues 90-164). As another example, ITAM1 is substituted with the amino acid sequence of ITAM3, and the remaining CD3zeta domain is deleted (residues 90-164). See, for example, Bridgeman et al., Clin. Exp. Immunol. 175(2): 258-67 (2014); Zhao et al., J. Immunol. 183(9): 5563-74 (2009); Maus et al., WO-2018/132506; Sadelain et al., WO-2019/133969, Feucht et al., Nat Med. 25(1): 82-88 (2019).

Thus, in some aspects, the antigen binding molecule is linked to one or more cell signaling modules. In some embodiments, cell signaling modules include CD3 transmembrane domain, CD3 intracellular signaling domains, and/or other CD transmembrane domains. The CAR can also further include a portion of one or more additional molecules such as Fc receptor g, CD8, CD4, CD25, or CD16.

In some embodiments, upon ligation of the CAR, the cytoplasmic domain or intracellular signaling domain of the CAR activates at least one of the normal effector functions or responses of the corresponding non-engineered immune cell (typically a T cell). For example, the CAR can induce a function of a T cell such as cytolytic activity or T-helper activity, secretion of cytokines or other factors.

The CAR or other antigen-specific receptor can also be an inhibitory CAR (e.g. iCAR) and includes intracellular components that dampen or suppress a response, such as an immune response. Examples of such intracellular signaling components are those found on immune checkpoint molecules, including PD-1, CTLA4, LAG3, BTLA, OX2R, TIM-3, TIGIT, LAIR-1, PGE2 receptors, or EP2/4 Adenosine receptors including A2AR. In some aspects, the engineered cell includes an inhibitory CAR including a signaling domain of or derived from such an inhibitory molecule, such that it serves to dampen the response of the cell. Such CARs are used, for example, to reduce the likelihood of off-target effects when the antigen recognized by the activating receptor, e.g, CAR, is also expressed, or may also be expressed, on the surface of normal cells.

### TCRs

In some embodiments, the antigen-specific receptors include recombinant modified T cell receptors (TCRs) and/or TCRs cloned from naturally occurring T cells. Nucleic acid encoding the TCR can be obtained from a variety of sources, such as by polymerase chain reaction (PCR) amplification of naturally occurring TCR DNA sequences, followed by expression of antibody variable regions, followed by selecting for specific binding to antigen. In some embodiments, the TCR is obtained from T-cells isolated from a patient, or from cultured T-cell hybridomas. In some embodiments, the TCR clone for a target antigen has been generated in transgenic mice engineered with human immune system genes (e.g., the human leukocyte antigen system, or HLA). Sec, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354.

A “T cell receptor” or “TCR” refers to a molecule that contains a variable alpha and beta chains (also known as TCRα and TCRβ, respectively) or a variable gamma and delta chains (also known as TCRγ and TCRδ, respectively) and that is capable of specifically binding to an antigen peptide bound to a MHC receptor. In some embodiments, the antigen-binding domain of the TCR binds its target antigen with KD affinity of about 1×10⁻⁷ or less, about 5×10⁻⁸ or less, about 1×10⁻⁸ or less, about 5×10⁻⁹ or less, about 1×10⁻⁹ or less, about 5×10⁻¹⁰ or less, about 1×10⁻¹⁰ or less, about 5×10⁻¹¹ or less, about 1×10⁻¹¹ or less, about 5×10⁻¹² or less, or about 1×10⁻¹² or less (lower numbers indicating higher affinity). In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et ah, Immunobiology: The Immune System in Health and Disease, 3rd Ed., Current Biology Publications, p. 4:33, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length modified TCRs, including TCRs in the αβ form or γδ form. The term “TCR” also includes a TCR modified to include a VH and/or VL of an antibody.

Thus, for purposes herein, reference to a TCR includes any modified TCR or functional fragment thereof, such as an antigen-binding portion of a TCR that binds to a specific antigenic peptide bound in an MHC molecule, i.e. MHC-peptide complex. An “antigen binding portion” or “antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable alpha chain and variable beta chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., Proc. Nat'l. Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the beta chain can contain a further hypervariability (HV4) region.

In some embodiments, the TCR chains contain a constant domain. For example, like immunoglobulins, the extracellular portion of TCR chains (e.g., α-chain, β-chain) can contain two immunoglobulin domains, a variable domain {e.g., Vα or Vβ; typically amino acids 1 to 116 based on Kabat numbering Kabat et al., Sequences of Proteins of Immunological Interest, US Dept. Health and Human Services, Public Health Service National Institutes of Health, 1991, 5th ed.) at the N-terminus, and one constant domain (e.g., α-chain constant domain or Cα or TRAC, typically amino acids 117 to 259 based on Kabat, β-chain constant domain or Cβ or TRBC, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contain a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (gamma (γ), delta (δ), and epsilon (ε)) and the zeta-chain. For example, in mammals the complex can contain a CD3gamma chain, a CD3delta chain, two CD3epsilon chains, and a homodimer of CD3zeta chains. The CD3gamma chains are highly related cell surface proteins of the immunoglobulin superfamily containing a single immunoglobulin domain. The transmembrane regions of the CD3gamma, CD3delta, and CD3epsilon chains are negatively charged, which is a characteristic that allows these chains to associate with the positively charged T cell receptor chains and play a role in propagating the signal from the TCR into the cell. The intracellular tails of the CD3gamma, CD3delta, and CD3epsilon chains each contain a single conserved motif known as an immunoreceptor tyrosine-based activation motif or ITAM, whereas each CD3 zeta chain has three ITAMs. Generally, ITAMs are involved in the signaling capacity of the TCR complex. The CD3gamma, delta, epsilon and zeta chains together form what is known as the T cell receptor complex.

Modified TCR of the disclosure can comprise a heterologous antigen-binding domain and a native TCR constant domain (alpha or beta) or fragment thereof, wherein the modified TCR is capable of activating a CD3 zeta polypeptide. Example modified TCR, designated HI-TCR or HIT-CAR herein, comprise (a) a first antigen-binding chain comprising an antigen-binding fragment of a heavy chain variable region (VH) of an antibody; and (b) a second antigen-binding chain comprising an antigen-binding fragment of a light chain variable region (VL) of an antibody; wherein the first and second antigen-binding chains each comprise a native or variant TRAC (constant region) or fragment thereof, or a native or variant TRBC (constant region) or fragment thereof. In some embodiments, at least one of the TRAC polypeptide and the TRBC polypeptide is endogenous, typically the TRAC polypeptide, and optionally one or both of the endogenous TRAC and TRBC polypeptides is inactivated.

In some designs, the HI-TCR comprises (a) a chimeric TCR alpha chain comprising a VH or fragment thereof fused to a native or variant TRAC or fragment thereof, optionally in which a amino acids of the VH (or TRAC) are removed, and (b) a chimeric TCR beta chain comprising a VL or fragment thereof fused to a native or variant TRBC or fragment thereof, optionally in which amino acids of the VL (or TRBC) are removed. In other designs, the HI-TCR comprises (a) a chimeric TCR alpha chain comprising a VL or fragment thereof fused to a native or variant TRAC or fragment thereof, optionally in which amino acids of the VL (or TRAC) are removed, and (b) a chimeric TCR beta chain comprising a VH or fragment thereof fused to a native or variant TRBC or fragment thereof, optionally in which amino acids of the VH (or TRBC) are removed. In yet other designs, the HI-TCR comprises just a VH or fragment thereof fused to a native or variant TRAC or fragment thereof, or fused to a native or variant TRBC or fragment thereof, optionally in which amino acids of the VH (or TRAC or TRBC) are removed. HI-TCR (HIT-CAR) are described in Int'l Pat. Pub. No. WO 2019/157454, incorporated by reference herein in its entirety. Yet other modified TCRs are disclosed in Int'l Pat. Pub. No. WO 2018/067993, incorporated herein by reference in its entirety, and in Bacuerle, et al. Synthetic TRUC receptors engaging the complete T cell receptor for potent anti-tumor response. Nat Commun 10, 2087 (2019). For example, any one or more, or two or more, of the alpha, beta, gamma or epsilon chains may be fused to an antibody variable region, e.g., VH and/or VL such as an scFv.

In some embodiments, the nucleic acid encoding the heterologous antigen-binding domain (e.g., VH or variant or fragment thereof, or VL or variant or fragment thereof) is inserted into the endogenous TRAC locus and/or TRBC locus of the immune cell. Optionally, the nucleic acid encoding the chimeric TCR alpha (or beta) chain is operatively linked to an endogenous promoter of the T-cell receptor such that its expression is under control of the endogenous promoter. The insertion of the nucleic acid sequence can also inactivate or disrupt the endogenous expression of a TCR comprising a native TCR alpha chain and/or a native TCR beta chain. The insertion of the nucleic acid sequence may reduce endogenous TCR expression by at least about 75%, 80%, 85%, 90% or 95%.

The immune cells comprising the recombinant TCR typically provide superior activity when the antigen has a low density on the cell surface of less than about 10,000 molecules per cell, e.g. less than about 5,000, 4,000, 3,000, 2,000, 1,000, 500, 250 or 100 molecules per cell. In some embodiments, the antigen is expressed at low density by the target cell, e.g., less than about 6,000 molecules of the target antigen per cell. In some embodiments, the antigen is expressed at a density of less than about 5,000 molecules, less than about 4,000 molecules, less than about 3,000 molecules, less than about 2,000 molecules, less than about 1,000 molecules, or less than about 500 molecules of the target antigen per cell. In some embodiments, the antigen is expressed at a density of less than about 2,000 molecules, such as e.g., less than about 1,800 molecules, less than about 1,600 molecules, less than about 1,400 molecules, less than about 1,200 molecules, less than about 1,000 molecules, less than about 800 molecules, less than about 600 molecules, less than about 400 molecules, less than about 200 molecules, or less than about 100 molecules of the target antigen per cell. In some embodiments, the antigen is expressed at a density of less than about 1,000 molecules, such as e.g., less than about 900 molecules, less than about 800 molecules, less than about 700 molecules, less than about 600 molecules, less than about 500 molecules, less than about 400 molecules, less than about 300 molecules, less than about 200 molecules, or less than about 100 molecules of the target antigen per cell. In some embodiments, the antigen is expressed at a density ranging from about 5,000 to about 100 molecules of the target antigen per cell, such as e.g., from about 5,000 to about 1,000 molecules, from about 4,000 to about 2,000 molecules, from about 3,000 to about 2,000 molecules, from about 4,000 to about 3,000 molecules, from about 3,000 to about 1,000 molecules, from about 2,000 to about 1,000 molecules, from about 1,000 to about 500 molecules, from about 500 to about 100 molecules of the target antigen per cell. In some embodiments, the recombinant TCR T cell therapy targets an antigen that is expressed at low density compared to a density in a wild-type cell.

Other examples of antigen-specific receptors, including CARs and recombinant modified TCRs, as well as methods for engineering and introducing the receptors into cells, include those described, for example, in international patent application publication numbers WO-2000/014257, WO-2013/126726, WO-2012/129514, WO-2014/031687, WO-2013/166321, WO-2013/071154, WO-2013/123061 U.S. patent application publication numbers US-2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLOS ONE 8(4): c61338; Turtle et al., Curr. Opin. Immunol., 2012 October; 24(5): 633-39; Wu et al., Cancer, 2012 Mar. 18(2): 160-75. In some aspects, the antigen-specific receptors include a CAR as described in U.S. Pat. No. 7,446,190, and those described in International Patent Application Publication No.: WO-2014/055668 A1.

### Co-Stimulatory Receptors

The cells of the disclosure with modified SUV39H1 expression may further comprise at least one or at least two exogenous co-stimulatory ligands. Co-stimulatory ligands include CD80, CD86, 4-1BBL, CD275, CD40L, OX40L or any combination thereof. In some embodiments, the co-stimulatory ligand is CD80 or 4-1BBL.

In some embodiments, the cells comprise at least one or at least two co-stimulatory receptors. Such co-stimulatory receptors include chimeric receptors comprising a co-stimulatory ligand fused to at least one or at least two co-stimulatory molecule(s). Co-stimulatory ligands include CD80, CD86, 4-1BBL, CD275, CD40L, OX40L or any combination thereof. In some embodiments, the co-stimulatory ligand is CD80 or 4-1BBL. Example co-stimulatory molecules are CD28, 4-1BB, OX40, ICOS, DAP-10, CD27, CD40, NKG2D, CD2, or any combination thereof. In some embodiments, the chimeric receptor comprises a first co-stimulatory molecule that is 4-1BB and a second co-stimulatory molecule that is CD28.

In some embodiments, the cell comprises a co-stimulatory receptor comprising the extracellular domain of CD80, transmembrane domain of CD80, and an intracellular 4-1BB domain. Example co-stimulatory ligands, molecules and receptors (or fusion polypeptides) are described in Int'l Pat. Pub. No. WO-2021/016174, incorporated by reference herein in its entirety.

The cells of the disclosure with modulated SUV39H1 expression may also comprise T-cell specific engagers, such as BiTEs, or bispecific antibodies that bind not only the desired antigen but also an activating T-cell antigen such as CD3 epsilon. In some embodiments, the BiTe comprises an antigen-binding domain, e.g. scFv, linked to a T-cell recognizing domain, e.g., heavy variable domain and/or light variable domain of an anti-CD3 antibody.

### Antigens

Antigens include antigens associated with diseases or disorders, including proliferative, neoplastic, and malignant diseases and disorders, more particularly cancers. Infectious diseases and autoimmune, inflammatory or allergic diseases are also contemplated.

The cancer may be a solid cancer or a “liquid tumor” such as cancers affecting the blood, bone marrow and lymphoid system, also known as tumors of the hematopoietic and lymphoid tissues, which notably include leukemia and lymphoma. Liquid tumors include for example acute myelogenous leukemia (AML), chronic myelogenous leukemia (CML), acute lymphocytic leukemia (ALL), and chronic lymphocytic leukemia (CLL), (including various lymphomas such as mantle cell lymphoma, non-Hodgkins lymphoma (NHL), adenoma, squamous cell carcinoma, laryngeal carcinoma, gallbladder and bile duct cancers, cancers of the retina such as retinoblastoma).

Solid cancers notably include cancers affecting one of the organs selected from the group consisting of colon, rectum, skin, endometrium, lung (including non-small cell lung carcinoma), uterus, bones (such as Osteosarcoma, Chondrosarcomas, Ewing's sarcoma, Fibrosarcomas, Giant cell tumors, Adamantinomas, and Chordomas), liver, kidney, esophagus, stomach, bladder, pancreas, cervix, brain (such as Meningiomas, Glioblastomas, Lower-Grade Astrocytomas, Oligodendrocytomas, Pituitary Tumors, Schwannomas, and Metastatic brain cancers), ovary, breast, head and neck region, testis, prostate and the thyroid gland.

Cancers include cancers affecting the blood, bone marrow and lymphoid system as described above. In some embodiments, the cancer is, or is associated, with multiple myeloma. Antigens associated with multiple myeloma include CD38, CD138, and/or CS-1. Other exemplary multiple myeloma antigens include CD56, TIM-3, CD33, CD123, and/or CD44.

Diseases also encompass infectious diseases or conditions, such as, but not limited to, viral, retroviral, bacterial, protozoal or parasitic, infections, HIV immunodeficiency, Cytomegalovirus (CMV), Epstein-Barr virus (EBV), adenovirus, BK polyomavirus. Viral antigens include antigens of HIV, HCV, HBV.

In some embodiments the extracellular antigen-binding domain binds to any of the tumor neoantigenic peptides disclosed in Int'l Pat. Pub. No. WO 2021/043804, incorporated by reference herein in its entirety. For example, the antigen-binding domain binds to any of the peptides of SEQ ID NO: 1-117 or to a neoantigenic peptide comprising at least 8, 9, 10, 11 or 12 amino acids that is encoded by a part of an open reading frame (ORF) of any of the fusion transcript sequences of any one of SEQ ID NO: 118-17492 of WO 2021/043804.

Diseases also encompass autoimmune or inflammatory diseases or conditions, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease multiple sclerosis, asthma, and/or diseases or conditions associated with transplant. In such circumstances, a T-regulatory cell may be the cell in which SUV39H1 is inhibited.

In some embodiments, the antigen is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells. In some such embodiments, a multi-targeting and/or gene disruption approach as provided herein is used to improve specificity and/or efficacy.

In some embodiments, the antigen is a universal tumor antigen. The term “universal tumor antigen” refers to an immunogenic molecule, such as a protein, that is, generally, expressed at a higher level in tumor cells than in non-tumor cells and also is expressed in tumors of different origins. In some embodiments, the universal tumor antigen is expressed in more than 30%, 40%, 50%, 60%, 70%, 75%, 80%, 85%, 90% or more of human cancers. In some embodiments, the universal tumor antigen is expressed in at least three, at least four, at least five, at least six, at least seven, at least eight or more different types of tumors. In some cases, the universal tumor antigen may be expressed in non-tumor cells, such as normal cells, but at lower levels than it is expressed in tumor cells. In some cases, the universal tumor antigen is not expressed at all in non-tumor cells, such as not expressed in normal cells. Exemplary universal tumor antigens include, for example, human telomerase reverse transcriptase (hTERT), survivin, mouse double minute 2 homolog (MDM2), cytochrome P450 1 B1 (CYP1 B), HER2/neu, p95HER2, Wilms tumor gene 1 (WT1), livin, alphafetoprotein (AFP), carcinoembryonic antigen (CEA), mucin 16 (MUC16), MUC1, prostate-specific membrane antigen (PSMA), p53 or cyclin (D1). Peptide epitopes of tumor antigens, including universal tumor antigens, are known in the art and, in some aspects, can be used to generate MHC-restricted antigen-specific receptors, such as TCRs or TCR-like CARs (see e.g. published PCT application No. WO-2011/009173 or WO-2012/135854 and published U.S. application No. US20140065708).

In some embodiments, the cancer is, or is associated, with overexpression of HER2 or p95HER2. p95HER2 is a constitutively active C-terminal fragment of HER2 that is produced by an alternative initiation of translation at methionine 611 of the transcript encoding the full-length HER2 receptor. The amino acid sequence of p95HER2 is set forth in SEQ ID NO: 11, and the amino acid sequence of the extracellular domain is SEQ ID NO: 12

HER2 or p95HER2 has been reported to be overexpressed in breast cancer, as well as gastric (stomach) cancer, gastroesophageal cancer, esophageal cancer, ovarian cancer, uterine endometrial cancer, cervix cancer, colon cancer, bladder cancer, lung cancer, and head and neck cancers. Patients with cancers that express the p95HER2 fragment have a greater probability of developing metastasis and a worse prognosis than those patients who mainly express the complete form of HER2. Saez et al., Clinical Cancer Research, 12:424-431 (2006).

Other antigens include orphan tyrosine kinase receptor ROR1, tEGFR, Her2, p95HER2, LI-CAM, CD19, CD20, CD22, mesothelin, CEA, Claudin 18.2, hepatitis B surface antigen, anti-folate receptor, CD23, CD24, CD30, CD33, CD38, CD44, CD70, EGFR, EGP-2, EGP-4, EPHa2, ErbB2, 3, or 4, FBP, FcRH5 fetal acetylcholine e receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp1OO, oncofetal antigen, ROR1, TAG72, VEGF-R2, carcinoembryonic antigen (CEA), prostate specific antigen, PSMA, Her2/neu, p95HER2, estrogen receptor, progesterone receptor, ephrinB2, CD 123, CS-1, c-Met, GD-2, and MAGE A3, CE7, Wilms Tumor 1 (WT-1), a cyclin, such as cyclin A1 (CCNA1), and/or molecules expressed by HIV, HCV, HBV or other pathogens.

In some embodiments, recombinant nucleic acids encoding antigen receptors as previously described are transferred into T cells via electroporation {see, e.g., Chicaybam et al, (2013) PLOS ONE 8(3): c60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, c74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346:776-777 (1990)); and strontium phosphate DNA co-precipitation (Brash et al., Mol. Cell Biol., 7:2031-2034 (1987)).

Particularly useful vectors for generating a target construct that provides transgene vectorization for homologous recombination-mediated targeting include, but are not limited to, recombinant Adeno-Associated Virus (rAAV), recombinant non-integrating lentivirus (rNILV), recombinant non-integrating gamma-retrovirus (rNIgRV), single-stranded DNA (linear or circular), and the like. Such vectors can be used to introduce a transgene into an immune cell of the invention by making a targeting construct (see, for example, Miller, Hum. Gene Ther. 1(1): 5-14 (1990); Friedman, Science 244:1275-1281 (1989); Eglitis et al., BioTechniques 6:608-614 (1988); Tolstoshev et al., Current Opin. Biotechnol. 1:55-61 (1990); Sharp, Lancet 337:1277-1278 (1991); Cornetta et al., Prog. Nucleic Acid Res. Mol. Biol. 36:311-322 (1989); Anderson, Science 226:401-409 (1984); Moen, Blood Cells 17:407-416 (1991); Miller et al., Biotechnology 7:980-990 (1989); Le Gal La Salle et al., Science 259:988-990 (1993); and Johnson, Chest 107:77S-83S (1995); Rosenberg et al., N. Engl. J. Med. 323:370 (1990); Anderson et al., U.S. Pat. No. 5,399,346; Scholler et al., Sci. Transl. Med. 4:132-153 (2012; Parente-Pereira et al., J. Biol. Methods 1(2): e7 (1-9) (2014); Lamers et al., Blood 117(1): 72-82 (2011); Reviere et al., Proc. Natl. Acad. Sci. USA 92:6733-6737 (1995); Wang et al., Gene Therapy 15: 1454-1459 (2008)).

In some embodiments, the exogenous nucleic acid or the targeting construct comprises a 5′ homology arm and a 3′ homology arm to promote recombination of the nucleic acid sequence into the cell genome at the nuclease cleavage site.

In some embodiments, an exogenous nucleic acid can be introduced into the cell using a single-stranded DNA template. The single-stranded DNA can comprise the exogenous nucleic acid and, in preferred embodiments, can comprise 5′ and 3′ homology arms to promote insertion of the nucleic acid sequence into the nuclease cleavage site by homologous recombination. The single-stranded DNA can further comprise a 5′ AAV inverted terminal repeat (ITR) sequence 5′ upstream of the 5′ homology arm, and a 3′ AAV ITR sequence 3′ downstream of the 3′ homology arm. In other particular embodiments, the targeting construct comprises in 5′ to 3′ order: a first viral sequence, a left homology arm, a nucleic acid sequence encoding an element that create polycistronic expression cassette (e.g. various viral and non-viral Internal Ribosome Entry Sites (IRES, e.g., FGF-1 IRES, FGF-2 IRES, VEGF IRES, IGF-II IRES, NF-kB IRES, RUNX1 IRES, p53 IRES, hepatitis A IRES, hepatitis C IRES, pestivirus IRES, aphthovirus IRES, picomavirus IRES, poliovirus IRES and encephalomyocarditis virus IRES) and cleavable linkers (e.g, 2 A peptides, e.g., P2A, T2A, E2A and F2A peptides), preferably a cleavable linker), a transgene, a polyadenylation sequence, a right homology arm and a second viral sequence. In a preferred embodiment, the targeting construct comprises in 5′ to 3′ order: a first viral sequence, a left homology arm, a nucleic acid sequence encoding a self-cleaving linker (such as the porcine teschovirus 2A), a nucleic acid sequence encoding a CAR or a modified TCR (e.g. a Hi-CTR), a polyadenylation sequence, a right homology arm and a second viral sequence. Another suitable targeting construct can comprise sequences from an integrative-deficient Lentivirus (see, for example, Wanisch et al., Mol. Ther. 17(8): 1316-1332 (2009)).

In some embodiments, the viral nucleic acid sequence comprises sequences of an integrative-deficient Lentivirus. It is understood that any suitable targeting construction compatible with a homologous recombination system employed can be utilized. The AAV nucleic acid sequences that function as part of a targeting construct can be packaged in several natural or recombinant AAV capsids or particles. In a particular embodiment, the AAV particle is AAV6. In a particular embodiment, an AAV2-based targeting construct is delivered to the target cell using AAV6 viral particles. In a particular embodiment, the AAV sequences are AAV2, AAV5 or AAV6 sequences.

In some embodiments, the gene encoding an exogenous nucleic acid sequence of the invention can be introduced into the cell by transfection with a linearized DNA template. In some examples, a plasmid DNA encoding an exogenous nucleic acid sequence can include nuclease cleavage site (such as class II, type II, V or VI Cas nuclease) at both sides of the left homology arm such that the circular plasmid DNA is linearized and allows precise in-frame integration of exogenous DNA without backbone vector sequences (see for example Hisano Y, Sakuma T, Nakade S, et al. Precise in-frame integration of exogenous DNA mediated by CRISPR/Cas9 system in zebrafish. Sci Rep. 2015; 5:8841).

In some embodiments, the vector incorporates an endogenous promoter such as a TCR promoter. Such a vector could provide for expression in a manner similar to that provided by an endogenous promoter, such as a TCR promoter. Such a vector can be useful, for example, if the site of integration does not provide for efficient expression of a transgene, or if disruption of the endogenous gene controlled by the endogenous promoter would be detrimental to the T cell or would result in a decrease in its effectiveness in T cell therapy. In a preferred embodiment, such a vector can be useful, for example, if the site of integration does not provide for efficient expression of nucleic acid sequence encoding a CAR or a modified TCR. The promoter can be an inducible promoter or a constitutive promoter. Expression of a nucleic acid sequence under the control of an endogenous or vector-associated promoter occurs under suitable conditions for the cell to express the nucleic acid, for example, growth conditions, or in the presence of an inducer with an inducible promoter, and the like. Such conditions are well understood by those skilled in the art.

The targeting construct can optionally be designed to include an element that create polycistronic expression cassette (including but not limited to, various viral and non-viral Internal Ribosome Entry Sites (IRES, e.g., FGF-1 IRES, FGF-2 IRES, VEGF IRES, IGF-II IRES, NF-kB IRES, RUNX1 IRES, p53 IRES, hepatitis A IRES, hepatitis C IRES, pestivirus IRES, aphthovirus IRES, picomavirus IRES, poliovirus IRES and encephalomyocarditis virus IRES) and cleavable linkers (e.g, 2 A peptides, e.g., P2A, T2A, E2A and F2A peptides)) directly upstream of the nucleic acid sequences encoding the transgene. In preferred embodiments, the targeting construct can optionally be designed to include a cleavable linked (e.g.: P2A, T2A, etc.) sequence directly upstream of the nucleic acid sequences encoding a therapeutic protein (e.g. an engineered antigen receptor). P2A and T2A are self-cleaving peptide sequences, which can be used for bicistronic or multicistronic expression of protein sequences (see Szymczak et al., Expert Opin. Biol. Therapy 5(5): 627-638 (2005)).

Well-suited AAV constructs for HIT expressing in an immunoresponsive cell according to the present application are for example described in Mansilla-Soto, J., Eyquem, J., Haubner, S. et al. HLA-independent T cell receptors for targeting tumors with low antigen density. Nat Med 28, 345-352 (2022) and have been used in the results included herein, notably for the in vivo experiments.

Typical well-suited constructs typically include a TRBC or TRAC sequence (which can be a native or modified TRBC or TRAC sequence, including murine sequences as described herein), a cleavable linker sequence (as defined above, but such as a 2A sequence), a TRAC or TRBC sequence (which can be a native or modified TRBC or TRAC sequence, including murine sequences as described herein). The TRBC and/or the TRAC sequence is typically fused (preferably in 5′) to a sequence coding for an antibody fragment as above described (e.g. a VH, a VH, an scFv, a single domain antibody, a VHH, etc.). In some embodiment, the booster (co-stimulatory ligand) sequence is included in the construct, such that in preferred embodiments, the construct further includes a cleavable linker sequence (e.g. a 2A sequence) and a booster (co-stimulatory ligand) sequence. Typically, the TRAC or TRBC sequence in the 3′ end of the construct is fused to cleavable linker which is also fused to the booster (co-stimulatory ligand and or costimulatory receptor CCR) sequence (see FIG. 11, as well as FIGS. 29-30). The booster ((co-stimulatory ligand and/or costimulatory receptor CCR) sequence can be any one as herein described and can be notably a CD80 sequence or a CD80_4-1BB sequence as herein described (see for example SEQ ID NO:32-33 and 52-53).

If desired, the targeting construct can optionally be designed to include a reporter, for example, a reporter protein that provides for identification of transduced cells. Exemplary reporter proteins include, but are not limited to, fluorescent proteins, such as mCherry, green fluorescent protein (GFP), blue fluorescent protein, for example, EBFP, EBFP2, Azurite, and mKalamal, cyan fluorescent protein, for example, ECFP, Cerulean, and CyPet, and yellow fluorescent protein, for example, YFP, Citrine, Venus, and YPet. Typically, the targeting construct comprises a polyadenylation (poly A) sequence 3′ of the transgene. In a preferred embodiment, the targeting construct comprises a polyadenylation (poly A) sequence in 3′ of the nucleic acid sequences encoding a CAR and/or a modified TCR (e.g. a Hi)-TCR).

### Therapeutic Uses

The cells, modified oligonucleotides, nucleic acids, or vectors of the disclosure may be used in adoptive cell therapy (notably adoptive T cell therapy or adoptive NK cell therapy). In some embodiments, the use is in the treatment of cancer in a subject in need thereof, but uses also include the treatment of infectious diseases and autoimmune, inflammatory or allergic diseases. In some embodiments, the subject is suffering from a cancer or at risk of suffering from a cancer. The modified oligonucleotides, nucleic acids, or vectors of the disclosure are optionally within a delivery vehicle or composition as disclosed herein, including a liposome, lipid-containing complex, nanoparticle, gold particle, or polymer complex. Such compositions may further comprise stabilizing agents or transfection facilitating agents such as surface active agents, quinone analogs, vesicles such as squalene and squalene, hyaluronic acid, lipids, liposomes, lecithin liposomes, calcium ions, viral proteins, polyanions, polycations, including poly-L-glutamate, or nanoparticles, gold particles, or other known agents.

In such methods, a subject is administered one or more cells, modified oligonucleotides, nucleic acids, or vectors described herein to a subject in need thereof, in an amount effective to treat the disease or disorder. For example, cells expressing one or more antigen-specific receptors are administered at a dose effective to treat the disease or disorder associated with the antigen(s). Treatment of any of the diseases listed above under the “Antigen” section is contemplated.

In some embodiments, an immune cell expressing an antigen receptor (e.g a Hi T cell antigen receptor) as herein described can be used for the treatment of a patient having a median value of less than less than about 6,000 molecules of the target antigen per cell. In some embodiments, the antigen is expressed at a density (typically median value) of less than about 5,000 molecules, less than about 4,000 molecules, less than about 3,000 molecules, less than about 2,000 molecules, less than about 1,000 molecules, or less than about 500 molecules of the target antigen per cell. In some embodiments, the antigen is expressed at a density (typically median value) of less than about 2,000 molecules, such as e.g., less than about 1,800 molecules, less than about 1,600 molecules, less than about 1,400 molecules, less than about 1,200 molecules, less than about 1,000 molecules, less than about 800 molecules, less than about 600 molecules, less than about 400 molecules, less than about 200 molecules, or less than about 100 molecules of the target antigen per cell. In some embodiments, the antigen is expressed at a density of less than about 1,000 molecules, such as e.g., less than about 900 molecules, less than about 800 molecules, less than about 700 molecules, less than about 600 molecules, less than about 500 molecules, less than about 400 molecules, less than about 300 molecules, less than about 200 molecules, or less than about 100 molecules of the target antigen per cell. In some embodiments, the antigen is expressed at a density ranging from about 5,000 to about 100 molecules of the target antigen per cell, such as e.g., from about 5,000 to about 1,000 molecules, from about 4,000 to about 2,000 molecules, from about 3,000 to about 2,000 molecules, from about 4,000 to about 3,000 molecules, from about 3,000 to about 1,000 molecules, from about 2,000 to about 1,000 molecules, from about 1,000 to about 500 molecules, from about 500 to about 100 molecules of the target antigen per cell. Quantification of the target antigen density per cell can be achieved as described in Jasper, G. A., Arun, I., Venzon, D., Kreitman, R. J., Wayne, A. S., Yuan, C. M., Marti, G. E., & Stetler-Stevenson, M. (2011). Variables affecting the quantitation of CD22 in neoplastic B cells. Cytometry. Part B, Clinical cytometry, 80(2), 83-90.

The cells may be administered at certain doses. For example, the immune cells (e.g., T cells or NK cells) in which SUV39H1 has been inhibited may be administered to adults at doses of less than about 10⁸ cells, less than about 5×10⁷ cells, less than about 10⁷ cells, less than about 5×10⁶ cells, less than about 10⁶ cells, less than about 5×10⁵ cells or less than about 10⁵ cells. The dose for pediatric patients may be about 100-fold less. In alternative embodiments, any of the immune cells (e.g. T-cells) described herein may be administered to patients at doses ranging from about 10⁵ to about 10⁹ cells, or about 10⁵ to about 10⁸ cells, or about 10⁵ to about 10⁷ cells, or about 10⁶ to about 10⁸ cells.

The subject (i.e. patient) is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent. In some examples, the patient or subject is a validated animal model for disease, adoptive cell therapy, and/or for assessing toxic outcomes such as cytokine release syndrome (CRS). In some embodiments, said subject has a cancer, is at risk of having a cancer, or is in remission of a cancer.

In some embodiments, the cells or compositions are administered to the subject, such as a subject having or at risk for a cancer or any one of the diseases as mentioned above. In some aspects, the methods thereby treat, e.g., ameliorate one or more symptom of, the disease or condition, such as with reference to cancer, by lessening tumor burden in a cancer expressing an antigen recognized by the engineered cell.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10): 577-85). Sec, e.g., Themeli et al. (2013) Nat Biotechnol. 31 (10): 928-933; Tsukahara et al. (2013) Biochem Biophys Res Commun 438(1): 84-9; Davila et al. (2013) PLOS ONE 8(4): e61338.

Administration of at least one cell according to the disclosure to a subject in need thereof may be combined with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cell populations are administered prior to the one or more additional therapeutic agents. In some embodiments, the cell populations are administered after to the one or more additional therapeutic agents.

With reference to cancer treatment, a combined cancer treatment can include but is not limited to cancer chemotherapeutic agents, cytotoxic agents, hormones, anti-angiogens, radiolabelled compounds, immunotherapy, surgery, cryotherapy, and/or radiotherapy.

Conventional cancer chemotherapeutic agents include alkylating agents, antimetabolites, anthracyclines, topoisomerase inhibitors, microtubule inhibitors and B-raf enzyme inhibitors.

Alkylating agents include the nitrogen mustards (such as mechlorethamine, cyclophosphamide, ifosfamide, melphalan and chlorambucil), ethylenamine and methylenamine derivatives (such as altretamine, thiotepa), alkyl sulfonates (such as busulfan), nitrosoureas (such as carmustine, lomustine, estramustine), triazenes (such as dacarbazine, procarbazine, temozolomide), and platinum-containing antineoplastic agents (such as cisplatin, carboplatin, oxaliplatin).

Antimetabolites include 5-fluorouracil (5-FU), 6-mercaptopurine (6-MP), Capecitabine (Xeloda®), Cytarabine (Ara-C®), Floxuridine, Fludarabine, Gemcitabine (Gemzar®), Hydroxyurea, Methotrexate, Pemetrexed (Alimta®).

Anthracyclines include Daunorubicin, Doxorubicin (Adriamycin®), Epirubicin. Idarubicin. Other anti-tumor antibiotics include Actinomycin-D, Bleomycin, Mitomycin-C, Mitoxantrone.

Topoisomerase inhibitors include Topotecan, Irinotecan (CPT-11), Etoposide (VP-16), Teniposide or Mitoxantrone.

Microtubule inhibitors include Estramustine, Ixabepilone, the taxanes (such as Paclitaxel, Docetaxel and Cabazitaxel), and the vinca alkaloids (such as Vinblastine, Vincristine, Vinorelbine, Vindesine and Vinflunine)

B-raf enzyme inhibitors include vemurafenib (Zelboraf), dabrafenib (Tafinlar), and encorafenib (Braftovi).

Immunotherapy includes but is not limited to immune checkpoint modulators (i.e. inhibitors and/or agonists), cytokines, immunomodulating monoclonal antibodies, cancer vaccines.

Preferably, administration of cells in an adoptive T cell therapy according to the disclosure is combined with administration of immune checkpoint modulators. Examples include inhibitors of (e.g. antibodies that bind specifically to and inhibit activity of) PD-1, CTLA4, LAG 3, BTLA, OX2R, TIM-3, TIGIT, LAIR-1, PGE2 receptors, and/or EP2/4 Adenosine receptors including A2AR. Preferably, the immune checkpoint modulators comprise anti-PD-1 and/or anti-PDL-1 inhibitors (e.g., anti-PD-1 and/or anti-PDL-1 antibodies).

The present disclosure also relates to the use of a composition comprising the cells as herein described for the manufacture of a medicament for treating a cancer, an infectious disease or condition, an autoimmune disease or condition, or an inflammatory disease or condition in a subject.

### EXAMPLES

### Example 1: Expression of lncRNA AF196970.3 in Human Tissues

Expression of lncRNA AF196970.3 was examined in various human tissues.

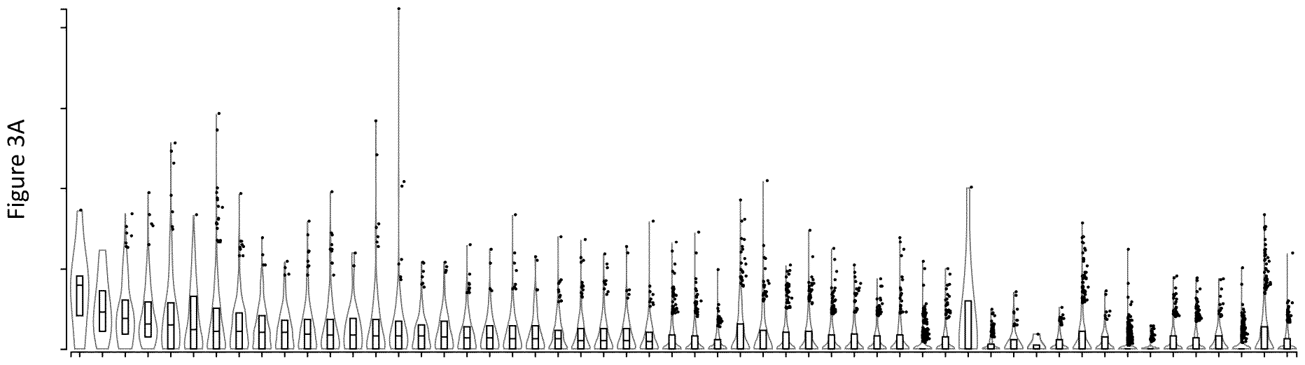

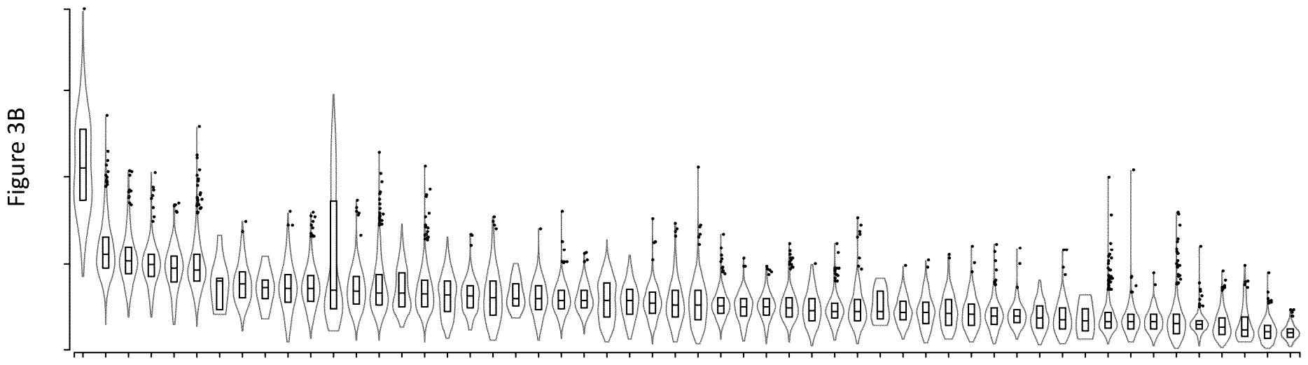

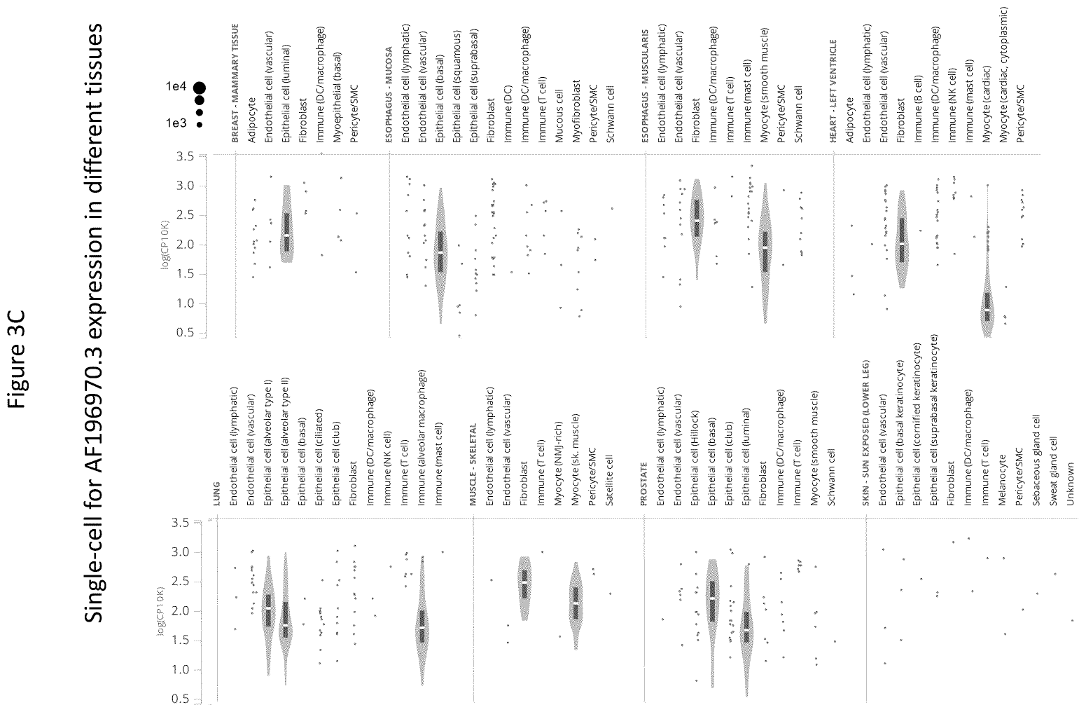

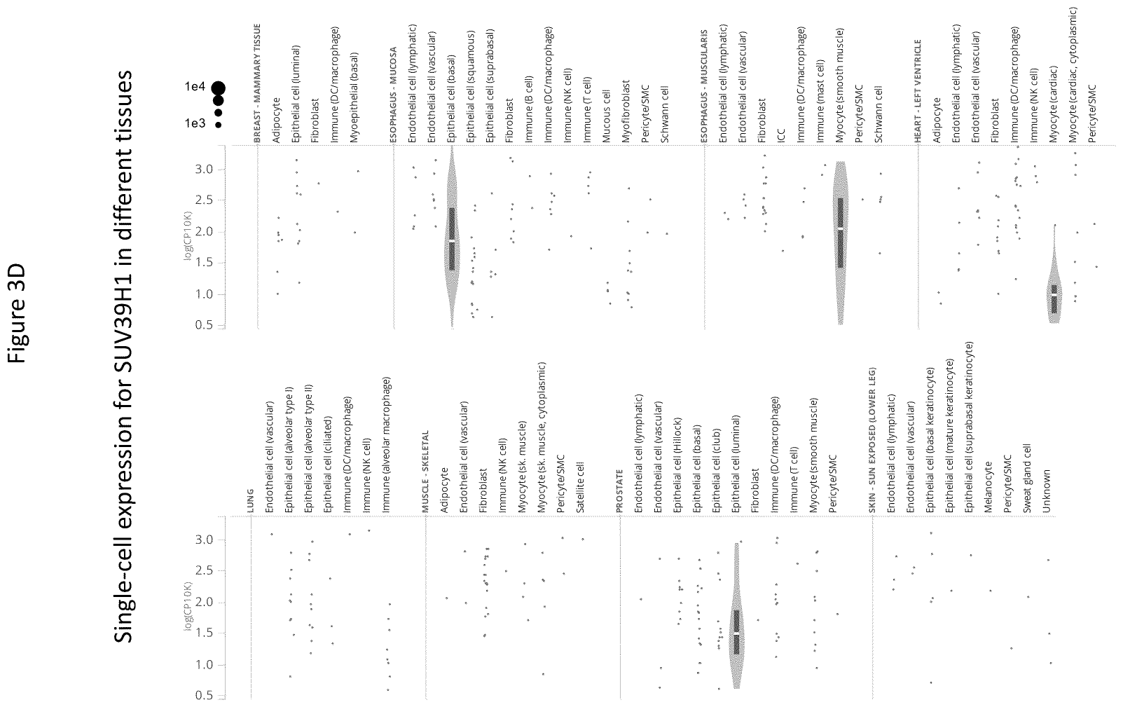

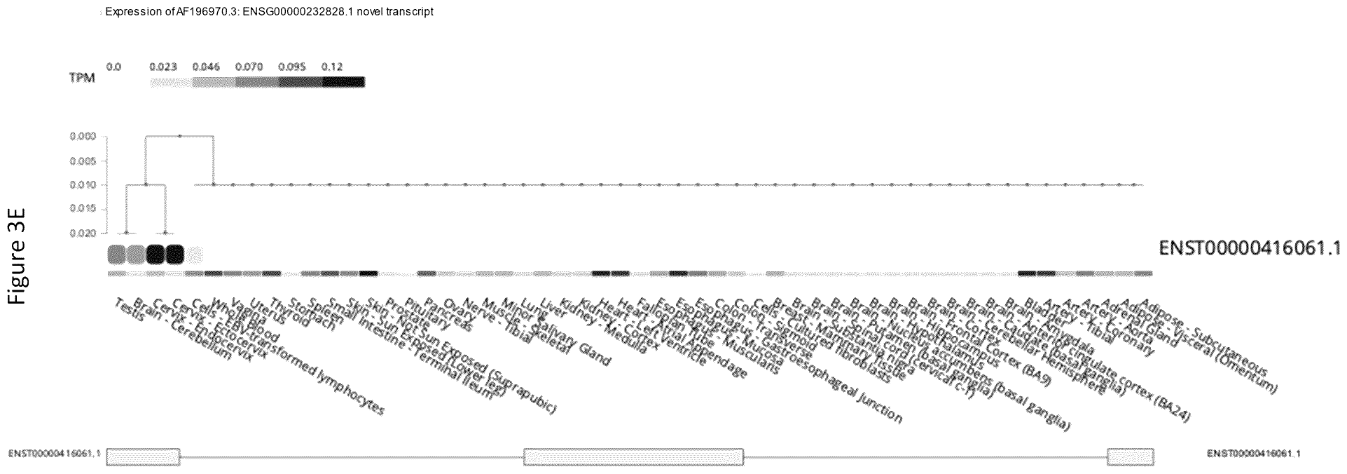

AF196970.3 is expressed in many different healthy tissues, with higher expression in cervix, brain, ovary, and uterus (FIG. 3A), similar in part to SUV39H1 expression (FIG. 3B). It is also detected in many different cell types, with highest levels in endothelial cells, fibroblasts and myocytes (FIG. 3B), similar in part to SUV39H1 (FIG. 3D). Only one AF196970.3 transcript (ENST00000416061.1) was found to be expressed in the GTEx project (FIG. 3C).

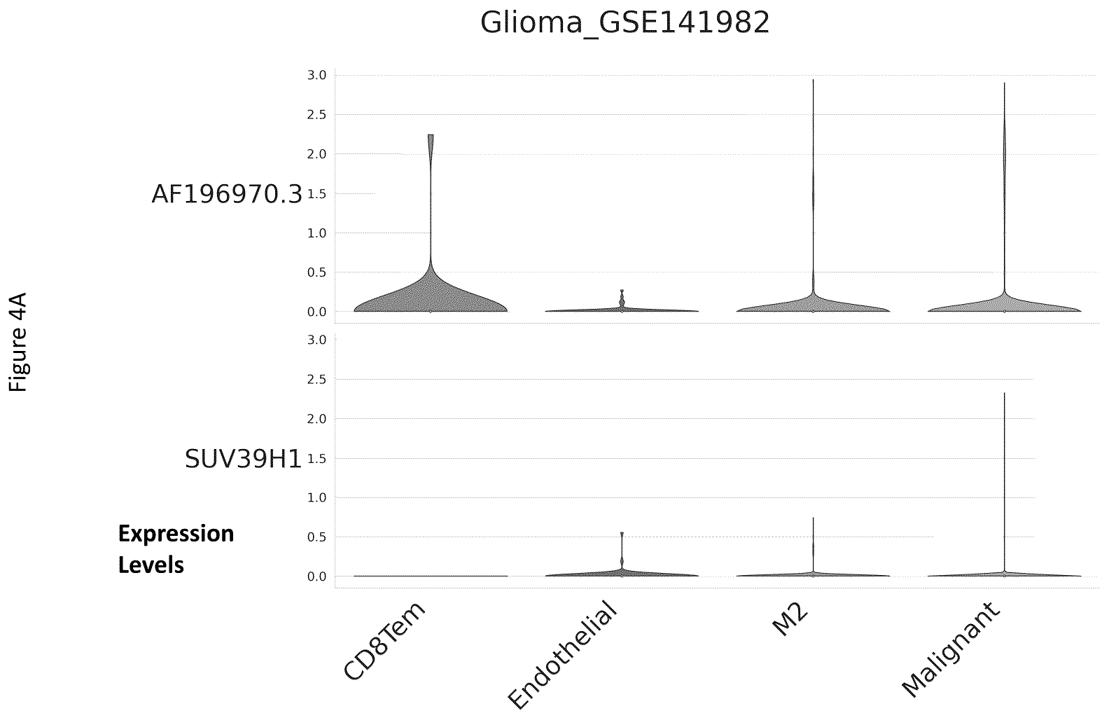

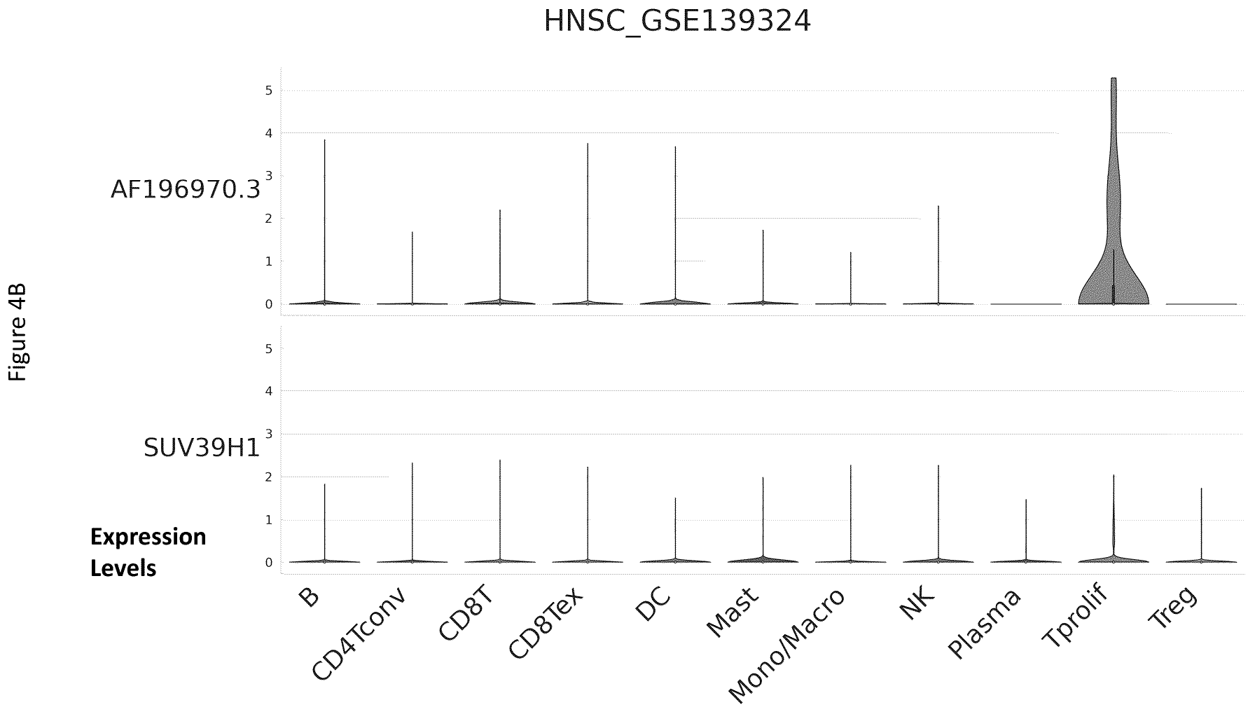

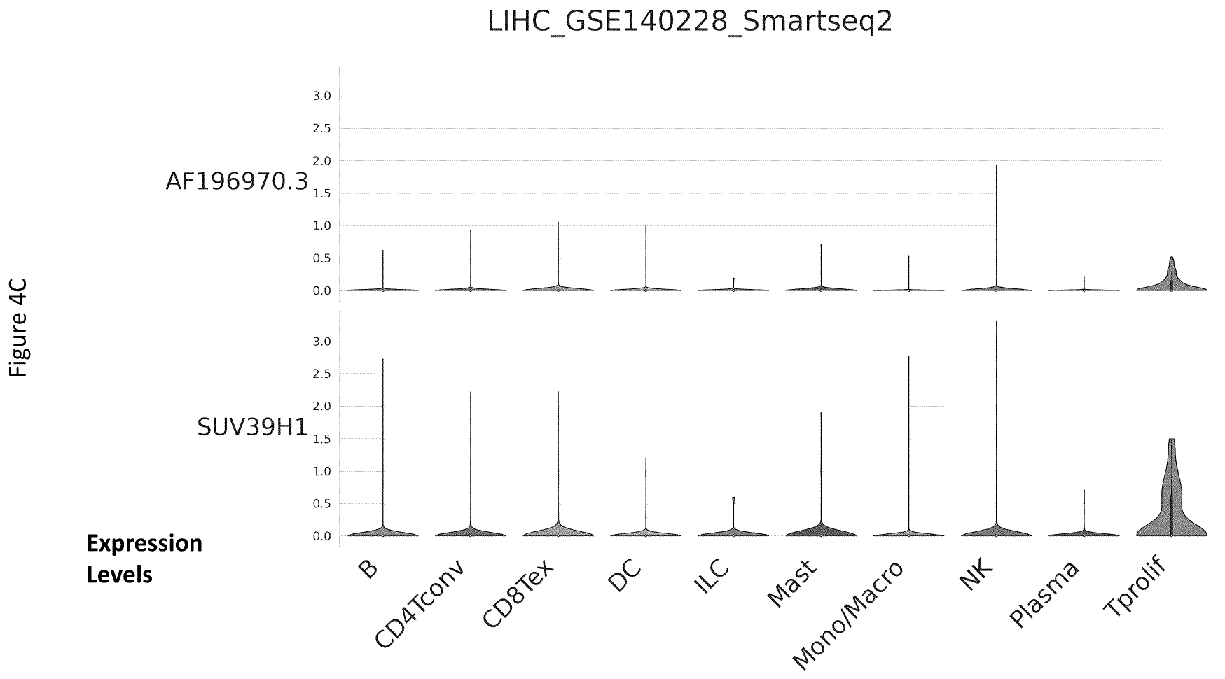

Expression of lncRNA AF196970.3 was examined in human T cells, particularly in the context of cancer. AF196970.3 was found to be expressed in tumor infiltrating lymphocytes in three different projects: a glioma (Wang et al, 2020) (FIG. 4A), a head-and-neck squamous cell carcinoma (Cillo et al, 2020) (FIG. 4B), and a hepatocellular carcinoma (Zhang et al, 2019) (FIG. 4C). AF196970.3 levels of expression correlated with SUV39H1 levels and were higher in proliferating T cells (FIGS. 4B, 4C).

Primers are designed to measure AF196970.3 expression by RT-PCR in human T cells freshly isolated from PBMCs, and T-cells from tumors, both activated in vitro or not activated. Expression patterns were detected.

### Example 2: Overexpression of lncRNA AF196970.3 in HEK293 Cells

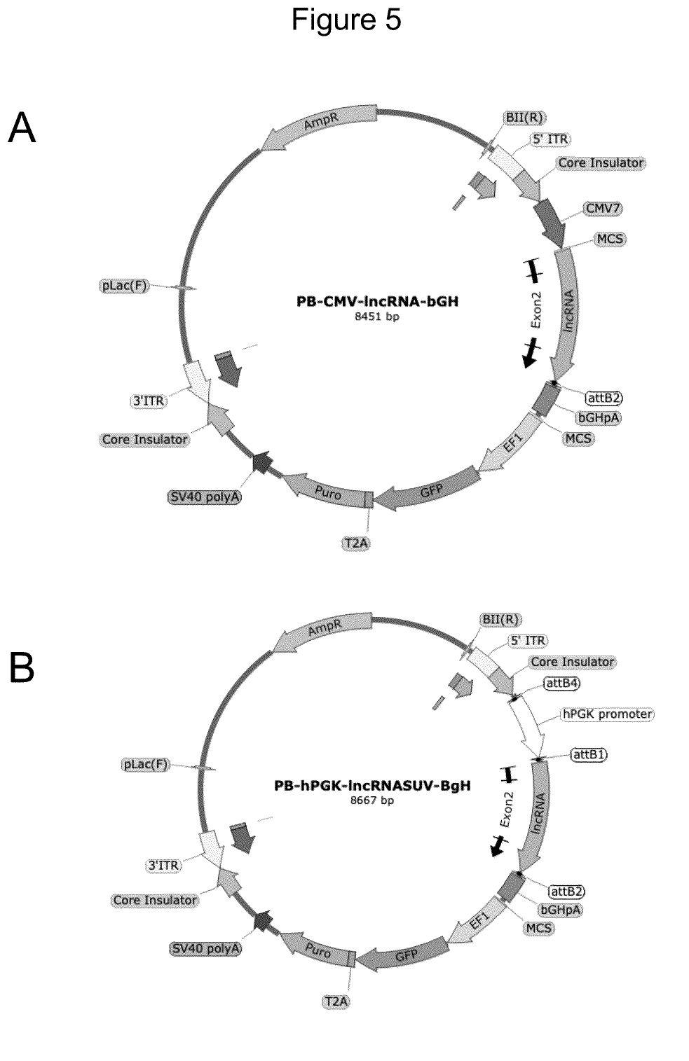

Piggy Bac backbones with GFP-puromycin reporter including lncRNA SUV39H1 exonic sequence with a CMV promoter or with a hPKG promoter are illustrated in FIG. 5 a-b respectively. lncRNA AF196970.3 has been overexpressed in HEK293 FT cells to evaluate its effect on SUV39H1 expression and activity.

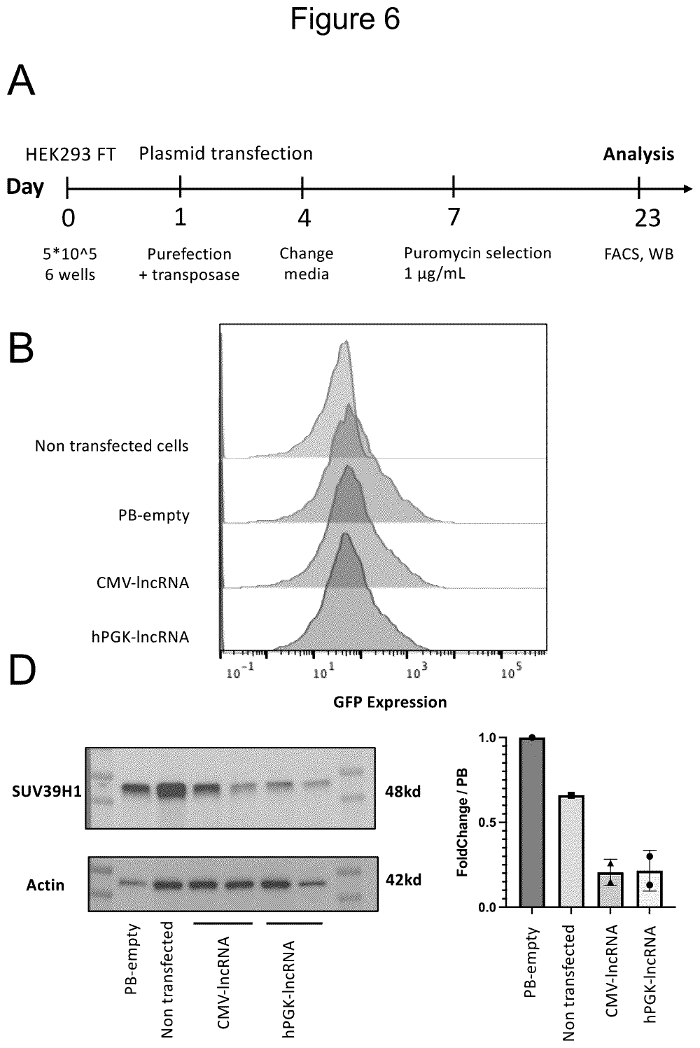

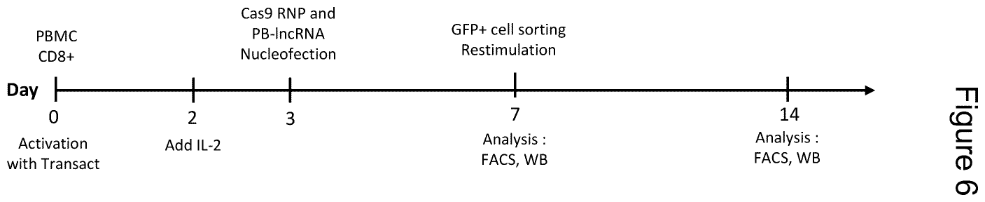

Briefly, HEK293 cells were plated with 5.10⁵ cells on day 0. On day 1, cells were transfected with Piggy Bac (PB) constructs and Super PiggyBac Transposase Expression Vector (use of Purefection transfection reagent). On day 4, media was changed to remove remaining reagents. From day 7, the transfected cells were selected with puromycin 1 μg/mL. On day 23, the cells were harvested, and analyzed for expression of GFP and SUV39H1. FIG. 6b shows GFP expression levels by flow cytometry and comparison of non-transfected cells with cells transfected with either Piggy Bac construct empty or one of the lncRNA SUV39H1 Piggy Bac plasmid as above referred. FIG. 6d shows the quantification of SUV SUV39H1 protein level normalized against actin. The results show that at D23 post transfection, the SUV39H1 protein level is decreased for both lncRNA constructs as compared to the PB empty vector.

### Example 3: Effect of lncRNA AF196970.3 Overexpression on Human T Cells

AF196970.3 is cloned into lncRNA expression vectors, including the PB-CAG-BGHpA (Addgene #92161) (Yin et al, 2015, Cell Stem Cell). Alternative vector systems include the ELECTS transposon system (see Zhang et al., Overexpression of lncRNAs with endogenous lengths and functions using a lncRNA delivery system based on transposon. J Nanobiotechnol. 19, 303 (2021)).

Experimental procedure for lncRNA Suv39h1 (AF196970.3) overexpression in CD8+ T cells is described in FIG. 6d. Briefly, T cell activation is performed on day 0. On day 2 addition of IL-2. On day 3, cells are nucleofected with Piggy Bac (PB) constructs and Super PiggyBac Transposase Expression Vector. Cas9 ribonucleoparticle (RNP) nucleofection are performed to obtain SUV39H1-KO cells as control. On day 7, the GFP positive cells are sorted and then analysed for expression of SUV39H1 (Western Blot), and the memory marker CD27, as well as the tri-methylation of H3K9 (FACS). Remaining cells are restimulated with transact for new analysis at day 14

In another approach, AF196970.3 is upregulated in a cell using Gene-Activating CRISPR as described in: Gene-Activating CRISPR (Rankin et al., Overexpressing Long Noncoding RNAs Using Gene-activating CRISPR. J. Vis. Exp. (145), e59233 (2019).

These approaches are used to overexpress AF196970.3 or portions thereof in human T cells. Levels of AF196970.3 are quantified, e.g by RT-PCR. The effect of AF196970.3 overexpression on 1) SUV39H1 protein levels (by Western Blot), 2) H3K9me3 levels (by FACS), 3) T cell phenotype (CD27, CCR7, CD62L expression by FACS) is observed and compared to SUV39H1 inhibition by CRISPR Knockout.

AF196970.3 overexpression inhibits SUV39H1 expression, as measured by levels of SUV39H1 protein, levels of SUV39H1 activity, the level of the tri-methylation of H3K9, or T cell phenotype (increased memory phenotype as described in PCT/EP2020/070845. Briefly, to observe the expression of central memory T cell surface markers important for the memory phenotype of CD8+ T cells, the T cells overexpressing AF196970.3 can be stimulated with aCD3+aCD28 beads for one week and then analyzed by flow cytometry. The central memory T cell markers CCR7, CD27 and CD62L typically show increased levels of expression when Suv39h1 is silenced in T cells. Additionally, the fraction of CCR7+CD45RO+CD27+CD62L+ cells, which constitute the central memory cell subset, is also typically increased when Suv39h1 is silenced in T cells. Typically, Suv39h1 silencing increases the fraction of Central Memory Cells.

The effect of AF196970.3 overexpression on human CAR T cell efficacy in a xenogeneic mouse model can also be compared to SUV39H1 inhibition by CRISPR KO.

### Example 4: Effect of lncRNA AF196970.3 Inhibition on Human T Cells

Expression of AF196970.3 is inhibited to study its effect on human T cells. The gene is inactivated by CRISPR-Cas9 using gRNAs designed to target the first exon (SEQ ID NO: 2) or the promoter of AF196970.3.

Levels of AF196970.3 RNA (by RT-PCR) and SUV39H1 protein (by Western Blot) are quantified. The effect of AF196970.3 inhibition on 1) H3K9me3 levels (by FACS), and 2) T cell phenotype (CD27, CCR7, CD62L expression by FACS) is observed.

Inhibition of AF196970.3 is expected to increase levels of hSUV39H1.

### Example 5: Design of shRNAs Silencing Suv39h1 Gene and Overexpression in Human Cells

Disruption of the SUV39H1 histone methyltransferase leads to an enhanced T cell memory phenotype and persistence in vivo due to lack of repression of stemness and memory-associated genes. The knockout can be used in the context of adoptive T cell therapy and lead to long-term enhancement of CAR T cell activity resulting in increased survival in preclinical models. Based on this knowledge and on the results obtained with lncRNA as shown in the previous examples, short hairpin RNAs (shRNAs) have been designed to silence Suv39h1 gene expression and inhibit or reduce SUV39H1 protein level and/or activity.

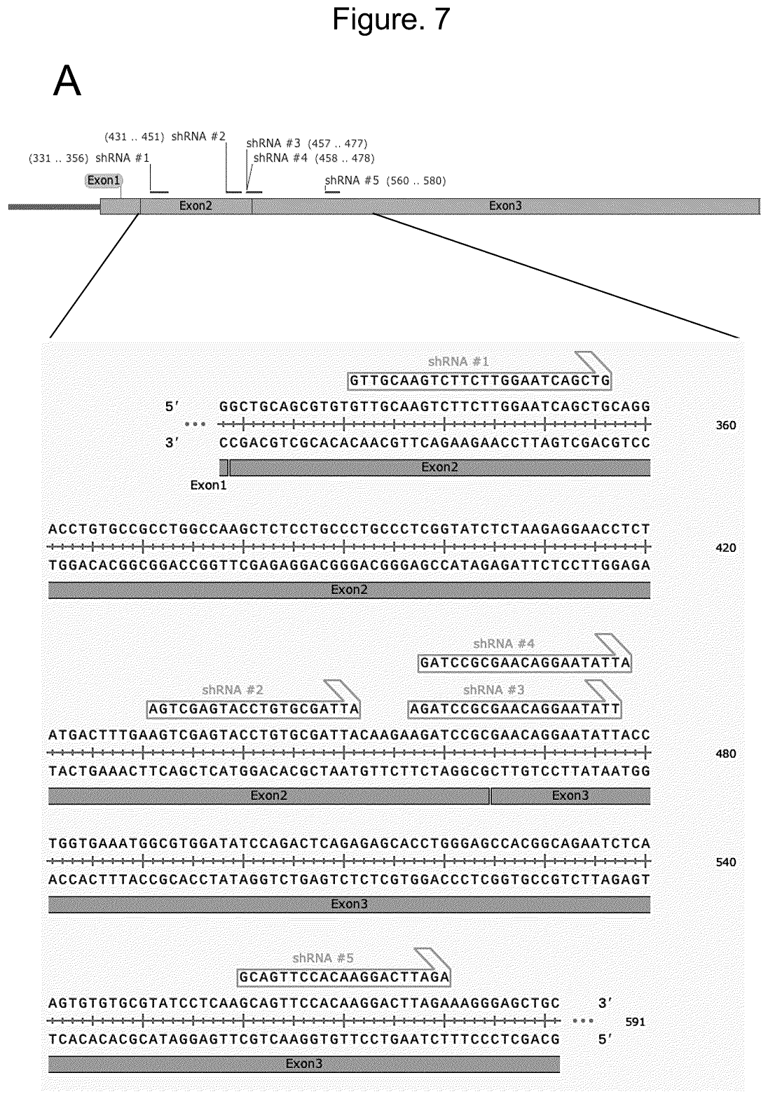

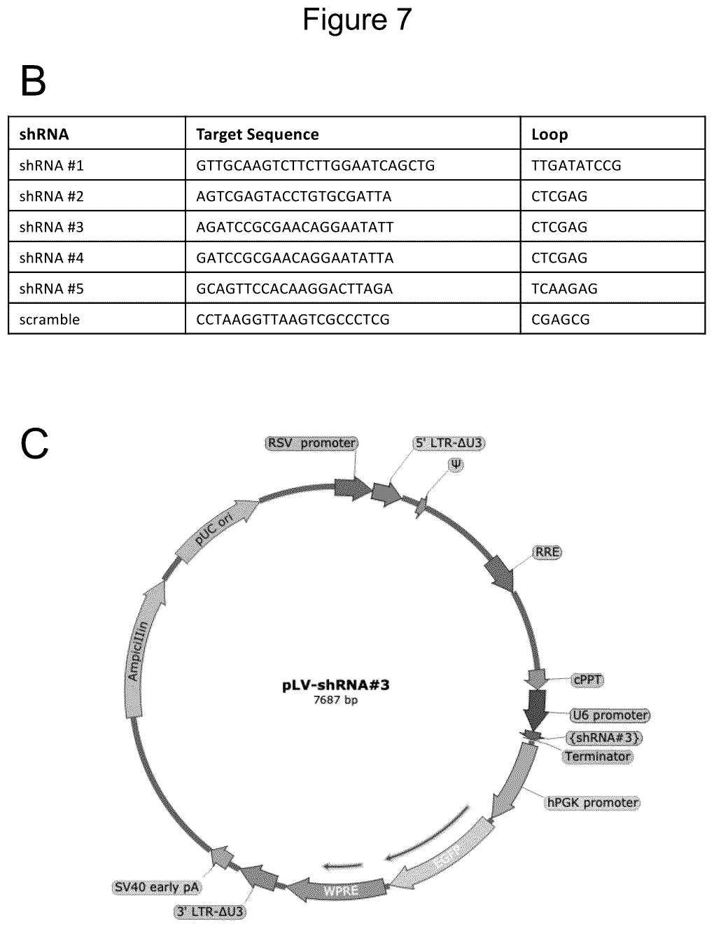

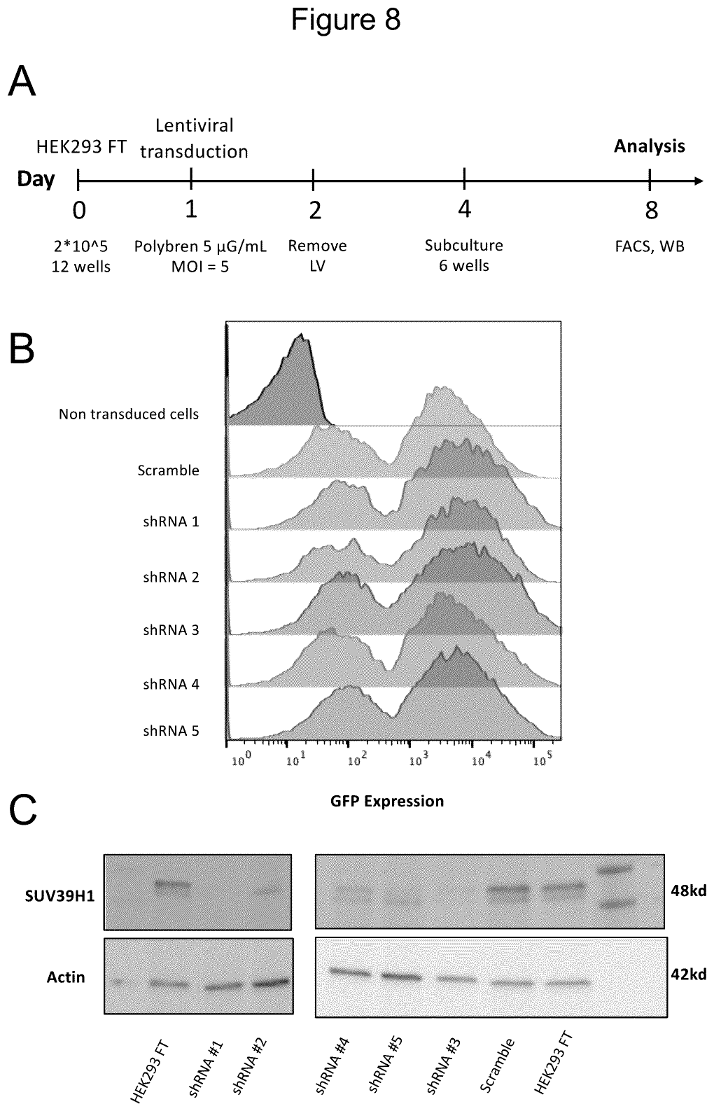

FIG. 7a shows representation of shRNA target sequences on SUV39H1 gene. FIG. 7b shows the target and loop sequences of different shRNA (1-5) targeting SUV39H1. FIG. 7c shows the plasmid map of lentiviral construct used for the shRNA expression in cells using a U6 promoter and an EGFP reporter. The experimental proceeding is illustrated in FIG. 8a. Briefly, HEK293 cells were plated with 2.10⁵ cells on day 0. On day 1, cells were transduced with lentiviral particles at MOI=5 with polybren (5 μg/mL), removed after 24 hours. On day 4, cells were splited. On day 8, the cells were harvested, and analysed for expression of GFP and SUV39H1. Comparison of the GFP expression levels, by flow cytometry, in non-transduced cells with cells transduced with either scramble shRNA or one of the 5 shRNA against SUV39H1 is illustrated in FIG. 8b. As illustrated in FIG. 8c, western Blots performed at day 8 reveal a significant decrease of SUV39H1 staining compared to actin staining for shRNA transduced cells compared to HEK293 control, and scrambled shRNA transduced cells, thus underlying a significant Suv39H1 protein level when overexpressing one the shRNA (1-5) as herein disclosed.

### Example 6: Effect of Suv39h1 shRNA Overexpression on Human T Cells

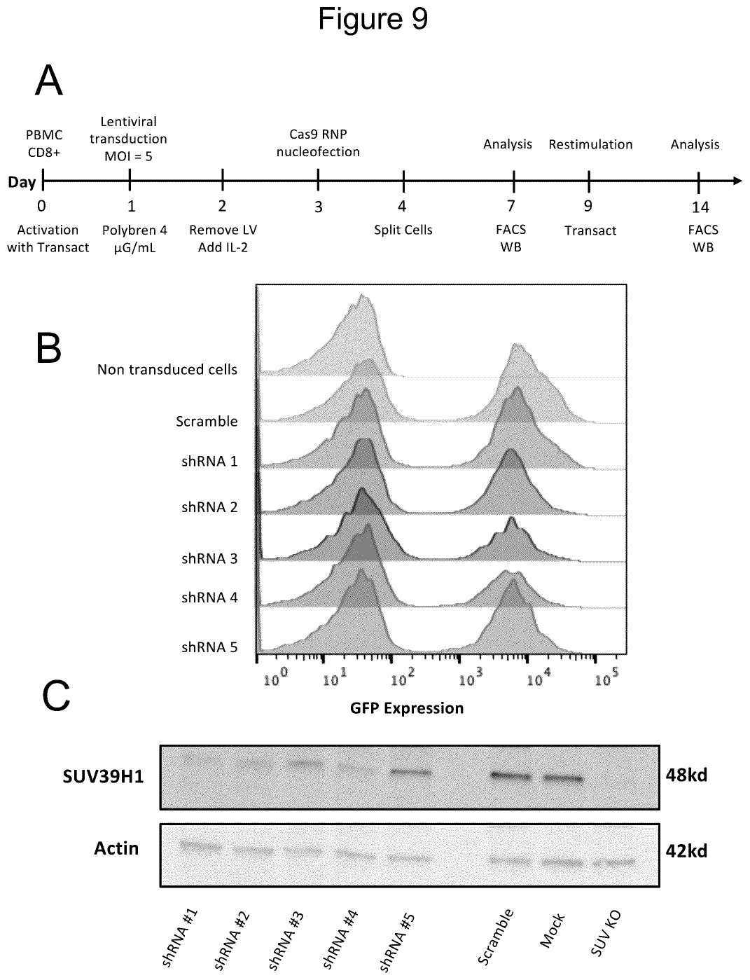

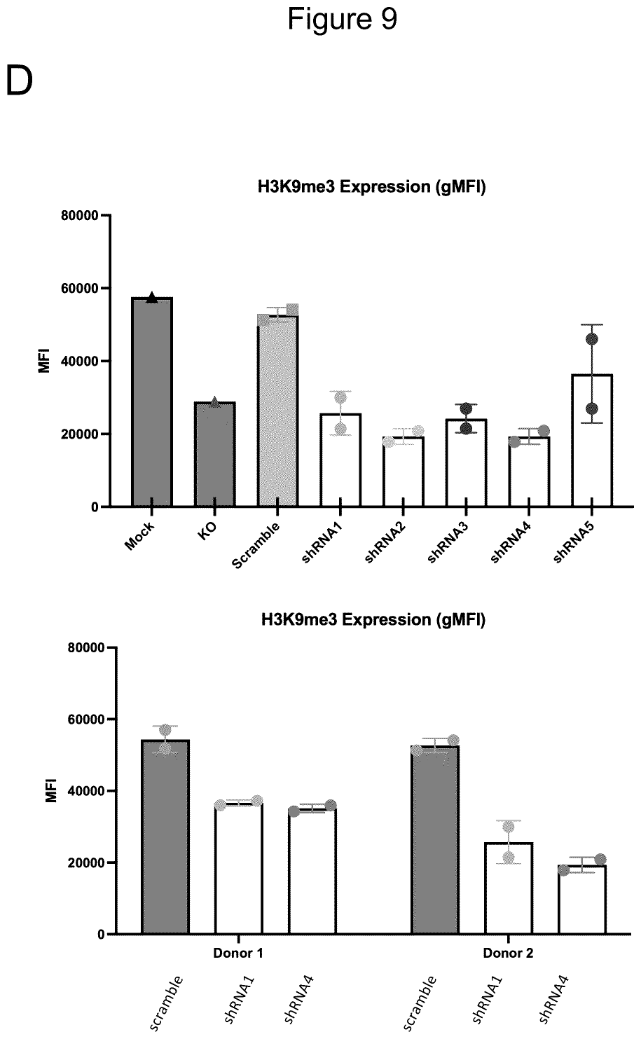

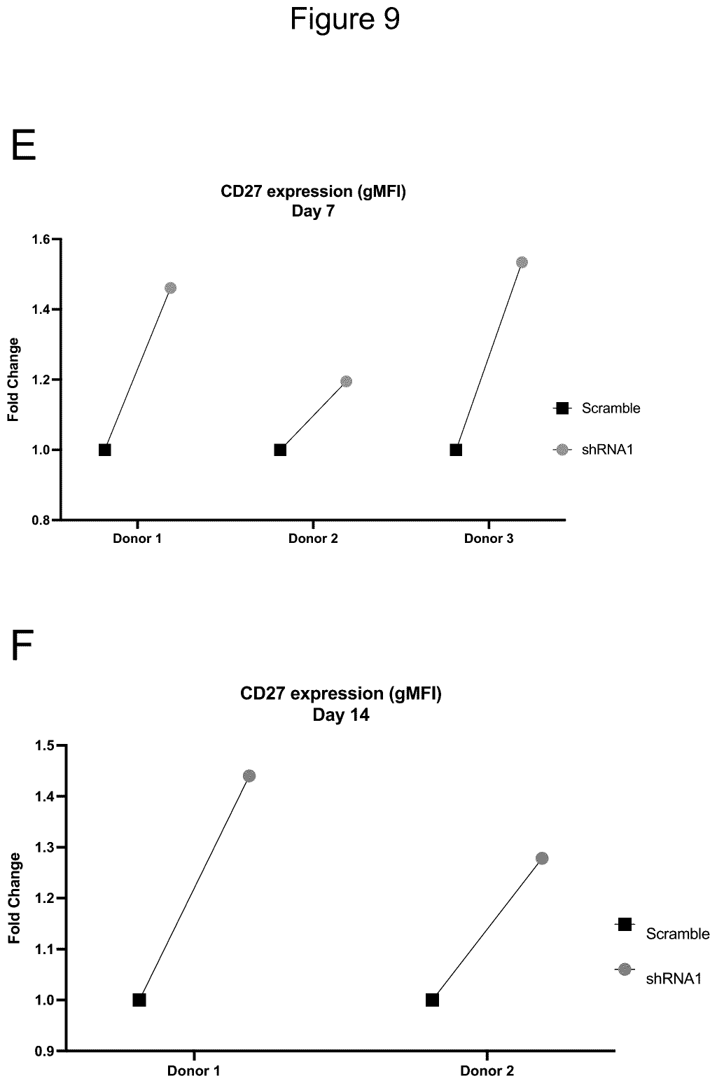

Experimental procedure for shRNA (inhibiting Suv39h1 as above described) overexpression in CD8+ T cells is described in FIG. 9a. Briefly, T cell activation was performed on day 0. On day 1, cells were transduced with lentiviral particles at MOI=5 with polybren (4 μg/mL), removed after 24 hours and addition of IL-2. Cas9 ribonucleoparticle (RNP) nucleofection was performed on day 3 to obtain SUV39H1-KO cells as control. On day 7, the cells were harvested, and then analyzed for expression of SUV39H1, and the memory marker CD27, as well as the tri-methylation of H3K9.

Comparison of the GFP expression levels, by flow cytometry, in non-transduced cells with cells transduced with either scramble shRNA or one of the 5 shRNA against SUV39H1 is illustrated in FIG. 9b. As illustrated in FIG. 9c, western Blots performed at day 7 reveal a significant decrease of SUV39H1 staining compared to actin staining for shRNA transduced cells compared to HEK293 control, and scrambled shRNA transduced cells, thus underlying a significant Suv39H1 protein level when overexpressing one the shRNA (1-5) as herein disclosed. Consistently, FIG. 9d illustrates that in cells transduced with any one of the 5shRNA, the level of H3K9 tri-methylation was significantly decreased as compared to cells transduced with the scramble shRNA and to a similar extend to the level obtained in Suv39h1 KO cells. FIG. 9E-f further illustrate that cells transduced with shRNA exhibit an increased memory phenotype as shown by the increase expression of CD27 at D7 and D14, as compared to cells transduced with the scramble shRNA.

All patents, patent applications, and publications are herein incorporated by reference in their entirety for all purposes and to the same extent as if each individual publication was specifically and individually indicated to be incorporated by reference in its entirety for any and all purposes.

While the foregoing articles and methods of this disclosure have been described in terms of preferred embodiments, and optional features, it will be apparent to those skilled in the art that variations or combinations may be applied without departing from the spirit and scope of the disclosure. Such variations and combinations are intended to be within the meaning and range of the disclosure as defined by the claims. The breadth and scope of the present disclosure should not be limited by any of the above-described exemplary aspects but should be defined only in accordance with the following claims and their equivalents. The terms and expressions which have been employed are used as terms of description and not of limitation, and there is no intention that in the use of such terms and expressions of excluding any equivalents of the features shown and described or portions thereof.

### REFERENCES

Aagaard et al., Functional mammalian homologues of the Drosophila PEV-modifier Su (var) 3-9 encode centromere-associated proteins which complex with the heterochromatin component M31. EMBO J. 1999 Apr. 1; 18(7): 1923-38. doi: 10.1093/emboj/18.7.1923. PMID: 10202156; PMCID: PMC1171278.
Ahmad et al., Long Non-Coding RNAs and Their Potential Roles in the Vector-Host-Pathogen Triad. Life (Basel). 2021 Jan. 14; 11(1): 56. doi: 10.3390/life11010056. PMID: 33466803; PMCID: PMC7830631.
Allan et al., An epigenetic silencing pathway controlling T helper 2 cell lineage commitment. Nature. 2012 Jul. 12; 487(7406): 249-53. doi: 10.1038/nature11173. PMID: 22763435.
Cillo et al., Immune Landscape of Viral- and Carcinogen-Driven Head and Neck Cancer. Immunity. 2020 Jan. 14; 52(1): 183-199.c9. doi: 10.1016/j.immuni.2019.11.014. Epub 2020 Jan. 7. PMID: 31924475; PMCID: PMC7201194.
Nicetto et al., Role of H3K9me3 heterochromatin in cell identity establishment and maintenance. Curr Opin Genet Dev. 2019 April; 55:1-10. doi: 10.1016/j.gde.2019.04.013. Epub 2019 May 16. PMID: 31103921; PMCID: PMC6759373.
Pace et al., The epigenetic control of stemness in CD8+ T cell fate commitment. Science. 2018 Jan. 12; 359(6372): 177-186. doi: 10.1126/science.aah6499. PMID: 29326266.
Rao et al., A drive in SUVs: From development to disease. Epigenetics. 2017 Mar. 4; 12(3): 177-186. doi: 10.1080/15592294.2017.1281502. Epub 2017 Jan. 20. PMID: 28106510; PMCID: PMC5406210.
Wang et al., The Phenotypes of Proliferating Glioblastoma Cells Reside on a Single Axis of Variation. Cancer Discov. 2019 December; 9(12): 1708-1719. doi: 10.1158/2159-8290.CD-19-0329. Epub 2019 Sep. 25. PMID: 31554641; PMCID: PMC7161589.
Yin et al., Opposing Roles for the lncRNA Haunt and Its Genomic Locus in Regulating HOXA Gene Activation during Embryonic Stem Cell Differentiation. Cell Stem Cell. 2015 May 7; 16(5): 504-16. doi: 10.1016/j.stem.2015.03.007. Epub 2015 Apr. 16. PMID: 25891907.
Zhang et al., Landscape and Dynamics of Single Immune Cells in Hepatocellular Carcinoma. Cell. 2019 Oct. 31; 179(4): 829-845.c20. doi: 10.1016/j.cell.2019.10.003. PMID: 31675496.

Sequences

SEQ ID NO: 1
lncRNA AF196970.3 RNA sequence
CGAGGCAGGGCUUUGGCUACUGGAGAUCGUAGGUUCGAAUCCCGUCUGGGAAGUUCAACUUGUG
CACCUGUAAAAGAAGCUGGCAUUAUUGGCUUGUACUCAAGGGCUGGCACAGAGUGUGUCGGGGU
GCGGACGCCCCAGCCACGCCCAUCAUCCGUGCGGAAGAUGCAGAGGUCAUAUCGGAUACCCUUCUG
UACCACACGAUUUGGGCAGUCAUAGCCGCAGCGGCAGCGGGAGUUGCACUCGUAGAUGGGCAGCC
CGGCUCGAAGCCGCACCUGGCCCUGGUCAUUGUAGGCAAACUUGUGCAGUGACGCCCCCGGGCAGC
AGCCUCCAGUGGGUGCCCACAGACAGUCCUGGCACUCGCAGCCCACAGCCACCUGGUUGAGGGUGA
UGCCCUCACCAACACGGUACUCAUUGAUGUACACGAAGGCCCGCGGAGGGCCGUCCAGGUCCACCU
CAUUCUCUACAGUGAUGCGUCCCAGAUGGCUGCGCUUGGCAUUGAGCUCCUGCUCCCAGCGACGG
AGCGCCCGCCUCUGCUUGGCCUUCUGCACCAGGUAGUUGGCCAAGCUUGGGUCCAGGUGCCGGGG
GGUCUUUGACCGGUGGUGCCGCCGGAGCAGCUCCCUUUCUAAGUCCUUGUGGAACUGCUUGAGG
AUACGCACACACUUGAGAUUCUGCCGUGGCUCCCAGGUGCUCUCUGAGUCUGGAUAUCCACGCCA
UUUCACCAGGGUCAAAGGAGAAAAUUCCCUUUGGAAACAGAUGUGGGCAGUUGGGGACAAGAGG
GCAGGACACUAACUUCCUUGUGACCUGUCCCCUCCCAGAGCAUGGUCACCCCAGACUCACGCGGAU
CUUCUUGUAAUCGCACAGGUACUCGACUUCAAAGUCAUAGAGGUUCCUCUUAGAGAUACCGAGGG
CAGGGCAGGAGAGC
SEQ ID NO: 2
lncRNA AF196970.3 exon 1 RNA sequence
CGAGGCAGGGCUUUGGCUACUGGAGAUCGUAGGUUCGAAUCCCGUCUGGGAAGUUCAACUUGUG
CACCUGUAAAAGAAGCUGGCAUUAUUGGCUUGUACUCAAGGGCUGGCACAGAGUGUGUCGG
SEQ ID NO: 3
lncRNA AF196970.3 exon 2 RNA sequence
GGUGCGGACGCCCCAGCCACGCCCAUCAUCCGUGCGGAAGAUGCAGAGGUCAUAUCGGAUACCCUU
CUGUACCACACGAUUUGGGCAGUCAUAGCCGCAGCGGCAGCGGGAGUUGCACUCGUAGAUGGGCA
GCCCGGCUCGAAGCCGCACCUGGCCCUGGUCAUUGUAGGCAAACUUGUGCAGUGACGCCCCCGGGC
AGCAGCCUCCAGUGGGUGCCCACAGACAGUCCUGGCACUCGCAGCCCACAGCCACCUGGUUGAGGG
UGAUGCCCUCACCAACACGGUACUCAUUGAUGUACACGAAGGCCCGCGGAGGGCCGUCCAGGUCCA
CCUCAUUCUCUACAGUGAUGCGUCCCAGAUGGCUGCGCUUGGCAUUGAGCUCCUGCUCCCAGCGA
CGGAGCGCCCGCCUCUGCUUGGCCUUCUGCACCAGGUAGUUGGCCAAGCUUGGGUCCAGGUGCCG
GGGGGUCUUUGACCGGUGGUGCCGCCGGAGCAGCUCCCUUUCUAAGUCCUUGUGGAACUGCUUG
AGGAUACGCACACACUUGAGAUUCUGCCGUGGCUCCCAGGUGCUCUCUGAGUCUGGAUAUCCACG
CCAUUUCACCAG
SEQ ID NO: 4
lncRNA AF196970.3 exon 3 RNA sequence
GGUCAAAGGAGAAAAUUCCCUUUGGAAACAGAUGUGGGCAGUUGGGGACAAGAGGGCAGGACAC
UAACUUCCUUGUGACCUGUCCCCUCCCAGAGCAUGGUCACCCCAGACUCACGCGGAUCUUCUUGUA
AUCGCACAGGUACUCGACUUCAAAGUCAUAGAGGUUCCUCUUAGAGAUACCGAGGGCAGGGCAGG
AGAGC
SEQ ID NO: 5
Genomic DNA sequence
TGCTGTTTCGGCGCAAGCGCTTTGGCTCCTGGGTTGGCGTTCAGGGTTGCCGAGGCAGGGCTTTGGCTA
CTGGAGATCGTAGGTTCGAATCCCGTCTGGGAAGTTCAACTTGTGCACCTGTAAAAGAAGCTGGCATTA
TTGGCTTGTACTCAAGGGCTGGCACAGAGTGTGTCGGGTAATCTTTTCCATTTTCTTTTACCTATTCTTTCT
TTTTTTAAGCAATGCCTATTGCATCCCATTATATTGATTTCAGGACCCACTCACCGTTCACAACAGCACTA
AGAAAAATTCTTCAGGGTCCCTGGCATTTGTCTTTCAGGCTACCTGTAGGTCTGAGTCCAGCAGATAAGA
TTCAAGGTGATCTCATGATCCTCCTCCAGGCCAGCCCCCATCTCCCAGAAGAGGGTGATTTGGAGGCTTC
CCTCAGGACCAGGCCTGACTAGGAGTGTGATGGGGTGGGCTATAAGTGATTAAAAGAGAGATGCATGG
TTTCACGCTCTGGGATGCTGAAGAGCTTGTGGCAACTACAAGGAGAATTTTGGAAGGTGGGGGCAAGA
CTTAAAAGAGTACGCTTTTAAATGATGGGATTTTAAACATCAGACAGGGGAGGTGTTTGGCTGGTGAGG
TAGGTAGGTAGTAGGAAGAAAGTGATTCTCAAGTCAAGTCAGCTCATCATAGGGTAGTTATTACACTCT
GCAGTTTGGAGGACTGTGTGCAGGATGTGAAGTCTGATTTGTTTTGGAATCTCAGTTCAGGGTATGACT
TATAAAAAGACTGTGAAGTCTCTTTGCTGTTTGAAAATCTGTGTCTCAGAGGTTTAGGTGTTTGAGATCT
TTGTGGGTCTCTGTCCCAGGGTTGGTTTCTTTTTTCTTTCTTTTTTTTTTGTTTTTTTTTGTTTTTCTTTTCTGT
TTTTCTGTTTACGTTGGTCCAGAATGTCCCAAGGTTTCTAAGCTGTTTATGGATCTTTGTCCCCGGATGTG
CAGAGTTTTTTTTTCTTTTCTTTTTTTGTGTGTCTTTACTTTTATTTATTTATTTTTGAGACAGGATCTTGCTC
TGTCACCCAGGCTGGAGTGCAGTGGTGTGATCTCGGCTCACTGCAGCCTCAACTTGCCAATCTCAGGTG
ATTCTCTCACCTCAGCCTCCTGAGTAGCTGGGACTACAGACACGTGCCACCACACCCGGCTAACTTTCTTT
ATTTTTTGTGGAGGTGGGATTTTACTATGTTGCCCAGGCCTTTTTGTATGTTTTTAGGGGCAGGGTCTCTC
TCTGTTGCCCAGGCTGGAGTGCAGTGGTGCAATCATGGCTCACTTCAGCCTCGACCTCTGGCTCAAACAA
TCCTCCCACCTTAGCCTCCTGAGTAGCTGGGACCACAGGTGCGTACCACCACACTTGGCCCGGCTATTTT
TTTTTTTTCTTGTAGGCTGGGCACGGTGGCTCACACCTGTAATCCCAGCACTTTGGGAGGCCAAAGTGGG
TGGATCCCTTGAGCCCAGGAGTTCGAGACCAGCCTGGCCAACATGGTGAAACCCTATCTCTACAAAAAA
AAAAAAATTAGCCGGGTATGGTGACACGTGTCTGTTGTCCCCAGCTACTCAGGAGGTCAAGGCTGCAGT
GAACTGTGATCTCACCACAGCACTCCAGCCTGGGCAACAGAGCAAGACCCTGTCCCAAAATAATTTTTTT
TGCGTGTGGAGACAGGGTCTCCCTCTGTTTCCTAGGCTGGTCTCAAACTCCTGGGCTCAAATGATACTCC
TGACTCAGCCTCTCAAAGTGTTGGGATTACAGATGTGAGCCACCACACTCAGCCTTTTTTTTTTTTTCTTA
AAATTGATTTTCATTTTTCTTTTATTGGCATTTCTGTGACTGCTAGAAGGGCTTAACATTGTTTTCTGTGCT
TGTTGGCAAGATGTATCTAAGTTTTGTTTTGTTTTGTTTTGAGACAGGGTCTCACTATGTTGCCCAGGCTG
AGTACAGTGGCATGCACATGGCTCACTGCAGCCTCGACCTCCTGGGCTCAAGCAGTCCTCCCACCTCAGC
CTCCTGATCTTGAGTAGCTGAGACCACAGGCATGTGCCACCACACTCCACTAATTTTTTTATTTTTTGTAA
AGACCACGTCTCACCATGTTGCCCAGGCTGGTCACGAACTCCTGGACTCCTGTCTTACCCTCCCAAAGTG
CTGGGATTACAGGCATGAGCCACTGCACTTGGCCTTATTTAAGTTTTCTAAATTCCCTTTTACGTCCTGTG
ATCATTTTTGTTAAAATTTTTATTTTATTTTATTTTTTGGACACAGGGTCTTGCTTTGTCACCCAGGCTGGA
GTGTACTGGCGTGATCATGGCTCACTGCAGCATCGACCTCCTGGGCTCAAGCAATCCTCCCACCTCAGCC
TCCCAAGTAGCTGGGAATACAGGTGTGTACCACCACCCTTGGCTAATTTTTGTATTTTTGTAGAGAAGGG
GTTTCACCATATTGCCCAGGCCGGTCTCCAACTTCTGAGCTCAAGCGATCTACCCACCTCAGCCTCTGAAA
GTGCTGGGATTACAGATGTGAGCCACTGCGTCCAGCCTTTAACATTTTTTAAATTTTAAAAGCATTTTAGA
CTTAAAAAAATTATAAAAATAGTGCAGAGAGTTCTTATATACTCTTTTTCTCCTAATGCTTACTTATATAAT
CTTAGTACAATTTTTAAAATCAGGAAATGAATGACACGGTTATTTATGAGCTGTTGTTCACCGGTTTGTGT
CCAGGGTGTGAAATATCTGAGCTCTTTGGACCCAGAGCTCTCTTGGGTTTTGTGTCTCATGATGTGATGT
CTCTGAACATTTGGTATATGTTTTTAGATGCTAGGTCTTTCGTCATCTTGGTAGTCTTTGTCCCAGAGTAT
GATGTCTTTGACCTGTTAGTAGTCTCTTTAGGCTTGTCTTCTCTGGGCTGTATGGGCTTCCATGTTCAAGG
TGTGAGGTTTCTGAGCACTTTGAAGGACTATGTTCAAAGATGTGTATACTGTCAGCTGGTTTTTATTGTCT
TCTATCAGAGATAATGAGGTCAATAAAAACTTTTCGGTTCTGTATCCAGGGCAGGGTTCCTCAGCCTTGG
GACTGTTGACATTTTGGACCAGAAAATTCTTTGTTATGGAGGGCTGTTCTGTGCATTGTAGGGTCTATTG
TATTTGTTTACTAGGGCTGCTGTCATGACAAAGTACCACAATCTGGGTAACTTAAATAGCAGAAATGTAT
TGTCTCACAATTCTGGAGGCTGGAAGTGTGAGATCAAGATGTTGGCAGGGTTGGTTCCTTTGGGGCCTA
TGAGGGGAATGTGTGCCCTGCATCTTTCCTAGCTTCTCATGCCTGCTGGCAATCTTTGCTGTTCCCTGGTG
TCTGCTGCATCACCCCATCTCTGCTTTCATCTTTACCTTGCATTCTCCCTGTGTGTCTTGTGTCCAAATTTCC
GCTTTTTATAAAGACACCAGTGGCGCAGGATTAGGGGCCCACTCTACTCCAGCATGATCTCTAACTTAAC
TAATTACATCTGCAACAACCCTATTTCCAAATAAGGTCACATTCTGAGGTACTGGGGTTTAGGACTTCAA
CGTAGGAATTTTGAGGGGACATAATTTCACCCATAAGAGACGTTTAGCATGAGAGAAAAAGGGAGGGA
AGAAGGGAGCAGCCTCTGACACCCAGAAGCAGGCCCGGCCCCCATAGGTAGGCCATGTTATTCTCCTAA
CGGACGTGAACAATCTCACAGAATACCAGCGTCAGGTAAGGTTAGTCTGAGACCATGATAAAATCGACA
GAGCTTGGCCGGATGAGGTGGCTCACGCCTGTAATACTAGCACTTTGGGGGGTTGAGGTGGGCCGATC
GCTTGAGCTCAGGAGTTCGAGACCAGCCTGGACAACATGGTGAAACCCCGTCTCTACAAAAAATACAAA
AATTAGTCGGGCATGGTGGTGGGTGCCTGTAATCCCAGCTACTTGAGGGACTGAGGCCAGAGGATTGC
TTGAACCCAGGCATTTGAGGTTGCAGTGAGCCGAGATCATGCCACACTGCACTACAGCCTGGGGATAAA
GTGAGAACTTGTCTCAAAAAAATAATAAAATAGGCCGGGTGCAGTGGCTTACGCCTGTAATCCCAGCAC
TTTGGCAGGCCGAGGCGGGCAGATCACGAGGTCAGGAGATCGAGACCATCCTGGCTAACACGGTGAAA
CCCCGCCTCTACTAAAAATACAAAAAATTAGCCGAGCGTGGTAGCGGGCACCTGTAGTCCCAGCTACTC
GGGAGGCTGAGGCAGGAGAATGGCGTGAACCCGGGAGGCAGAGCTTGCAGTGAGCCAAGATCGCGCC
ACTGCACTCCAGCCTGGGTGACGGAGCGAGACTGTGTCTCAAAATAAAAATAATAATAATAATAATAAT
AATAAAATAATAAAATAGAGCACAGCCACTTTATAATGTTGTTTAAGCACAAACAAAAATAAGGATTCAC
TGTGTTGCCCATAAAATACTAAACATTCTCCTCTCTTGGTTAAAATGAGTGACTGCCACTTCCTTACCAAT
GACAGCGTTAGCCTCACTCTAGTCTGCCCTCCCTATAGATAAGATTTATTGAAATACCCAATTGTAAAATT
GCCCTTGTTTTCTGACAGCATCTAATCTAGAGTGAGCCCCCACTTCCTTAGACCCTACCCCCAAATCACTC
AATCAAACCCGACCACCCCACACTTCCCCATGGAGTGCGTTCTCCCTCACTGCAATGAGTAATCATCCCA
ACTTGTTCCACTACAGGTATGTTCCTGGGTGGGGCGGGGGTCTTTGGCTAAAGGACACTGGCAAGCTGT
ATCCCTGGCTTCAACTCACAGATGACAGTAGCACGCCCGCAACACATATTTGTGACAGCCAAAATGTCCT
TAGATATTGCCAAATGTTCTTTGCGGGGCAGAATCGCCTCTGGTTGAGAACCACTGGTCTGTGGGGTAT
GAAGCGTTTGAACAATTTAGGGGTCTTTGTCTGAGGTTTATGTTCTCTGAGAGGTGTTTGGGTCTGTGTC
TCAGAGTGTGAGGTCTGTGGTCTGTATTACGGTTTCTGTTCCAAGGTTTGAAAGCTCTGAGCTCTTTCGG
GGTGCTATATCAGGAATTAGTGACCTTTTCACATACCAAATCCAGACATGAGGTATGTGTGACATCTCCT
TTTCCCTCTGCCTGGACCATATGCCAGTTCAGAAAGGCAGTGAGGGTTGGGTGTGGTGGCTCACGCCTA
TAATCCCAGCATTTGGGAGGCCAAGGTGGGTGGATCACTTGAGGTCAGGAGTTTGAGACCAGCCTGGC
CAACATGACAAAACCCCGTCTCTACTAAAAATACAAAAATTAGCCGGGTATGGTGGTGCATGCCTGTAAT
CCCAACTACTTGGGAGGCTGAGGTTGCAGTGAGCCGAGATCGCACCACTGCACTCCTGGGCGACAGAC
CAAGACTCTGTCTCCAAAATAAAGAAAAAGAAAAAGAAAGGTGGTGAGAAGTAGGAACCCCACAATTG
GTTGTGAACTCCTCCAGTACACATTAGGATCGGGATTCACAGCAATCCCAGCCTCTTTGTCTAGACCAGC
AGGCTTCCCTGGGCCACATTGGAAGAATAATTGTCTTGGGCCACACATAAAATAGCTAACACTAACGAT
AGCTGATGAGCTAAAAAAAAAAAATCGCCAAAACATCTCATAATGTTTTAAGAAAGTTTACGAATTTGTG
TTGGGCCACATTCAAAGCCGTCCTGGGCTGCATGTGGCCCATGGGCCTTGGGTTGGACAAGCTTGGCCT
AGACCATAAAGTATAAATTACCAACTTCCTTCAGAACCTATCTCGCCCTCTGTTCTGGAGGGCACAGTTTC
CCTTGACACTGTTTGTGGAGATTGTGTTGTAGGGAGATGTTTGTTCTGTGTCTCCTGACATCAGCCACTG
AGTGGGGACCCCCATCTCTCAGGATGGACGTTGCTCAGATGGATGTTGGAGTCCATCTCCCCTTAGGCTT
TGATCACACTGATGATAACCCAGAACACATGGACTTTGGAGCCTGATAGACCCTCTTTCAAAGCCTGGAA
CCCCCAAGTCCTGGTTGGATGGTAGGAGAGTCTAGCGTTTTGGATCATCTAAACCTTGAGCTGCTGAGG
TTTTTACAAGACTGGTAAGAATCGGTAGGACAATGCACAGTCCACACACGCTCTCCTGATTTCAGTCCCT
TCCCCCTAATCCCACACCCCTTCTGAAAGTAGAATTCAGGGCTGGGGGAAGGGAAGCAAGCAATCCATA
TAAATGCCAGGGGCCAGTGTGGGCTTGGGACTGTCTCAGTTGGAACTTAGGAATCATGTGCACATGTTG
TTTAATGTAAATGTATGTTGGGTTTAAGAAAGAGTAAACACTCCCCAGAATTGTGTCTTCATCTCTAGTA
AATCCACAGTTTTACTTCAGTTGTTCTCTTGAGAACATGCATAATAGATTGTGCACCATGAGGCAAGGAT
GCACAGTGAGAAAAAACTTTGTTCCTCTTCTGAAAACTGTGCTTATGAGACGGGTTTATACATTTATGTG
AGAGGCAGGGTATGAGCTCCAGAGAAAGACTTTAGGGGAGGATGCAGGGCCCCAGTAGCTCAAAGTA
GTCTCAAGAGAAATAAAAAATAGTTTGGACTAAATGAAAATCAAAATAAAACTTACCAAATTTGTGGGA
TGCAGCAAAGCAGTACTTACCTAGAGGGAAATTTAAAAGGCACTTATGGCTTCTGCTGTACTTTTTCTTG
GATCACTGACTAGTAGAAGCCAGCCATCATTCGAGGATGCTCAAGCAGCCAAAAGCCGGTAAATTTGGG
GGTTATTTTTTATGTGGCAATAGATAACTCATAAGAGGTGTTTCTGAGCTCTTTGATGGTCTGTCTGGAG
TTCGAGACTTTGGAGCTTTTTCGTGGTCTGTATCCATGCCATGATGTCTGGGTCTTTTGGGCATTCTATGT
TTCGCATTTTTAAGTGACCTTTTGGGGTTCTCTCTCTAGGTTTTTAGATCTCTGAATTATTTGGGAATCTAT
GTCCCAAAGCATGGGGATTCTGGGCTGTTTTTTTCTGTTCATGTTCAGAACGTGATCAAGTCCAGAATTT
GAATTATCTGAGCTCTCTGGCAGTCTGTTCTGGGTATGAGGTCACTGGGGTGTTTGGCAGTCTGTGTCCC
AGGTTCACATCCATGCACTCTTTGGAGGACGGTGTATTGTTTCTGTTTATTCTTTGAGGTATATGGATTTC
TAAGTCCCAGGAAGAGACATCACCAAGGTGACTGGATTCTGCATCCAGAACACTAGGTGTCAGCTGTTT
GGGCCACTGAGTCCCTGGATATGAAGTCTTTAATGTGTTTGAGAACCTGTGTCCCGACTTTGATATTTCT
GAGTTTGAGGGCAGAGGGAATCAGTACTCTGCACTGTTTATGAGTATATATCTCAGGATGTAAGGCTCC
CAAACTGTTTGTGAGTATCCCTGACATTTATGTCACAGTATATGAGATCTCTGTTCATGGGTGTATATACC
AGGGTGTAAGGTATCTGAGCCCTTTGATCGTGTGTGTCCAAAGTTGTGGGGTCTCTAATCTGTTTGAGTC
TATATTTCCCAGAATAAGAGATTTCTGATTTTTTTGTGGGTATATATTCCAAGATGTGAGTTCTGTGATCC
ATTTGGAAGTATATTTGAGGTTTCCGTTCTGTTTGTGGGTATATGTCCAATGGTGTAAGGTTTCCAATGT
GCTTGTGAGTATTCATCCCAGGGTATGAGGCCTCTGATCTGCTTGTCATTATACAATGACAAGATGTGAG
GTCTCTCTTCATGAATGCATGTCAAGGGTTTGAGGTCTCTGATCTGTTTTTGGTTTTATGTCCCGGAATGT
TAGATCTCTGATCTCTTTGTGGGTGTATGTGCCAAGGTGTGAGTCTCAGCTTTTTGTGGCTATATTTCCCT
AGATGTTATATTTCTGAGCTTTTCTGGTTGTATTTTTTTAGGGATGTCAGTACTTTAATCTGTTTATTTGGT
GTGTGTTGGAGAATATGAGGACTATTCTGTCTGAGGGTGTATGTCCTAGGGCATGAGGTCTCTGCTATG
ACTGTGGGTGTACATCTCTGGGTATGAGGTCTCCAAAATGTTTGTGGGTATATTTCCCAGGATGTGAGTT
CCCTGAGCTCCTAGTGGGTGCATGTAAGGTCTCAGATCTGTTTATCCCTGTATATGTTCCTGTATATGTGA
AGTGTCAAGGGTATGAAGTCTCTGATATTTTTGTGGCTATATGTTCCAGGATATGAGGTTTCTAATCTGT
TTGGGGGTGGATTGCTCAGAGTGAGTTCTCAAGTATGTTTGTGGTTATATGAGCTAGGATTTAAGTATCT
GATCTTTTTGGGTGTGTAAGTCCCATGGTGTGAAGTCTCTGAGCTCTTTGATGGTATATATGCAGAGGTG
TGAGATGTCTGATTTGTTTGGTGGTATATTTCCCAGGATGTGAGGTCTCTGATCCATTTGTTGCTGTATAC
TAGTATGTGATTTTCATTGGTGTATGTCAAAGTCAGTTTGTTGGTGTATTTCTCAGGATATGAGGTCTCTG
GTCTGTTTCTGGATGTATGTTCCCAAGTAAGAAGTCTCTGATCCATGTGCCCACATATGTCTCTGTATGTA
AGGTCTCTGATCTGTTCATGGATACACTGAGTTTGAGATCTTTCATCTGGTTGCAGGACAGGCAAGCCCC
AAAGTTGAGGCTTAGCTCAGGAGGGTTCTTGGCTTTGCCTAGGAAGGAATTCAAGAGTGAGCTGGTGG
TGTTAAATAGCAGCTTTTATTGAAGCGGCAGTGTATAGCAGGAGCAGATGTGCTGCTCCTTGCAGAGAA
GAGCTACCCATAGGCAGTATGCCCAAAGTAGCAGCTCAGAGGCCGTTCTGCAGTCATATTTATGCCCAC
CTTTTAGTTACATGCAAATTAAGGGATGGTTTATGCAGAAATTTTAGGTATCTGGGCTATTTCTGCAAGT
ATGTCCCAGGATGTGAGGTTGAGGTCTCTGACATGTTTGTAGATGTATTTACTAGAATAGAAGATCTCTG
ACATGTTTGTGGATGTATGTCCCAGTGTGTGAGGTCACTGATCTGATCTGTGTTTGTGCATGTATGTCCC
AGGATTTTAGGTCTGTGATGTGTTTGTCATTGTAATTATCAGTATGTGAAGCATCTTTCATGGGTATATGC
TAAGAATAGAAGTTCTCTGATATGATTGTGATTGTATGAACCTAGGTATGAGTTCTCTTATCTGTTTGTG
GAGTATGTCCCACAGTGAGAGGTTCCTGAGCTGGTTGGGGGTTTATTTACTAGGATATAAGGCTTATGA
CCTTTTTCTTGTTGTGTGTGAGGTCCGTGGCTATCGTTGTATCATTGTGTATCCTGGTATGTGAGATCTTT
TTCATGGGTGCATGTAAAAAATGTAAGGTCTCTGATCTATTTGTGATTATATGTCCCTGCATATTAGGTCT
CTGATCAGTTTGTGGATTTAATTCCTACAGTTTGGCATCATCAATCTGTTTGCAGGTGTATGTCCCATGAT
GTAATGTCTGATCTTTTCGAGGATGTGTGAGGTATTTCATCTATTCCTCAGTGTGTTTCCAAACATGAGGT
CTCTCAACTGTGTGACAGTGTATGTTCTGTGATTTGAGGCCCCTATTTTTTTTTTACAGGTTTATTCCCAAG
GATATGAACTCTCCTGTCTCTTTTGGGTGGTATGTCACAATTTGAGAGGACTGATTCATTTCTGAGTTGAT
TTCCCAGTATGTGAAGTCTCTGATCTGTTTGTGGTATATACTGCAGAATTTAATTTTTATTTATTTATTTAT
TTATTTAAGATGGCGTCTCGCTCTGTCGCCCAGGCTGGAGTGCAGTGGCGCGATCTCGACTCACTGCAG
CCTCTGCCTTCTGGGTTCAAGCGATTCTCCTGCCTCAGCCTCCTGTGTAGCTGGGATTACAAGCATGCAC
CACCATACCCGGCCAATTTTTGTATTGTTAGTAGAGACGGGCATGTTTGTGCATGTTGACCAGGCTGGTC
TTGAGCGCCTGATCTCAAGTAATCTTCCTGCCTCAGCCTCCCTTTTAAAATGCTGGGATTACAGGTGTGA
GCCACCGCACCCAGCCTTAGTTCTTTATGCATATACATACCAAGCTTTGAGGACTCTCACATGTTTCTATA
TATGTTCTGGATGTACCTAATATAATCTGTTTAGTTGTGAATGTCCAGCGTGTGGGGTTTCAAAGAGCCC
GTAGTGAATGTATATCTTTTGGTCTGAATCCTCTGATCTCTTTGTGGGTACATGTGGGTTCCTGTTCCTGG
ATATGAGCTCTATGATCTCTTTGTGGGTGTATGTCACAGGTTATGAGATCTATGATCTATTTCTGACTTTT
TTTCATAGCATGTCTGGTTTCTGATGTGCTTGTAAGTGTATGTTGCAGAATTTAGGATCCCTGGTCATATA
TAGTCTCTGATGTGTTTTGGAGTATATCCCATAATATGAGCTCCCTTATCTGTTTGTTGGTGTACATCTCA
AGAGTGTGAGGTCTCTGATATCTTTTTTTTTTTTTTTGAGACACAGTCTTGATCTGTCACCCAGGCTGGAG
TGCAGTGGCACAATCTCAGCTCACTGCAACCGCCACCTCCCGGATTCTAGTGATTCTCCTGCCTCAGCCTC
CCAAGTAGCTGGGACTACAGGTGCCCGCCACCATGCCTGGCTAATTTTTGCATTTTTAGTAGAGGTGGG
GTTTCACCATGTTGGCCAGGCTGATCTTCAACTCCTGACCTCAAATGATCCACCCACCTCGGCCTCCCAAA
GTGCTGGGATTACAGGCGTGAGCCACCACGCCCGGCCGTCTCTGGTATCTTTATGGGTATATGTTGCCG
GGTTTGAGGCCTGTGGTCTCTGTGTGGTTACGTGTCTAAGTTATGAGGTCTCTAATCTGTTTGCTGATGT
ATATCCCTGATCTTTTTTTTTTGTAGGTGTATGCCTAATGGTGTGAAGATTCTGATCTGTTTTCTGGTAAAT
GAACCGGGTTGAGATAAGTCAGACATCCTTGAAGGAGTTTTTTCAGAATGCGAGTTCCCTAATGTTTCCG
AATACATATCCTAGGATGTGATGTCCCAGGGCTATTTGTGGATATATATCCCAGGTGTGAGTTTTCTCAG
CCATTTATGGATATACATCCCAGGTTGGTAGATATCTTAGGCTTTTGGCATTATTTACAAGCATATGAGAT
TTCTGATTTGTATGCTCATATACATAACAGGTTGCAAGGCGTATTATTTGTTACAGTGTATACATCCAGGG
TATGAGGTTTCTGAGCTCTTTGTTGGTGTATGTCTCAGGGTCGAGGCTTTTGATCTGTTTGCAGGTATAT
GTCCCAGGATCGTAGCTCTCTCATCTCTTTGTGGGTGTGTACCCCAAGATATCAAGTATTTTTAATTATTC
TGAGTGTGTATTTGCCAGGATGTGAGATCGCTAACATGTTGTGGGTTCTTGTTGCAGGATTTTAGACCTG
ATCCATTTTGGTTATATTTCTCAGATGAAGTCTCTGATCTATTTGTGTGTGTATGTTACAGTATGTGAGTTT
AAAAAAAAAAACCTCGGCTGGGCATGGCGGCTCACACCTATAATCCGAGCACTTTGGGAGGCTGAGGT
AGAAGGATCACTTGAGCCCAGGAGTTTGAGACCAGCCTGGGCAACATAGTGAGACCCCCGTCTGTACA
AAAAATAAAAAATTAGCCAGGCTTGGTGGCGCATCTGTAGTCCCAGCTATTGGGGAGGCTAAGGTGAG
AGGATCTGCCTGAGCCTGGGAGTTCGAGGCTGCAGTGAGTCTTTATCATGCCACTGCACTCCAGCCTGG
GTAACAGAGTGAGACCCTGTCTGAAAAAAAAAAAAAAGTCTCTTTTGCTTGCTTATCAAGGGTATGAGG
TCTGTTCTGTTTGTGGGTGTATGTCCCAATTTGTGAGATCCCAGAACTGTTTGTGGGTGTATGTCCCAGA
ACACGTGTGTGTCTGCTCGCTCTTTAGATGTATGTCTCAAGGTAGGAGGTGTGTGATCACTTTCTAGATG
TATAACTCAGGATGAGGTAAGTGAGCTGTTTGTGGTGTGTGTCCCAGCATTTCAGGTCTCTGAGCTTTCT
GGAAGTGTGTGAAGTCTCCCATTGTTTGTGCATGTATGTTTTTGTTGTGTTGACTTAAGGCCCCCGAAGA
TGCCCATGTCCTAATCTTTGGAAACTGTGATGTGACTTTACATGCTAAAAGGTACCTTGCAAACAGCATG
GATGGAGCTGGAGGCCATTATCCTAAGTAAAATGACTGAAACAGAAAGTCAAAAACTGCATGTTCTCAC
TTATAAGTGGGAACTAAACAGTGGGTACACATGGATATACAGAGTAGAATAATAGACACCGGAGACTC
CAAAAGATGTGAGGGTGGGAAAGGGGTGAGGGATGAAATACTGCCTATTGTATACAGAGTACATTATT
CCGGTGGTAGGTACACTAAAAGCCCAGACTTCACCACTACACAAGATAGCCATGTAACATAACTGTACTT
GTACACCTAAATCTATAAAAATTTATATCTAAATATATAAAAATTTAAGTGTTTGCTTCAGCAGCACATAT
ACTAAAATTAGAATGATACACAGAAGATTAGTATGGCGCCTGTGCAAGGATGACATGCAAATTCGTGAA
GTGTTCCAGAAAATTTTTTTAAGATTAAATTATTTTAAAAATGTTTATTAAAAACATTTTTGAAAGGCCAG
GCGCAGTGGCTCATGCCTGTATTCCCAGCACTTAGGAAGGCTGAGGTGGGCGGATCACTTGAGGTCAG
GAGTTCGAGACCAGCCTGGCCAACATGGTGAAACCCTGTCTCTACAAAAAATACAAAAATTAGATGGGC
ATGGTGGTGCACGCCTATAATCCCAGCCACTCGGGAGGCTGAAGCAGGAGAGAGAATCGCTTGAACCC
GGGAAGTGGAGGTTGCAGTGAGCAGAGATGGTGCCACTGCACTCCAACCTGGGTGACAGAGTGAGAC
TCCATCTCAAAAAAAAAAATTTTTTTTAAAAGGTACCTTGCAGATGTGATTAAATTAAGCATCTTGAGATC
GGGAGATTTTCTTGGATTTTCACTGAATATAATGACACAGACTGAGTCAGAGACAGCGATATGATGACA
CAAGTGTTGGGGGTGTGGGTCCCAGCTCATGAAGTCCTTGCGCTGTTCATGGGAGTATATCCCAGGAGG
TGAAATCTCTAATTGTTTGTGCACACCAGTCTGTGGTTGGCTGAATAAAGCATCCCTCAAAGATGTCCAG
TGAAAGAGATATGACCAGGGAGCCCTAAATGATGGAAGGGAATGTGATGGTGGAAGCGGAGTCAAAA
AGGAGATGCCACAACAGAGGTAGACATCAGAGAGCATTTTGAAGATGAACTGCTGTCCGCTGTGAAGA
TGGAGAAAGGGGGAAGGGATGAAGGACTGCAGGCAGCTCTAGAACCCAGAAAAGGCAAAGAAACGT
GCTCTTCCTTAGAGTCTCCAGAAAGAAGACAGCTCTGTTGACACCTGACTTTAGTACCCTAAGGCCCATT
CTTGGGTTTCTGCCCTACACAGATGTAATATAATAAATAAATTTGTGTTTTTCACAGCACTAAGATTGTGG
TGATTTGTTACAACAATAAGAAATGAATACTGTGTCCCAGGTGCATGATGTCTCTGTCTGGTTGTTAGTG
CAAGATGTGAATCCCCAATCTGTTTGTGAGTGCATGTCTTTTATTTATTTACTTATTTATTTATTTTTGAGA
TGGAGTCTTGCTCTGTCGCCCAGGTTGGAGTGCAGTGGCACGATATCGGCTCACTGCAACCTCCGCCTCC
TGGGTTCAAGTGATTCTTCTGCCTCAGCTTCCCCAGTAGCTGGGATTACAGGCATGTGCCACCACGCCTG
GCTAATTTTTGTATTTTTAGTAGAGGACAGGGTTTCACCATGTTGATCAGGCTGGTCTCGAACTCCTGAC
CTCAGGTGATCCACCTGCCTCGGCCTCCCAAAGTGCTGAGATTACAGGTGTGAGGCACCACGCCCCGCC
GAGTGCGTGTCTTATGTGGCAGATCTTTCATCTGTGTGTGGGTGTATGAACCAGGGAGGAGGTCTCTGA
CCTGTCTGAGGGTGTCTATCCCTCAGTTTGTGTTCTCTGAGGTTTCTGCAGGTAGTCTCAGGATATGAGG
TCTCTGACCTGTTTGTGGGTGAATGTCCTAGAATGTGAGGTCTCTTGGCTGATTTGGAGTGTATGTTCCA
GTAAGTGAGATCTCTGAGGTAATAATAATATATATAATAACCTATATAATATATAATGTATATGTTCACAA
TATTTATAATTGTGCATTATAATTATTATATGATATTATATCATATCAATATATTAACATATAAGCATATGT
TAAGTATATATTATATATACATAATAGTATATATAATATGTTAACATATTAACATGAACAGATAACTATAT
AGTATATATTATATTTATATATAATATACCATAATATATTATATATTGTATTATATTGTGTAGTAACAATAT
ATATTATATTATGGTATATTTCACAATATACCATTCACAATAAATATTGTGACTACATTATGCATGGCTTG
AGGTCTCTGATATTTTTGTGGGTTTATATCCCAAGGATATGAACTCTGAATTCTCTGTGGGTGTATTCTGA
AGATGTCAGGTGTATACTCAGTCTCTGGCTGTTTGTTCTAGAATGTGAGATCTCTTCTTTGATTGTGGGT
GTATGTTACAGGTTGAGATCTCTGTTCTGTTTGTGGATGTCTTGGAGTGTGAGGTCTCTGATCTGTTTGT
GAGTAATGTATTGCAGGAATTGTGGTCTCTCATCTCCTTGTGGGTGTATTTCTCAGATGAGGCCTCCATT
CTGCTTTTGCCTTTATATTGCAGTATTTAAGTTTCTAATCTCCTCATGTGTGTTTGTCAAGGGTGTGAAGT
ATCTTCTGTTTGTGATTATATGTCCCAGTGTGTGAAGTCCCCACCCTCTTTGTGGGTATATGACACAGAAC
ATGTGCTTCACTTCTTTCTTGGATGCATGTCCAAAGGTGGGAGGAGGTCTCTGATCCCTTTGGAATTATA
TAACATAGGAGGTGGAGTCTTTGAGTTGCTTATGTGTGGATACCTCAGCAAATGATGTTTTTCATTGCTT
GTAGATGTATATCTTAGGTTGGTGGAGTGAAGGTTCCCATCCTTATATCCCTGGAATGTGTGACTGTGAC
CTTACATGTTAAAGGGTCCTTGCAAATGTGATTAAGTAATCATCTTGAGATGCGGGGACGGTTCTGGATT
TCCCAGGTGAGCCCAGTGTAATCACAAGGGTCCTTAGAGGAGAGAGGCTGGACAGTCCGAGTCAGAGA
AGCAGATGTGATGCCAGAAGCAGATATCAGAGAGATAGAGATTTGAAGATGAAATGCTCCTAGCTTTG
AAGGTGGAGGAAGGGGCCACAAGCCAAGGAACGCAAGCAGCTCTAGAAACTGGAAAAGGCAAGGGC
ACAGATTCTCCCCTAGAGCCTCCAGGAGTAATGCAGCTCTGCTGACACTGTGATTTTAGCCCTTTGAAAC
CCATTTTTTTCTTCTGACCTGCAGAAAAGTAAGATAATAAATGTGTGTTGTTTTATGACATTATGATTACG
ATTTGTTACACATTGATCTCTGTTTATTGGTACACATCCCAGGATGTGAACTCCATGATCTGTTTGTGAGT
GTATGTCTGATGGTGGAAGTCTCTGATCTGTGTGCAGTTGTATGAACCACATTGAGAGGTCTCTGAATGT
TGAGGGTGTGTGTCCCAGAATCCGTGGTCTCTGAGCTGTTTGTGTGTGCATGTACTAGGATGTGACATCT
TTGAGCTGATTTTTGGGAGTACATTCCAGTAGGTGAAGTCTTTGAGATATTTAAGGATTATGTTCCAGGT
TGTTACGTCTGTCATCTGTTTGGGGGGTATTTCCTGTGACATGAGGTTTCTGATTTGTTTGGTTACATATG
TACCAGGATGTTAGGTTGTTAGGTGTCTGGTCTGTTTAGGTTTATTTGTCTAGGGTGTGAGGTTTCTGAG
CTCTTTGTGGATGTATGTCTCAGGATTGAGGCCACTGATCTGCTTACTGGTAAAGAAATGTCCCAGTCCA
TGAGCTCTCTGATCTGCTCGTGGTTCTGTGTTATGAGATGTCGGGTCTATTTCTGGTGTTTTCCTAACCTG
TGAGGTCTCTGACTTCATTGGAGTTGTACATTGAAGGATTTGAGATCTCTGTTTGTGGGTGTGTGTCTCA
GGAGTGTATGGTCTGTGCATTGCTTGTGGAGTATATCCCAGGATGTGAACTTTCTGATCAGTTTGTTATA
TATGACTCAGGGCCTGAGGTTTCTGATCTGTTTGTGGATTGTGTTGCAGAGTTTGATGTCTGTGGTCTCT
TTGTGGGTTCATGTACAGGTTGTAAGGCCTCTACTCTGTTTGTGGGTGCATATCCCAGGATTTCAGCTCC
CTGACCTGTTTGATGCTGAATGGCACATAATCTGAGGGCTCTGCGCTATTTTTGGTATTTGGGGTCTCTG
ATCTGTTTGTGGTTACGTGTCCATGTTGTGAGGTCTGTTATCTATTTGTCAGTATATGTGCCAGGATGTGA
ACTCCCTGATATACACGTAGGGGTGTATCTGATGTGAATTCTGTGATCTGTTTGCAGGAGAATGAACCA
GGGTGGAAGGTCTTTAACCTCTTTGAGGGTATGTGTCCCAGAATATGAATTCTCTGGTCTATTTCTGGAT
TCATGTCCCAGGATGAGTGATCTCTGATCTAGTTGAGTATGTGTGTCCAGGGAAGAAAGTTTGTGAACC
CTTTGTGGTTTCAGGTCCCGTGGCCTGAAGCCTCTGATGTTCGTGGATACATGTCCAGGTTGTTATGTCT
CTGATCTGTTGGTATATGTCTCAGGATGTTAATTCCCTGATCTGTTTGTGGGTGGTGTCTGATGGTGTGA
AATGTCTGATGTGTTCACTGGTGAATGAACCACTGTGTGAAGTCTTTGACCTTTGTTGAGGGTTTTAACA
TAGGATCTGAATTTTCTGATCTATTTGTAAAAGCATGCCCTATGAGATGTTTGATCTTTGAGCTCTTTATG
GATGTACATCCCAGGATGTGAGTTCTCTGAGCTGTTGGGGCATACATGTTCTAATGTTCTAGGTTATCAG
ACCTCTGAAGTTTTTTTTTAGTTATATTTCCAAGCATATGTGATTTATATGCTGACATATATATTCATATAT
ATATGTAACAATATAGCTGTCCCTCAGTATCTGTGGGTTGGAGGAATTGGTTCCAGGACCCCTGTGGATA
CTAAAATCCGTGAACACTCAAGTCCCTTATATAAAATGGTATGGTATTTGCATGTAACCTATGTAAATTCT
TCTGTATACTTTAGATAATCTCAAGATTTCTTATAATACCTGATACAATGCTACGTAAGTAGTTGTTATACT
ACATTGTTTTTATTTGTATTATTTTTACTATGTTTTTAAATTATTTTTTTCCTCAGTGTTTTTGATCCAGACTT
GGTTGAATCCGAGGGCTGATAGTAGATATAACATATATATATATTGTGTGTGTGTGTGTGTGTATAATAT
ATATACCTGATTTGTTGAGGTGTGTATGTGTCCAAGCTGTGAAGTTATTGAGCTATTTGTGGGTTATTTCC
CTTGGTTTGAAAACCTCTGACCTATTTGGGAGTATGTCTCAGAATATAAGCTCTCTGATCTCTTTATGGGT
GTATGTTGCAAGATTTCAGGTCTGTGATCTATTTCTGGCTCTTTTCCCTAGCATGTGAGGTCTATGGCTTG
ATTATGGGTCTGTATTGCAGGATTGAGACCTCTGATTTGTTTATGGGTATCTAGCTCAGGTTGTGAGGTC
TCTGATCTGTTTGTGGCTTTATGTCTCAAGATGTCAGGTCTGTATACCTATTCTGCATATATATCCTGCAG
GTAAGGTCTCTAGGCTGTTTGTAGTTTCATGTTGCAGGATTCAAGGTCTCTGATCTCTTTTTGGTTGTGTT
TCTTACATGAGGTCTGTGATCTGTTTGCTAGTGAATGTCACAGTATGTGAGTGTCTCTTTTGTGTGTATTT
GTCAAGGATGTGAGGTCTAATTCAACATGTCCCGGATTGTAAGATACCTAACCTGGTAGTGGGTTTATGT
CTGAGAATGTTTCTTTCTGCAGTGTGTGTGTGTGTGTGTGTGTGTGTGTGTGTGTGTGTGTGTGTGTGTA
TGTCCCAGGTTTGGAGATTTGATCTGTTGGGGGTGCATTTTGCAGCATGTGGGGCCCCTGATATCTTCGT
GGGTATATATCCTATCATGTCAGGTCCATGTTTTCTTTGAGTGTGTATGTCCCAGATGGTGAGTTCTCTTA
TGTGTTTGTGAGTTTAATTCCAAGTGATTGAAGACTCTGAGTATTTTTTGGTATATGTCCCAGAATGTGTC
TCTGCTCTTTTTGGATGTCTGTTAGAGAGTGGAGGTGTCTTATCTATTTGGAAGTGTATGACTCAGGATG
TAAAGTCTGTGAATTGTTTGTGTGTGTATGTAGGTCTCTGGTAGCTAATGGTACCCCAAAGATGTGCACT
TCCTAATCCCTGAAACCTACAATTATGCTAATTTACATGGAAAGGGGGACTTTGCAGAGGTGATTGTGA
GGGACTTTGCAGAGGTGACTTTGTGGAAGTTAAGCATCCTGAGATGGGGATGCTTATCCTAGATTAACT
GGGCAGGCCCAATGTAATCACAGGTGTCCTTATATGAGGGAGGCGTGAAGGCAAGAGTCAGAGAGGG
CTATGTAGTGATGGAAGTACAGAAAAGATGATGGACAACATATACAGAGATCAAAGAGAGCTAGAGAT
AGATGGATACAAGGAGAGACAGATGGGGGAGGAGGAAGAAAGGGGGGTGGGGAGAGAGACAGAGA
GAGAGAGAAAGATTTGAAGGTGAAACAGACCTCTGGCTTTGAAGATGGAGGAAGAGGCCATGAGCTC
AGAAATGTAGGCAGCCTACAGAGACTGGATTCTTTGGCCAGGCGTGGTGGCCCACGCCTGTAATCCCAG
TACTTTAGGAGGCCAAGGTGGGTGGATCACGAGGTCAGGAGATCAAGACCATCCTGGCCGACATGGTG
AAAACCTGTCTCTACTAAAAATACAAAAATTAGCTGGGTGTGGTGGCGCGTGCCTGTAATCCCAGCTACT
CGGGAGGCTGAGGCAGTAGAATCGCTTGAATCAGGGAGTCGGAGGTTGCAGTGAGCCAAGATCGCGC
CACTGCACTCCAGCCTGGCGACAGAGCAAGACTCTGTCTCAAAAAAAAGAAAGAAAGAAGAAAAAGAA
AAAAGAAATTGGATTCTCCCCAAGGAACTCCAGAAAGAAGTCAGCTCTGCCAACACCTTGACTTTTACCC
TGTAAGACTCTTTGTCAGAAATGTAAGATCATAAGTTTTTTTTTATTTTTATTTTTTTGAGACAGAGTCTCA
CTCTGTCACCTAGGTTGGAGTGCAGTGGTGCCATCTTGGCTCACTGCAACCTCTGCCTCCCAGGCTCAAG
CGATTTTCGTACCTCAGCCTCCCGAGTAGCTGGGACTACAGGCGCCCGCCACCACTCCCGGCTTATTTTT
GTATTTTTAGTAGAGACGAGGTTTTGCCATGTTGGCCAGGCTGGTCTCAAACTCCTCGGCTCAAGGGATC
CACCTACCTCAGCCTCCCAAAAGGCTGGGATTACAGGTATGAGCCACCCCCAGCTGAGTGAGATAATAA
ATTTGCATTGTTTTATGACAAAAAGCTTTTAGTGATTTGTTTCAGCAGCAATAGAAAATCAATAGTGTGTC
AGGTTGTGCTGTCTCTATCTGGTTGTCAGGATGTGAACTTCATGATCTATTTGTGAGGGTGTGTGTCTGA
TAGTGGAGGTCACTGATTTCTGTGCAGGGTCTCTGATCTGATGACCTATTTGGTGGTGTGTGTCCCAGAA
TTTGTGCTCTCAGCTTTTGTGGAGGAGCGTATGTTCTAGGATGGGACATCTCCAATCTGATTTTGTGTAC
ATGTGCCAGTGTGTGAGTTCTCTGATCTGTTGAGGTGCGTATGTCTAGGGTGTAAGGATTCCAAGCTCT
GTGTGAGTATATTTCCTTTTGCTTGAAATTCTTGCCCTGTTTCTGGGTTTATGTCCCAAGATGTGAGACTC
CCTGATTTGTCTGTCTCTGTGTCATGGTGTGAAGCCTCTGGTATGTCTGCTGGTGAATGAAGCAGAGAGT
GAGGTCTCTCACCTCTTTTTTTTTGTTTGTTTTTGAGATGGAGTCTCACTCTGTTGCCCAGGCTGGAGTGC
AGTGGTGCGATCTTGGCTCACTGCAACTGCTGCCGCCCAGGTTCAAGCAATTCTCCTGCCTCAGCCTCCC
AAGTAGCTGGGACCACAGGCCCGTGCTACTATGCCTGGATAATTTTTGTATTTTTAGTAGAGATGAGGTT
TTGCCATGTTGACCAGGCTGGTCTCGAACTCCTGACCTCAGGTGATCCGCCCACCTTGGCCTCCCAAAGT
GCTGGGATTATAGGCGTGAGCTACCGCGCCCAGCCTCTCACCTCTTTGAGAGGGTTCTCTCAGGAAGTG
AGTTCTCTGATCTGATTCTGAATGCTTAGCCTAGGATATGAGGTCTTTGATCTGTTTGTGGGTATATATCC
CTGGCATGAATTTTCTGAGCTGTTTTGCCAATATTTGTGGGTACTTATGATATGCATATAAAGTTTCTGGT
TTGTGTGCTGGTATTTATAGTAGAAGTCAGGTTTTGTTGTGGTACATGTATCCACAATGTGGTTTTGGAG
CACTCTGTGGGAGACCTGTGATCTCCTCTTGGGTCTATACCAAAGACAGCAGGTCTCTGTACTTCTTCAG
GAGTATTTTCTGCATATGAGGCCTGTGACCTGGTGTTTCATGTCGTGGTATTAGAGGTCTCTGGTATGGT
TTCTGGTTGTGTTTCTCAGATAGGTTCTCAGATAGGTCTCTGATCCATTTGTTCGTGTGTGTCATCGTATG
TGAGTGTGTCATCTCCTCAGGGTGTTTATAGAGGGTGTGAGTCCCATTCTTTTTGTGGTTTTATGTCCAGT
TTGTGAGGTTCCTGACCTGTCTGTGGGTGTCTGTCAATGAATGTGTTTTTTTGTGTTCTTTGTGTCTGTGT
CTCAGAGTCAGAGATCTGGTATGTTCGAGGGTGTTTGTCCCAGAATGTGGGGTCCCTGATCTCTTCGCTA
GTGTATATCCCAGCGTGTCAGGTTCCGGTTCTCTTTGAGAATGTACTTGGCAGAGTGTGGTTCTCTTATG
TCTTTGTGGGTTTAATCCCCAGGGTGTGAGGACTATGAGCATCATTTGGCGTATGCCCCAGAGTGTGTG
GCTCTCTGATCTTTTTGGATTGATGTCCAGGAGTGGGAGTTCTCCGATCCCTTTGGAGGTCTATGACTCC
AGCTGTGAGATCTGTGAGCAGTTCATGGGTGAAAGTCCCGGGATGTGAGGGTTCTCTTTGTGCATGTAT
GTTCTGTTTCGTCAGATAATAGATCCCCAAATATGTCCTCACCTCATTCCTGGAGCTTGTTAATATGTTGT
CTTATCTGGAAAGGGGGCTTTGCAAATACATATTAGGTAAGCATCCTGAGATGGGGGCAGTTATCCTGG
ACAATCCAGGTGGGCCCACTGTCATCACAAGAGTCTTTATATGAGGAAGGTAGGACCTTCAGGGTCTGA
GAAGACTGTGTGTGTCACATCAGAGAGGACCATGTGACAACAGAAGTAGAGTCACAGAAGGTGATGCG
ATGACAGAAGCAGAGGTCAGAGAGAAATTTGAAGCCAAAACTGTTGAATCATCTGATCTCTTGGGAGTT
ATGCTGTAAGATGTCATGTCTACCATCTGTTTCTGGGTGTGTTTTTCAGCACATGAAGTGTGACTGGATT
ATGGTTGTATACTGCAGGATTTGAAGTCTCTGATTGTTTTTGTATGCTCCAGGATGTGAGAGCTCTGAGC
CATTTGTCCATGTACGTTGCTGGAGTGGAGGTCTCTGATCTGTGTGGGTGCATGTCCACGTTGTAAGGTC
TCTAATCCTTTTGTCAGTGTATATCCCATGATGTGAGCCCTCTGATCTGTTTGCAGCTATATTCGTAAGAT
AGTTATCTGTATGTTTCTGGGTATGCGTGTGTGTCGGGGTCTGTTATCTGTATTCTCTTTATGGGTGTGAC
CTCCCCTAGATGTGAGGTCTCTGATTTGTTTGTCATTGTATGTTCCAGGATGCAAGATTTGTGAATGGTTT
GTGGGTGTGTTTTGCAGGATTTTATGTCTGTGATCTATTTGAGGTTGTGTGTCATAGGATGTGAGGTTTC
TGATCTTTTTCTGGGCAAATTTCCTAGCATGCGAGGTCTGTGAACATTTTGTCAGTGTCCCAGGCTATGTT
ATCTCTGATCTCTTCACATGTATTGCCAAGTGTTTAATGTCTCTGATTTGTTTCTTGCTGTTTGTTCCAGGA
CGTGAAATCTCATACTGTCTGTGGATATGTGTCCCAGGGTGTGTGGTCTCTGATCTGTTTGTGGGTTTGT
ATATCCCAGTGTTTCATTACTGTCTGTGGATTTATTTCCCAGGATTTTTTAGTGTATTTCCTAGGATGTGCA
TTTCTCTGGTCTTTCTCAATGTATATCCCACAGTGGACAGTATCTGTTCCCTTTGTGGGTGTATATCCCTTG
GTTTATGATCTCTGATCCTTTGTGGGTTTATATTTTAGGATGTGAAGTCTCTATTGTGACTGTGGGTGTGT
GTCCCAAGACGTAAATTCTCTGGCCTCTTTTTGGGTTTATGTTCCAGGATGACAGGCTCTATGTGTATTTC
TAGCCTGCAAGGTCTATTACTTATTTGTGGGTGTAAGTCCAAGGGTATGAATTCTCTGATCTCTTTGAGG
GTCAGCGTACCAGAATGTCAAGTCTAGTTTGGTTATGGGTGTATGTCAAGATGTGAGCTCTCTGATCTCC
TTGTGGTTGTATGTCTCAGGATGTGAGGGTTCTGATCTGTTCCTGTATGTTTGTCAACAGTGTGTAATCTC
TGATCTGTTTGTGGCTCTGTGTCCCAGGTTGTGAGGTTCTGACCTGTCTTTCTTTCTTTGTCCCAGCATGC
GAGATCTCTGAGGTGTTTGGGTTTATGTCCCAGGAATTGAGTCTCTGGTCTCTTTGTGAGTGTGTGCCTC
AAGTAATATGTGAGGTCCCTGATGTAGTCTTTGGTTTATTTCCCAAGGTGTGAGTGCTCTGATCTATTCAT
GGGTATATGTCCTGGCATGTGAAGTGTCTGGTATTTTTATGTGTATATGTCTCAGGGACTGAGTTCTGTG
ATCTGTTTGTGGGTATAAGGCCCCAGATGTTAGTTCTCTGAAATATTTGTGGGTGTATATCTCAGCTTGT
GTGGTTTTTGATCTTTCTGTGAGTATGTCACAGGATGTGAGGTCCCCGTTCTTTTTGGGTGTGTGTTTCCT
AGAATTTGGATTTTCTGAGCTGTGTGTGTGTGTGTGTGTGTGTGTGCGCGCGCGCGCGCACATGTCACA
GGGTTGAGGTCCCTGGTCTGTTTGTTGGTGTATGTCCAGGATGTGAGTTCTCTGATCTCTTTGGGTGTGT
ACGTCCCACAATTTGAGGCCTCTGAATTAGTTGATACTCTGCATCCAGCATATTAATTCTCTGAAATATAT
TTTGGGGTCTATACCCTAAGTCTCAGAAGGATTCTGAATCCTGAATGCGAGGGTTCTGCGCTTTTTCTGG
TCTACATTCCAGGGGGGTGAGTTCACTGGGCTGTTTTGCGGACTGTGTTTACCTTGTGAGGTCTCTGAGC
TGTTTGTCCACTGGTGTCCTAGGATACGTGGTCCCTGAGCTCTTTTGGTTTCAAAGTCCCTTGACCTGTTT
GGGATATATTTTCAGGCGTGAAGTCTCTGTTCCTTTTCAGAGGGCTCAGTCCCGGGGTTTGATATATCTG
ACTCATTAGGGGTTTGTTCCAGGGTTAAGGTCTCTGAGCTTGTAATGGGTGTGTGTGCGCCAGTGAGTG
AGTTTTCTGAGTTGTTTGAAGGTCTATAGGTGTGAAATTTCTTAACTCTCTGGTGGTTCCTGTGCAGAGT
ATGATGTCTCTGAACTATTTAGCAGTCTCTCTTTTCAGGTGTGAGGTGTTTGTACCCTTGTATGAGAACTC
TGATGTATTTGAGTATCACCGCCAAACTGTTTTGAGCCAATGTCCCAACAAATGAGTTATCTGTGATGCC
GTGGGTTTTGTCCTAGGGTTGATTTTCCTGAGCAGTTTCAGGATCTGTTTCCAGAGTCTGATGTCTTTGTA
CTAATAATGTCTTTTTACTGTTTCAAGCCTCTGGAGTGCTTCACCCTCTGTGTCCCAAAGTGTCAGATCTCT
GATCTCTGTATTCCTTGGTGTTGAGGTCCCTCAACTGTTATGTTTCCAAGAGTGAGACAGTTCCTGTTGA
GGGAGTCTCAGTCCCAGGGTATGAGGTCTGTGACTTATTAGGGGTCTGGTTCTAGGGTTTGGGGTCTCT
GAGCAGTTTGGGGTTCTGTTATCAGTTTCTGACGTCTCTCTAGTATTTGGGGGTCTATGCCCGGGTTGTA
TGTCACTGGGTCACTCGGCTGTTAAGGGGTCTGTATCCTAGGGTGTAAAGTTTGTCAACTCTGAGCATTT
TTGCATTGTCTGTCTGCTGTGTGAGGTCTTTGGCATGTTTGTGGGTTTACTTTTCAGGTTGTGAGGTTAAC
TCTTTGAGGGTTTGTGTCCCATAATATGAGGTGCTTGAGCTGTGCGGGCATACTTAGCCAGGATGTGAT
ATCTCTGAACTGTTTTCATGTCTGTCTCCCAGGTTAGAAGTTCTCTGAGTCATTTGGGAGCCTGTGTCCAC
AATGTGAGGTCCTAAGCCGTTTTGGGTCTGTGTTCCAGTTATGAAGTGTCTCTACTGTTTGTGGCCAACT
TTGCACAAAGGTTTCAAAGCTGTTTGGTGATCTGTGTCCATGACTATCAGCCATCTGAAATCTTTCCGGAT
AAGGACAAAGGGCAAGTTCCATGAATGGTTTTGAGGACTGAGTCCAGGATGTGATATTCCTTAAGCATT
TAGAGGTCTGCGTTTAGAGTGGGAAGACTCCGAACTGTTTGGGGGTCTGAGCCTAGGATATGAGGTTTC
TAAGCTGTTTGGGGTCCGTGTCCAGGGGGTGTGATCTCAGTTCCTCAGGGTTTTTGTCATAGGGTGTGA
AGTCTTTAAGCTCTATAGAGGGTTTATGTCCACACTTTCAGGTCTGTGGGGCCTCTGAGCTGTTTGGGGA
TCTGTCTCGGTGTATAAAGGCTGTGAGCTATTTTGGGGAACTCTATGTCCAGTATATGACATCTCTGAAC
AGGAATCTGTGTCCAGGGTGTGAGCCTCTGAGTAGTTTTCGGGCCTGTGTTCCAGCATAAGTGATTTTA
GGGCTCTCTAGCATTGTGTCCGTGGTGCGAGGTCTCTGGGCTGTCTGGTGGTCTGAGAAGAGGTTGTGA
GGTCTCTGAACTGTTGGTCCATTTTTGTCCCAGTTTGTGAGTTCTCTGAGAAATTGGTGGATCTTTATCCC
TGATTGTGAAGTCTCTGAGTTTCCCGGAGGTCTGTAATCAGATTTCCACGTCTCTGAGCTCTTTGGGTGT
GGGGTTACAAAGTGTGAGGTCTCTGAAGTGTTTGGTATTGTCTGTTATCAATAGAATAGAAATATATTGA
TTACTAGGCTGCCTTGGCTTTGAACTGTTGAGGGGTCTGTGTCAGAAGGCGTGAGGTCATGTTTGAGCT
GTAAACAGATTTGTTTCCCAGGTATGAGTCCTGTGAGCAGGGTCTGTTTCCAGGCTGTGAGATCTCTAAG
CTCTTGGGGTCTGCTTCAAAGACTTGAGTCTAAGTTGTTTGTGGCACTATATTCACGTGTAAGGTCTCTG
GGATGTTTACTTGTCTGTTTTCTAGGGTGTCTCTGAGCAATCTGTGGGTCCCTATACAAGGTATGAGTTTT
CAGAACCATTTAAGGATCTGTTTTCAAAGGGCGAGGTATCTCGGGTGTTTGGGCAGTCTGTGTCCAGGG
TGTGCCAGCTCAGTTGTTTGGGGGTCTGTCCCAGGATACTAAGTCTCTGAGCGGTTTGGAAGTCAATGA
TCAGGGTGTGGAGCCTCCAAATAGTTTGGGTAGTGACATCTCAAGTTGTAAGGTCTTTGAGATGGTTGT
GCATCTCTGTCATCAAAGGAAAGGTCTCTGATGTGTTTAAGGTCTGAATCCCAGTATATGAGGCAGTATG
TGAGGTCTAAGAGCTGTTTAAGGGTCTGTAGCCCATAGTTTTCAGTGAAGCCTGTGTTACTTGCGGTGTG
TCCAATTATGAGAGCTCTGAGATATATGTAGGTGTGTATCCCAGGGTATGAGGTTTCTGAGAAGTTTGG
GGTCAATATTCAGGGTATATGGTCTATAAGGCCTTATACCCTAGATGCCCTGTGTATGTCCTGGTTACCCT
GTGTATGAGGTCTCTGGGCTTTTTGGGATCCTGCCACCTCTGTATGAGGTCTTTGAAATATTTGGGTATC
TGTCACGGCACGTGAGGTCCCTGAGCAAAAACAAAGAGCACTGGCTCCCTTTCAGATTGAGAGATGATG
AAAATGAGTAAGTATATCTGAAATGTGGTCTTCATTCTCCACTTTATACCCTGGCACCATATAGGTGCTCA
ATAAATATTTGCAGAAAAAGTTTCCCCAGCACACACAAACTTTTCATTTTGAATGCTGGAGGCAGATTTT
GGGTTCAAAGCCTTTAAGAGCAGGGAAGGTCCAGCTAGAATATTATCACATTTTAACCTTTCCATTGATG
TATTCCTCTGGTTATTTTTGTTTGCTTATGGCAAGTGATTATGGTTTTTCCATTTAGACAGCAGTGGATTTG
GTTTTCTCCCTCCCCACTTTGCTCCACTCCTTCTACCCTCCCCCCCACCCCCCCAGTGCTGTATAGTCCCTG
AGGCCTGGGCACTTGGTCAGGCAGGAGGTGGCAGGGTCTCAGCCAGACATTACCAGTCATGGCTGGTC
ACCACCAATCATGGCCAGTCAAGACTGGTCAAAGCTGGTTATGGCTAGTCTAAGTTGGTCAAAGCTGAT
CACGGCCAGTCAAGGCCAGTTACCAGAGATCATGGCTGGTCAAGGCCAGAAACCACTGGTCATGGCTG
GTCAGGATGGGTCAAAGCCAGTCATGGCCAGTCCAAACTAATCAATTATGGTCACAGCCAGTCACAGCT
GGTCCAGGTTAGTCACAGCAGGCCAAGACCAGTCAAGGCTGGTCAAAGCTGGTCAAAACCAATCCATTT
CAGTCCCTGCTAGTCCAGGCCAATCCAAGCCAGTCCAGGCTGATCACAGCCAGTTTAAGCAGTCAAAAC
TGGTCAATCCCAGTCCAAGCCAGTCCTGATTGGTCAAAACGAGTCGAGGCCAGTCACGGCTAGCTACCA
CCAGTCACAGCCAGTCATGACAAGTCAAAGTCGGCCAACACAGGTCAAGATCGGTCAAGTCCAGTCAAA
ACCAGTTACGGCCAGTCTAAACCAGTCAATTTAATTGCATTGTTTTGAGACAGGGTTTGGCTCTTTTCTGG
AGACAGACAGGCTGGAGTGCAGTGGTGAGATCTCAGCTCACTGCAACCTCCACCTCCCAGGCTCAAGCA
ATCTTCCCACCTCAGGCTCCTGAGTAGCTGGGACTACAGGTACAGGCACATGCCCCCACACCTGGCCAAT
TTTTGTATTTTTAGCAGAGACGTGGTTTCACCATGTTGCCCATGCTGGTCTCGAATTCCTGAGCTCAAGCG
ATCTGCCTGCCTCGGCCTCCCAAAGTGCTGGGATTAAAGATGTGAGCCACCACACCCAGCCTTAAGTGC
ATTTTAAAGTTTAAGTATAAAACAAACTTATTTTAGGTCAAGATGTTATATAGATGATGGTGTAAGTGAT
ATGCTGTGGGGGCTGAAATTGTGAGGATTAGAGTCAAATGGCTGAGGTTTGAGGAACATGTTCCTGGG
CAAACCCAGCTCCCAGCCTCTATCCCCTCAACCCCCACCCACACAAGCACCTCACATCTTGGTTTGTTCAG
TCGACAGTTCATTTTATTGATGCCCACTCCAGGCTGCATAGGGTTGAGGGGTGTACAAGAGGAGAACCA
GATTCAGTCCATGCCTGGAGGTTAGTCTGGGGGGCCGGCGGGATGGACACACAGACAGACACATAGAT
CTGGCATCTGATAGCAGGGCATACAGTCTCATGTAGGGGCACTCTGGGCACAGGTAGTCAGGAAGGAC
TTCCTAGAGAAGTCCTCACAGGGGGTGGGGGAGCAAGGAGCAAGGCAGCCATCAGAACCTTCGCCTTT
TGTACATTTGTAGGCAGGTGACTTTTGAGACCAGTGGAACCAGGGTGAGCACAAAGGGCTGGGGTCTG
GGAATGGCAGGCAGCAAGGGCTTCCCTCTCGCCTCGTCATCCCATAGTGGGGAAGCCATCTCCCCCACC
CCCAATTCTTGGCCTGTACTTTGCCTTCTGCCACATGGGCCCTTTCCTGGCATCCACCTCCGTGGTTTTCAA
GGCTTCCCTGAGACAATCCACGTGGACCAGGTGCTCTACCCAGTGCCTGGCACCTCATCACCACCCCATC
AATGCACATTCTTGTCCTAAGTGCTATCTACGGACTATCTAGATCTTGGTTCCAGCTCTGAGTGGTGTCAG
CGGTCATGACACCCCAGGTCAGGGCCCCTAGCAACCGCAGAGCTCCCAAGAGCCCTAGGTACCTTCACT
GCACTGTGAAGGCAGAGCTTGGTCCCAGCCTACCTCCAGGGCTGGCCAGCTCTGCACTGCCTTCCTCTCA
CTGCTCAGTGGCCCATGGACACCCACTTCCCTTCCAGTGGCATGTGGGCATCCACTTCTCAGATATGCAG
CCCTGAAGAAGAGACCCTACAGCTCTTAGGGCCAACACTACCCAGCACTGTAAGCACGAGCAGAAACCA
GCCTCAGCCCGTAGCCCAGGGACCCCCTTTCTTTCACTAGTACAAAGTTTCTGTGGTTGGGGAACCAGCT
TCATGTCGGGCTAGGCACTCTGGCCTCTGATGTCTGCCTGCTCTGGGGTTGCTTCCCCATACCTTTGGGG
CAGCAGGGAGGAGGCCTGCAGCAGACCCCTCAATCTCCAGGCAGAAGCAGGTGCAAAAACAAATATAT
TCTGGGGCTGGGTTGGGACTCACCCCTTGGACCTCCTTAATAGGCCATGAATCCCAACGACAGAGAGTC
TTGATGTTGAAAAACTATTTCTGAAGGTGGGTGGGGGTTTGTAAGTGCAACCCACAGATGGGGCTGGG
ACAGGGAAAGGGGACTCCTGGAGTCGGTCCTCACCACGGCCACAGCCCTGAACGTAGAGCAGGTAGGA
GCAGGTGGGGGCAGGTGGAGGCAGGCGAGGCCCTGGCAGTCATTCTCGACAGGAGGGCAGCAGTGG
GGGAGGTGCATGTAGCTTCAGGCCCCCTCAGTCAGTCTGGCCTCAGACTTCTAAGGGCTAGAAGAGGTA
TTTGCGGCAGGACTCAGTCCCACACTTGCATTCAATACGGACCCGCTTCTTAGGGGAGCCAGGGAGCCC
AGCCAGGCCAAAGTTGGAGTCCATGCGGGTGCTCTCCATGTCCACGGGGTCCACTGTGGAGTCACAGCC
AGGGAGGAGAGGAGGGAAGGAGGCAGGGAGGGAGGGGGAGAAGGAGGGAGGGAGGAAGGTAGG
GAAGGAGGCAGGGGACTTGTTAGAAGGCCTGGATGAAGGGTCCCACTACCCTTTATACCATGAGACCA
AGGATCTTTGGGGCTGGGAGCAACTCTGGGATTTGAGTTGGGAGATCTTAGGGTTGGTGGTTTGGGGC
TAGGGCCAGGGCTCAGCACTGGAGGTTGTTCAGGCCAGGGGTTGAAGAATGCTGGGGGAGGCAGGGA
GCCGTCAAGGAACCAGGAGGGTCGTTTTAGGTGTGTGGGGGGGGGCAGGGGCAGGAATAGAGGGCC
ACCTGGGCATCCAGAGGTACATCTGAACTCTCCTGCCAAAGACCTGCCCTTGAGGGTCTCAGAGGTGGC
TAGCTTATCAAGGAGGGGACTGGCTGGAGGCCACAAGGGTGTCCAGAGGCTTGGGATGAAGAAGGCC
AAAGGGAGCTAGGAGTGGGGAGGGGCTGGGGGATGAGGGAAACTCAGTCAAGACTGGAGTCAGGAG
TTTCTTAACTCTCCAGGAATCTTGGGAGAAGTTGGTATGAATGCCTCAGGGTTAGGGTTCAGGGGCTGG
GAATGAGGGGACTCGAGGGGTAGGCAGGGGTCTAGTAGTCCGAATGGGGGACTGGGAGTCAGGAAG
AGCATCTTAAAGCTCCCGCTAAAGAATTTTCCTTGGTTCTCTGAGTGACTGGTGTGCCTTTGGGAGGGGT
CCTTGTGTCCCAAGTCACAGTGCGCCCCTGCCCCCAGTCCCTTGCCACCCCCACCTTGCATGTTGTAATCA
AAGGTGAGCTCCTCGCCTGCCCGGATGGTTCTTGTGGCAAAGAAAGCGATGCGGGGCAGCCGCTCGTC
AAGGTTGTCTATGAAGACGTTGTACACCTGCAGGTTGGGGTCACACTGGGCGAGAAAGAACAGTTGGG
AGAGTGAGGAGGGGCTCCAGCCTGCCCCTCTCGACCCCTTGCCCGCCTGCCGGGGGTACCCACACTGTG
GTTGACAAAGTGGGAGATGTTGCCATAGTAGGCGGCATCCACGGTGTACACGTCCTCCACGTAGTCCAG
GTCAAAGAGGTAGGTGGCGCCCTGACGGTCGTAGATCTGGCCCCGCCGCTCTGCCTCCTCTGAGGTAAT
GATCTAGGGAAAGGGCCATGGGTAAGGGGAGATTGGCCGCGAAGTCTCAGGAAAGCCGGCACTGCTC
CTGACTTCTGAAGGACAGGGAGCAGGAGGCACCCCTGCCCGTGGACACACATGGGCAGTGACGCAGA
GAGTCTGTCGGCCCTTCCTGAGGAGCTGGGCCTCAGCTGGCAACTGCCTGGCCCGCCTCAGGCCTCTGG
CTAGCAGGAGGAGTCCCTGGCGGTGAGTGCAGGCTGGCCTGGCTGCAGGGCCCAACTGGGGCCACGC
GAGCACCTCCTGCCGCCACCCAGCAAGAGCTGCCCAAGAACCCCTGGAGCCTCCAGTGCCTTGGGACGG
AGCAGGAAGGTGATCAGGAGAGACCAAAGCAAGGGCAAGAAAGAAAGTGACCCAGGGTGCCACAGG
GAGAGATACTTACTGATCCAGGCAAGGGAGCCTGAGCCCATGCAGGACCCAGCAGGGAGGGCGTGGA
GAGAGGGGAAGAAGAGACCCAGGCCATGGGGAAAGGGGGCAAGGGCCTCAAGACAAGAGAGGTGG
AGGCAGAGGCAGAGGGAGGCCCAGGTTCAGAGAGCCAGAAAGAGGCTGAGGCCACACACTTCAGAGA
CAGCAGCAGAGGAGGAGGAAAGATGAAGGGAGAGCACAGTGAGAGAAGCTCTCCCTCGGCCCAGGGC
TGCGCAGTGCCAACTGGTGCTTCAGACGGCAGTGGGTGCCTCCTCCCCAGCTGGTTCCCTACCTGTCCTA
GGAAATGGGAAGCTTTGCTGCCATCCTAGCCCCAGCCTGTCCTTGCCTCCCAATCCACGCCAACAAATCT
CCGGCCCCATGGCCCCTCTTCCCTGAGGCCAGCAACGGCAGCCTCCTGCCAATCCCCAAGCGTAGCTGAC
CAGGCCCTTCCCTGGGTAGGGGTCCCACTGTACCTCAGCCAAGAACAGTGCACAGCACACAGGAGACAC
ATGGGGACTGCTGTTTACTTTGATTCTGCTTCCTGGCTTAGCAGCCTGTCCTCTGTCCAAGTGGCGGCCCT
CCAGGTAGCTCCAGTACTCCCCCGCCACACTTCTTCCTTCCCCTCCCTTCTCCAGAGACCCCTCTCGGGCTT
CCTTCCTTGCTCACCCTCATGTGGGCCGGTGACCATGCCCTTCTGACTTGGGAATCCATCTCCTTAGCTAG
GATGGCCACACTGGTGTTCTCCCTGGCTGTCCCCTCAAGGCTGTGTTCCATGCCAAAGGCTGACTAACAA
TGCCTTGAGGAAACCAAGATTTACCCTGCCTCGGGCCACACTACCCACTGCCCCGTGCTCCAGTTCTGTT
CCTTGAGGAACCTGGCTCCCTTGCCAGGGTTTTTATTGAGGCCTTCCTTGTGCCCAGTCCCAGCATGCAG
GGCTCACGGTCTGATCAGGGAGACACAGGGACCAGGCCCGGTCCACTCTCAGGGGTCACAGGTGGGTA
GGGGATACACAGGAACCAGGATTGGTCCCTGCCCTCAAGGGGTCAAAGTCTGGTCACAGAGAGACAGA
TTCACACACCCACAGTGTAAGACTGTGATCAGGAAACAGTGCTTAGGGCTTGAGGGGGAATGGAGAAG
AGACGGATTCTCTAAAACGAGTTGCAGATCATAAAAGGGCTTTACCACCTGGTGGAAGAGTGGGGACT
CTCTCCTAAGGGCCACAACATGAGGGTGGAATATGTCAAGATCAGTCTCCCTTCACCTCCTGGAGAACA
GTCTTCCTTGTGGCATCAGCTCAAACAGATGGGACCTCTCTAGGCAGCCTTCCAAGCTTCCCGTTCCTCCC
CTACCTCAGAAGCTGGCCACTTGTCTGATCAAGTCCTGGGATCAAAGGGCAGCCTCTCCCAGGCTCACA
GGCCTAGTGGGTGGGGTATGAGCCATCTTTTTCACCGTAGGCCCTCCTTGAGCCAGGAGGAGTGCCCAC
ATCCTTCCTAGTCTCCATCTTCATGCCTATTCTGCCACTACCTGCTCTCCCAGTCTCACCAGGAGTGAGTCA
TACTGCAGGCCCTCCAGGGCACAAACACTTCTTGGTTCCTTCTCATCTTCTTTTCAGTCCCATTCTCTAATG
ATTGGTCAACCCTGAAAGCTTGGGAACAGATGGGCCTGTCTCTGCTCCCTAACAGCTCTCCAGAATGAAT
GGAAAAGACAGGCTGTAAGCTGGCAGTTCCGAGAGGGTGCTGACGTGAATGGGAACTAGAAGACTTG
GAGTCAGCTGCTGCTCTGCCCCCTCCTGGATATGGGATCCTGAATGTTCTCTCTGTGCCTGTTTCCTCAGC
TAAAAAAAAAAAAAAAAAGCTACAGCCTGGCAGGGTACCAGTTTTAGATATTATTTGGGCCTTTATTTTT
TTATCCATTGTACTACTTTTGTGATTTTAATCAAGTTATTCTACCTCTCTGGGCCTCAGTTTTCTCATCTGCA
GAATGGGAATGACAGTGACAAAATCCCAACCTGACAGGGGCTGTTGTAAAGATCCATACAAGATCATG
GACGTGAAAGGTGTTCCTGTTTCTGATAGGAGAGTGAGCAATGTTAGGAGTGGTTATGAGGACCAGTT
ATAAATGTAAGGCACACCCTTAATTTTATGTGCCCTTTACCAAGAAAGGAAAAACTGCTGTTAATTATAA
ATGATACACCATGATTTAATTAAGACATCTTCATTTCCGAGATGTTAAAATAGGAAGAAAATGCATGTTT
TAGAATGGATAAAATAGGCTGGCATTGTCTCTTGGGTGAGGATGGGAATGTTCTGCAGGAACGTGAAA
GTTCTGACCCAATGTGGGAGGCTGCAAAGGAGGGAAGGGCAGCTCCCGTGGAGGGAGCTCTTGGTAC
AGACTGTCAGTACATGTCCCAGCTGAAGAAGGACAGTAGATAGCATGGTGTGCAGATAGGCCCTTGATT
CCGTAACACCCTGGCAGCATCTGGGACGGCACTACAGAATCAAGTAAATTAGAACTGCTGCAACGAAAT
GTATGACTATTACCCCAAGTAGGATAAATAAAAACTACAAATAGGACCCTATCCGGTCAGGCTGGGTCC
TGTAGCCAGATGGGTTACAAAAAAACTTGGTCTTCCAGACATTTTGGATTTGCTGAAGTTTGAACTATGC
TGATAACATTTGTATCAAACTTACTGTGTACTGAACACTGTTCAAGTGCTTTTTCCAAGTGCTTGATACAT
AGTAACTCATTTAATTTTTGTAACAAGCCTGTGAGGTGAGTGCTATTATTGTTCCCATTTTACATAGTAAA
TGCAGGCACAGAGGTTAGTGATTGCTGATGAGTAGTGGAGCTGGGATTCAGCCTCGGGCAGTCAGGAT
CCAGAGTCCACCCTCCTAACCATTTGCTATACTGTCTCTTCAGAGGGGAATGCGGGAAGATGAGTTTGGC
TCTTTCCGGAGGCCTGTGGCACCATCAGGAGGACCCATGGGTCTGAGGGTGCTGGAGGGCGGTCAGGA
AATAGTGCAGCAAGAATGTGTGGAGAGCAGGAAGGGTGAGGACAGGGCTGAGAGGATCCCTGGGAG
GATTAAAGACACTGACACTAAGGAAGAGCAGCCAGAAAAGTCTCAGGCCCAGGAGGGTCTGAGGCTGC
CCTTGGGGTGTAGTCTTCCTCCTCGACTGTCAGGACCCTGTGTGGTAGACAGTGTTTCCTCCAGATGCCA
GTACAGTGAGGCTGAGATACTTCCTGCTCTGTGCAGCCCCTAGGACGGTGGACTGTCTGGGTCCTATGG
GGTCTGGACATCTCCTCCAGAGAGTTTGGGTTCCTTCAACCTCTGGACACTTCCTCCAGAGCGTCAGGAC
ATTGAGGGCATGGACACTTCCCCCGATACTGTCAGGGTGCCCTTGGGTGTAGACTAGATCTCCAGGCTG
TCCGGATCCCTCTGGGGTGAGGACACTTCTTTTTCCTGTCATGACAACATGGGTGTGAACACTTCCTAAG
TCAGACCACCAGGTCCTCTAGAGTGTGGAGAGCACAAGCTCTTCCTCCAGGTCACTGGGGTCACTGTGG
GACATGGACTTCCTGCAGATTATCAGGACTCCTTCCCCCGCCCACTGTCAGGAGCCCTCAGGGTGTGGAC
ACCTCCCCACCTCCTCCTTCCAGATGGCCAGGTCCTGTGGCATGTAGACATCTCCTCCAGCAGGCCAGCA
GGGTTATCTGGACAGGGTGTGGCTGTCTCCTTGCCCAGACTGTCAGGGTCCTCTGTGGTGTAGATTGTTC
TACCCAAGATTGTCTCTGTCCCTGTCAGATGTGACCCTCTGTACAGTTTCTCAGAGTAGGAGCCTCTCCTT
GTCATCAGTATCAGTGCTACAGTGTGGACACCCTCATGTGAGTGTTCCTCAGGCCAGTTTCTGTCACTTTC
CAACTATATCACACCCCTAAGGGACCTATGGCTATATATAGAGGTATCTGGGGTTGTCACTATGACAGG
GCACACTCATGGATTTAAATGGTCAAGGTCCAGGGATGCTAAATGTCCCCCAATGCATGAGACAGTTCT
ACATAACGAAAAATCATCTCATCCCAAATGCTAACTGCATCCCATCTTAGAATGCGACATTTTTCTGTTGT
AGATACTTGCTAGTTATCTAGTTGCTAGTTATGGCTGTGCTTTAGCCTTGTGCAGCTGGGCACATGGCTA
AAGCACATCCTGTTGCCTCTCTTGCATGGAGGTAAGGCCAGTGACTCAGTTCCAGCCAGTGGAGCATGG
GCAGAAGTGATGGGTGCTGCATCTAGGCCTGGCCCACTAGGATGGGCCCCTTCTATCCCCCACTTGGCC
CGCCTGCCAGGCAATGCTGAGGATCTGGTAGTGACTCCAAGGCTTCAAGGCACAGCAAAGGCACAGGA
GGGAAAGAGCCTAGGTGTCCCAGTTGCTGTGTGAAAGGATACCCACTCAAACCCCCACAATGGACTGTT
CCATGAGCAAGCAAACTTGTAATGTGCAAAGTCACTAAGATGAGGGGGTTGTTTTTACAATGGCTGGCT
TGCCTAATACACTCGGCCTTCCCCAGTGTCCTGAAAGTCTAAAGGAGTTAGCCAACCTTTGTCCAAGGCA
TGTCTCATCCTACTCCAGACTGCGTTCCTTCTGTGCAGACTGGTAAAACAATGCTCCTCAGGCAGATACT
GCTTGGTGATAAGATGGGCCCTTGTGAAATGAGAAGAGGAAGTGCCTTTCCAGCTGATGCAGCCCTATG
CCAGACCACATGGCCTGTTACAAGGAATGCTAAGGCTGCCTGGATCTCAGCTCCCACTTTTCTTCTTTGCT
AAGTACCCTGATAAAGTCAACCCAGTGCAGAAGTATTTTGGTGTTCCTGTAACCACCAGCATGGCCATGC
TGGTGTGTACCAGCTTGGAATACTAGCCCCTGCCCAATGTCCCTGTGACACTCAGATCAGTCTCCCAGGC
CTTTCCCATAGGGAAAGCAGGGAGTTTCAGACACACAGTGAGGAGAGCCGGGTGCACACTGTGAACAC
AGGGTGGGAAGAGCTGCACACACACACCGGGTCCCATCTCCCTACCTCTCCCACGTACTCCATGACGAA
GCTGTTCTTGCGAATCTTCTCCAGGGTGCGGACGCCCCAGCCACGCCCATCATCCGTGCGGAAGATGCA
GAGGTCATATCGGATACCCTTCTGTACCACACGATTTGGGCAGTCATAGCCGCAGCGGCAGCGGGAGTT
GCACTCGTAGATGGGCAGCCCGGCTCGAAGCCGCACCTGGCCCTGGTCATTGTAGGCAAACTTGTGCAG
TGACGCCCCCGGGCAGCAGCCTCCAGTGGGTGCCCACAGACAGTCCTGGCACTCGCAGCCCACAGCCAC
CTGGTTGAGGGTGATGCCCTCACCAACACGGTACTCATTGATGTACACGAAGGCCCGCGGAGGGCCGTC
CAGGTCCACCTCATTCTCTACAGTGATGCGTCCCAGATGGCTGCGCTTGGCATTGAGCTCCTGCTCCCAG
CGACGGAGCGCCCGCCTCTGCTTGGCCTTCTGCACCAGGTAGTTGGCCAAGCTTGGGTCCAGGTGCCGG
GGGGTCTTTGACCGGTGGTGCCGCCGGAGCAGCTCCCTTTCTAAGTCCTTGTGGAACTGCTTGAGGATA
CGCACACACTTGAGATTCTGCCGTGGCTCCCAGGTGCTCTCTGAGTCTGGATATCCACGCCATTTCACCA
GGTAATATTCCTGTTCCTGTTGGGGGTGGGTAGGGGGACGGGGGTACCGTAAGTAGTGAGATCTTTGG
GCCAGCCTTCATGGGGCTTCCATCCCAAAGCTGATGGATAACACCTACCCCACCCCACCCCTTTTCTGGA
ATGACTTCATGCGGTGATTAAAAAGCATGGGCTCTAGAGTGAGGATTACTAGGTTCAAATCCTCCTCTCC
CCCTTACTGTCTGTCCAAACTGTTCCAACTTTTAATTAATCTCCCAAACCCACTCCTCTCCTTGGTCTTCCCC
ATCATCAGCTCCCTCCCTTCTAGCTGTTCAGACCGGAACTCCTGGAGTCATCCTTGACTTCTCTTTTGTTCA
CACTTCCGACACCCACCCTCTGCACATCCCGTTGGCCCCACCTTCAAAATACGTCCAGTGACAAACCATCA
TCACTGCCACCACCCTGGCTCACCTGGACCCTGACAAGTCCTTGCCTGTTCTAGGCAGCTTACATGTATTA
TTAACTAACTTAATCCTCACAACCCCACATGAGGTAGATGCTCCTAGTTATCCCTCTTTTACGACAAGGAA
AATGAAGTACTGAAGGTTTGGGTAGGTAGCCTGGGGTAACACAACCAGGACTGGAACCCAAGCATGCT
GACTCCAGTTTGTTCTCCTAACCATCCTGCCACTCCCCCTTCTGGCAATAACTATCCCCATTTCAGTTGTTA
GGGGATTAAATGAGTTTCCGAGAGGAAACGCCTGGCACACTGAAAGTGCTCAATAGCCATTAGCTATTG
ACATTTCCACTCCAGGGTTCAGCATCTCAATCTCTGCAATTACTTTGTATTCCTAGGGAGATGGGAAGAG
ATTTATTTGGGAATTCATTCCGGCTCCTGCCCAGAAAGCTACTTAGTTTCCCTATCTGTAACATAGGGATA
AAAGGGTCTATTCATCCTGTGTTTTCAGGGTCAAAGGAGAAAATTCCCTTTGGAAACAGATGTGGGCAG
TTGGGGACAAGAGGGCAGGACACTAACTTCCTTGTGACCTGTCCCCTCCCAGAGCATGGTCACCCCAGA
CTCACGCGGATCTTCTTGTAATCGCACAGGTACTCGACTTCAAAGTCATAGAGGTTCCTCTTAGAGATAC
CGAGGGCAGGGCAGGAGAGCTTGGCCAGGCGGCACAGGTCCTGCAGCTGATTCCAAGAAGACTTGCA
ACA
SEQ ID NO: 6
lncRNA AF196970.3 cDNA sequence
CGAGGCAGGGCTTTGGCTACTGGAGATCGTAGGTTCGAATCCCGTCTGGGAAGTTCAACTTGTGCACCT
GTAAAAGAAGCTGGCATTATTGGCTTGTACTCAAGGGCTGGCACAGAGTGTGTCGGGGTGCGGACGCC
CCAGCCACGCCCATCATCCGTGCGGAAGATGCAGAGGTCATATCGGATACCCTTCTGTACCACACGATTT
GGGCAGTCATAGCCGCAGCGGCAGCGGGAGTTGCACTCGTAGATGGGCAGCCCGGCTCGAAGCCGCA
CCTGGCCCTGGTCATTGTAGGCAAACTTGTGCAGTGACGCCCCCGGGCAGCAGCCTCCAGTGGGTGCCC
ACAGACAGTCCTGGCACTCGCAGCCCACAGCCACCTGGTTGAGGGTGATGCCCTCACCAACACGGTACT
CATTGATGTACACGAAGGCCCGCGGAGGGCCGTCCAGGTCCACCTCATTCTCTACAGTGATGCGTCCCA
GATGGCTGCGCTTGGCATTGAGCTCCTGCTCCCAGCGACGGAGCGCCCGCCTCTGCTTGGCCTTCTGCAC
CAGGTAGTTGGCCAAGCTTGGGTCCAGGTGCCGGGGGGTCTTTGACCGGTGGTGCCGCCGGAGCAGCT
CCCTTTCTAAGTCCTTGTGGAACTGCTTGAGGATACGCACACACTTGAGATTCTGCCGTGGCTCCCAGGT
GCTCTCTGAGTCTGGATATCCACGCCATTTCACCAGGGTCAAAGGAGAAAATTCCCTTTGGAAACAGAT
GTGGGCAGTTGGGGACAAGAGGGCAGGACACTAACTTCCTTGTGACCTGTCCCCTCCCAGAGCATGGTC
ACCCCAGACTCACGCGGATCTTCTTGTAATCGCACAGGTACTCGACTTCAAAGTCATAGAGGTTCCTCTT
AGAGATACCGAGGGCAGGGCAGGAGAGC
SEQ ID NO: 7
lncRNA AF196970.3 exon 1 cDNA sequence
CGAGGCAGGGCTTTGGCTACTGGAGATCGTAGGTTCGAATCCCGTCTGGGAAGTTCAACTTGTGCACCT
GTAAAAGAAGCTGGCATTATTGGCTTGTACTCAAGGGCTGGCACAGAGTGTGTCGG
SEQ ID NO: 8
lncRNA AF196970.3 exon 2 cDNA sequence
GGTGCGGACGCCCCAGCCACGCCCATCATCCGTGCGGAAGATGCAGAGGTCATATCGGATACCCTTCTG
TACCACACGATTTGGGCAGTCATAGCCGCAGCGGCAGCGGGAGTTGCACTCGTAGATGGGCAGCCCGG
CTCGAAGCCGCACCTGGCCCTGGTCATTGTAGGCAAACTTGTGCAGTGACGCCCCCGGGCAGCAGCCTC
CAGTGGGTGCCCACAGACAGTCCTGGCACTCGCAGCCCACAGCCACCTGGTTGAGGGTGATGCCCTCAC
CAACACGGTACTCATTGATGTACACGAAGGCCCGCGGAGGGCCGTCCAGGTCCACCTCATTCTCTACAG
TGATGCGTCCCAGATGGCTGCGCTTGGCATTGAGCTCCTGCTCCCAGCGACGGAGCGCCCGCCTCTGCT
TGGCCTTCTGCACCAGGTAGTTGGCCAAGCTTGGGTCCAGGTGCCGGGGGGTCTTTGACCGGTGGTGCC
GCCGGAGCAGCTCCCTTTCTAAGTCCTTGTGGAACTGCTTGAGGATACGCACACACTTGAGATTCTGCCG
TGGCTCCCAGGTGCTCTCTGAGTCTGGATATCCACGCCATTTCACCAG
SEQ ID NO: 9
lncRNA AF196970.3 exon 3 cDNA sequence
GGTCAAAGGAGAAAATTCCCTTTGGAAACAGATGTGGGCAGTTGGGGACAAGAGGGCAGGACACTAA
CTTCCTTGTGACCTGTCCCCTCCCAGAGCATGGTCACCCCAGACTCACGCGGATCTTCTTGTAATCGCACA
GGTACTCGACTTCAAAGTCATAGAGGTTCCTCTTAGAGATACCGAGGGCAGGGCAGGAGAGC

| | Target | | | | | |
|---|---|---|---|---|---|---|
| shRNA | Sequence | SEQ ID | Loop | SEQ ID | guide | SEQ ID |
| shRNA #1 | GTTGCAAGTCT | SEQ ID | TTGATA | SEQ ID | CAGCTGA | SEQ ID |
| | TCTTGGAATCA | NO: 13 | TCCG | NO: 19 | TTCCAAG | NO: 20 |
| | GCTG | | | | AAGACTT | |
| | | | | | GCAAC | |
| shRNA #2 | AGTCGAGTAC | SEQ ID | CTCGAG | | TAATCGC | SEQ ID |
| | CTGTGCGATTA | NO: 14 | | | ACAGGTA | NO: 21 |
| | | | | | CTCGACT | |
| shRNA #3 | AGATCCGCGA | SEQ ID | CTCGAG | | AATATTC | SEQ ID |
| | ACAGGAATAT | NO: 15 | | | CTGTTCG | NO: 22 |
| | T | | | | CGGATCT | |
| shRNA #4 | GATCCGCGAA | SEQ ID | CTCGAG | | TAATATT | SEQ ID |
| | CAGGAATATT | NO: 16 | | | CCTGTTC | NO: 23 |
| | A | | | | GCGGATC | |
| shRNA #5 | GCAGTTCCAC | SEQ ID | TCAAGA | | TCTAAGT | SEQ ID |
| | AAGGACTTAG | NO: 17 | G | | CCTTGTG | NO: 24 |
| | A | | | | GAACTGC | |
| scramble | CCTAAGGTTAA | SEQ ID | CGAGCG | | AGGGCG | SEQ ID |
| | GTCGCCCTCG | NO: 18 | | | ACTTAAC | NO: 25 |
| | | | | | CTTAGG | |

SEQ ID NO: 26 shRNA #1:
GTTGCAAGTCTTCTTGGAATCAGCTG-TTGATATCCG-
CAGCTGATTCCAAGAAGACTTGCAAC
SEQ ID NO: 27 shRNA #2:
AGTCGAGTACCTGTGCGATTA-CTCGAG-
TAATCGCACAGGTACTCGACT
SEQ ID NO: 28 shRNA #3:
AGATCCGCGAACAGGAATATT-CTCGAG-
AATATTCCTGTTCGCGGATCT
SEQ ID NO: 29 shRNA #4:
GATCCGCGAACAGGAATATTA-CTCGAG-
TAATATTCCTGTTCGCGGATC
SEQ ID NO: 30 shRNA #5:
GCAGTTCCACAAGGACTTAGA-TCAAGAG-
TCTAAGTCCTTGTGGAACTGC
SEQ ID NO: 31 Scramble sequence:
CCTAAGGTTAAGTCGCCCTCG-CGAGCG-
AGGGCGACTTAACCTTAGG

1. A cell comprising a first nucleic acid that is:
(a) a heterologous nucleic acid that expresses an RNA comprising the nucleobase sequence of [SEQ ID NO: 1] (lncRNA AF196970.3) or a fragment thereof capable of inhibiting expression of SUV39H1 in the cell;
(b) a heterologous nucleic acid comprising the nucleotide sequence of [SEQ ID NO: 5] (genomic sequence encoding AF196970.3 (ENSG00000232828)) or a fragment thereof that expresses an RNA capable of inhibiting expression of SUV39H1 in the cell;
(c) a nucleic acid that expresses an RNA comprising the nucleobase sequence of [SEQ ID NO: 1] (lncRNA AF196970.3) or a fragment thereof capable of inhibiting expression of SUV39H1 in the cell, said nucleic acid operatively linked to a heterologous expression control sequence (e.g., promoter, enhancer, other regulatory sequence); or
(d) a nucleic acid operatively linked to a heterologous expression control sequence comprising the nucleotide sequence of [SEQ ID NO: 5] (genomic sequence encoding AF196970.3 (ENSG00000232828)) or a fragment thereof that expresses an RNA capable of inhibiting expression of SUV39H1 in the cell, said nucleic acid operatively linked to a heterologous expression control sequence.
(e) a heterologous nucleic acid that expresses the nucleotide sequence of any one of SEQ ID NO: 13-17 and 26-30 or a fragment thereof capable of inhibiting expression of SUV39H1 in the cell;
(f) a heterologous nucleic acid comprising the nucleotide sequence of SEQ ID NO:32-36 and 45-49 or a fragment thereof that expresses an RNA capable of inhibiting expression of SUV39H1 in the cell;
(g) a nucleic acid that expresses any one of SEQ ID NO: 13-17 and 26-30 or a fragment thereof capable of inhibiting expression of SUV39H1 in the cell, said nucleic acid operatively linked to a heterologous expression control sequence (e.g., promoter, enhancer, other regulatory sequence); or
(d) a nucleic acid operatively linked to a heterologous expression control sequence comprising the nucleotide sequence of SEQ ID NO:32-36 and 45-49 or a fragment thereof that expresses an RNA capable of inhibiting expression of SUV39H1 in the cell, said nucleic acid operatively linked to a heterologous expression control sequence.

2. The cell of claim 1 wherein the first nucleic acid comprises the nucleotide sequence of any of SEQ ID NO: 5-9, 13-17 and 26-30 or a fragment thereof.

3. The cell of claim 1 wherein the first nucleic acid expresses an RNA comprising the nucleobase sequence of [SEQ ID NO: 2] (lncRNA AF196970.3 exon 1) or any one of SEQ ID NO:13-17 and 26-30 or a fragment thereof capable of inhibiting expression of SUV39H1 in the cell.

4. The cell of claim 1 or 3 wherein the first nucleic acid expresses an RNA comprising the nucleobase sequence of [SEQ ID NO:3] (lncRNA AF196970.3 exon 2) or a fragment thereof capable of inhibiting expression of SUV39H1 in the cell.

5. The cell of claim 1 or 3 or 4 wherein the first nucleic acid expresses an RNA comprising the nucleobase sequence of [SEQ ID NO: 4] (lncRNA AF196970.3 exon 3) or a fragment thereof capable of inhibiting expression of SUV39H1 in the cell.

6. The cell of any of claims 1-5 wherein the first nucleic acid expresses an RNA at least about 12-50, 50-100, 100-150, 150-200, 200-250, 250-300, 300-350, 350-400, 400-450, 450-500, 500-550, 550-600, 600-650, 650-700, 700-750, 750-800, 800-850, 850-900, 250-750, 500-750 or more bases in length.

7. A cell comprising:
(a) a heterologous polynucleotide comprising the nucleobase sequence of [SEQ ID NO: 1] (lncRNA AF196970.3) or the nucleotide sequence of any one of SEQ ID NO: 13-17 and 26-30 or a fragment thereof capable of inhibiting expression of SUV39H1 in the cell; or
(b) a heterologous polynucleotide comprising the nucleobase sequence of the RNA expressed by [SEQ ID NO: 5] (genomic sequence), or by any of SEQ ID NO:32-36 and 45-49 or the nucleobase sequence of a fragment of said RNA capable of inhibiting expression of SUV39H1 in the cell.

8. The cell of claim 7 wherein the polynucleotide is at least about 12-50, 50-100, 100-150, 150-200, 200-250, 250-300, 300-350, 350-400, 400-450, 450-500, 500-550, 550-600, 600-650, 650-700, 700-750, 750-800, 800-850, 850-900, 250-750, 500-750 or more bases in length.

9. The cell of any of the preceding claims wherein the cell is a modified immune cell.

10. The cell of any of the preceding claims wherein the cell is a T cell, a CD4+ T cell, a CD8+ T cell, a CD4+ and CD8+ T cell, a NK cell, a Treg cell, a Tm cell, a memory stem T cell (TSCM), a TCM cell, a TEM cell, a monocyte, a dendritic cell, or a macrophage, a T cell progenitor, an NK cell progenitor, a pluripotent stem cell, an induced pluripotent stem cell (iPSC), a hematopoietic stem cell (HSC), an adipose derived stem cell (ADSC), a pluripotent stem cell of myeloid or lymphoid lineage.

11. The cell of any of the preceding claims, further comprising one or more engineered receptors, two or more, or three or more.

12. The cell of any of the preceding claims, further comprising a second heterologous nucleic acid that expresses one or more engineered receptors.

13. The cell of claim 11 or 12 wherein the engineered receptor comprises:
a) an extracellular antigen-binding domain that specifically binds an antigen, optionally comprising an antibody heavy chain variable region and/or an antibody light chain variable region, and is optionally bispecific or trispecific;
b) a transmembrane domain, optionally comprising a fragment of transmembrane domain of alpha, beta or zeta chain of the T-cell receptor, CD28, CD3 epsilon, CD45, CD4, CD5, CD8, CD9, CD16, CD22, CD33, CD37, CD64, CD80, CD86, CD34, CD137, or CD154, NKG2D, OX40, ICOS, 2B4, DAP10, DAP12, CD40; and
c) optionally one or more co-stimulatory domains from 4-1BB, CD28, ICOS, OX40, DAP10 or DAP12, 2B4, CD40, FCER1G;
d) an intracellular signaling domain comprising an intracellular signaling domain from CD3zeta, FcR gamma, FcR beta, CD3 gamma, CD3 delta, CD3 epsilon, CDS, CD22, CD79a, CD79b, or CD66d, 2B4, or any fragment thereof.

14. The cell of claim 13 wherein the engineered receptor is a chimeric antigen receptor (CAR) comprising:
a) an extracellular antigen-binding domain, optionally an scFv,
b) a transmembrane domain, optionally from CD28, CD8 or CD3-zeta,
c) one or more co-stimulatory domains, optionally from 4-1 BB, CD28, ICOS, OX40 or DAP10, and
d) an intracellular signaling domain from CD3zeta, optionally in which ITAM2 and ITAM3 have been inactivated.

15. The cell of claim 13 wherein the engineered receptor is a modified TCR comprising:
a) a first antigen-binding chain comprising an antigen-binding fragment of a heavy chain variable region (VH) of an antibody; and
b) a second antigen-binding chain comprising an antigen-binding fragment of a light chain variable region (VL) of an antibody;
wherein the first and second antigen-binding chains each comprise a TRAC polypeptide or a TRBC polypeptide, optionally wherein at least one of the TRAC polypeptide and the TRBC polypeptide is endogenous, and optionally wherein one or both of the endogenous TRAC and TRBC polypeptides is inactivated.

16. The cell of claim 13 wherein the engineered receptor is a chimeric co-stimulatory receptor that comprises (a) an extracellular domain of a co-stimulatory ligand, optionally from CD80, (b) a transmembrane domain, optionally from CD80, and (c) an intracellular domain of a co-stimulatory molecule, optionally CD28, 4-1BB, OX40, ICOS, DAP10, CD27, CD40, NKGD2, or CD2, preferably 4-1BB.

17. The cell of any of claims 13-15 wherein the extracellular antigen-binding domain binds an antigen with a KD affinity of about 1×10⁻⁷ or less, about 5×10⁻⁸ or less, about 1×10⁻⁸ or less, about 5×10⁻⁹ or less, about 1×10⁻⁹ or less, about 5×10⁻¹⁰ or less, about 1×10⁻¹⁰ or less, about 5×10⁻¹¹ or less, about 1×10⁻¹¹ or less, about 5×10⁻¹² or less, or about 1×10⁻¹² or less.

18. The cell of any of claims 13-15 wherein the antigen has a low density on the cell surface, of less than about 10,000, or less than about 5,000, or less than about 2,000 molecules per cell.

19. The cell of any of claims 13-18 wherein the extracellular antigen-binding domain binds to orphan tyrosine kinase receptor ROR1, tEGFR, Her2, p95HER2, LI-CAM, CD19, CD20, CD22, mesothelin, CEA, Claudin 18.2, hepatitis B surface antigen, anti-folate receptor, CD23, CD24, CD30, CD33, CD38, CD44, CD70, EGFR, EGP-2, EGP-4, EPHa2, ErbB2, 3, or 4, FBP, FcRH5, fetal acetylcholine e receptor, GD2, GD3, HMW-MAA, IL-22R-alpha, IL-13R-alpha2, kdr, kappa light chain, BCMA, Lewis Y, MAGE-A1, mesothelin, MUC1, MUC16, PSCA, NKG2D Ligands, NY-ESO-1, MART-1, gp100, oncofetal antigen, TAG72, VEGF-R2, carcinoembryonic antigen (CEA), prostate specific antigen (PSMA), estrogen receptor, progesterone receptor, ephrinB2, CD 123, CS-1, c-Met, GD-2, MAGE A3, CE7, or Wilms Tumor 1 (WT-1), or optionally the extracellular antigen-binding domain binds to any of the tumor neoantigenic peptides disclosed in Int'l Pat. Pub. No. WO 2021/043804.

20. The cell of any of the preceding claims that comprises two engineered antigen receptors that each bind different antigens.

21. The cell of any of the preceding claims that is autologous or allogeneic.

22. The cell of any of the preceding claims, wherein SUV39H1 expression is reduced or inhibited by at least about 50%, 60%, 70%, 75%, 80%, 85%, 90% or 95%.

23. The cell of any of the preceding claims where the cell is isolated from a subject suffering from a cancer or at risk of suffering from a cancer.

24. A modified oligonucleotide comprising a nucleobase sequence from any of [SEQ ID NO: 1-4] at least about 12 bases in length, wherein the modified oligonucleotide comprises one or more of a modified backbone linkage, a modified sugar moiety, a modified phosphate moiety, a modified nucleobase, or a chemically conjugated moiety.

25. The modified oligonucleotide of claim 24 that is at least about 12-50, 50-75 or 50-100 bases in length.

26. The modified oligonucleotide of claims 24-25, wherein the modified backbone linkage comprises phosphorothioate, phosphonoacetate, thiophosphonoacetate, methylphosphonate, boranophosphate, or phosphorodithioate.

27. The modified oligonucleotide of claims 24-26, wherein the modified sugar is modified to replace the 2′ OH-group by another group optionally, H, —OR, —R (wherein R can be, such as, alkyl, cycloalkyl, aryl, aralkyl, heteroaryl or sugar), halo, —F, —Br, —Cl or —I, —SH, —SR (wherein R can be, such as, alkyl, cycloalkyl, aryl, aralkyl, heteroaryl or sugar), -arabino, F-arabino, amino (wherein amino can be, such as, NH2; alkylamino, dialkylamino, heterocyclyl, arylamino, diarylamino, heteroarylamino, diheteroarylamino, or amino acid); or cyano (—CN); optionally 2′-O-methoxy, 2′-O-methoxyethyl modifications, 2′-fluoro, 2′-deoxy, or combinations thereof.

28. The modified oligonucleotide of claims 24-27, wherein the modified nucleobase comprises one or more 5-methylcytosines, modified uridines, such as, 5-(2-amino) propyl uridine, and 5-bromo uridine, modified adenosines and guanosines, such as, with modifications at the 8-position, such as, 8-bromo guanosine, a deaza nucleotide, such as, 7-deaza-adenosine, or O- and N-alkylated nucleotides, such as, N6-methyl adenosine, or a modified nucleotide which is multicyclic (such as, tricyclo; and “unlocked” forms, such as glycol nucleic acid (GNA) (such as, R-GNA or S-GNA, where ribose is replaced by glycol units attached to phosphodiester bonds), or threose nucleic acid (TNA, where ribose is replaced with α-L-threofuranosyl-(3′→2′)).

29. A nucleic acid operatively linked to a heterologous expression control sequence, said nucleic acid comprising (a) a nucleotide sequence that encodes or expresses an RNA comprising the nucleobase sequence of [SEQ ID NO: 1] (lncRNA AF196970.3) or the nucleotide sequence of any one of SEQ ID 13-17 or 26-30 or a fragment thereof capable of inhibiting expression of SUV39H1 in the cell, or (b) the nucleotide sequence of [SEQ ID NO: 5] (genomic sequence encoding AF196970.3 (ENSG00000232828)) or any one of SEQ ID NO:32-36 and 45-49 or a fragment thereof that expresses an RNA capable of inhibiting expression of SUV39H1 in the cell.

30. The nucleic acid of claim 29 that comprises the nucleotide sequence of any of SEQ ID NO: 5-9, 13-17, 26-30, 32-36 and 45-49 or a fragment or fragments thereof.

31. The nucleic acid of any of claims 29-30 wherein the heterologous control sequence is a constitutive, inducible, or tissue-specific promoter, optionally EF1α, CMV, SFFV, hPGK, RPBSA, or CAG.

32. A vector comprising the nucleic acid of any of claims 29-30 and one or more additional expression control sequences.

33. The vector of claim 32 that is a viral vector, optionally an adenovirus, adeno-associated virus (AAV), poxvirus, papillomavirus, lentivirus, retrovirus, herpes virus, foamivirus, or Semliki Forest virus vector, and including pseudotyped viruses.

34. The nucleic acid or vector of any of claims 29-33 in a delivery vehicle, optionally a liposome, lipid-containing complex, nanoparticle, gold particle, or polymer complex.

35. A method of producing the cell of any of claims 1-23 comprising (a) introducing into the cell (i) a nucleic acid of any of claim 28-30 or 33 or (ii) a vector of any of claims 30-33, and optionally (b) introducing into the cell a nucleic acid encoding an antigen-specific receptor.

36. A method of producing the cell of any of claims 1-23 comprising introducing into the cell a heterologous expression control sequence in a manner such that it is operatively linked to an endogenous nucleic acid that expresses an RNA comprising the nucleobase sequence of [SEQ ID NO: 1] (lncRNA AF196970.3) or the nucleotide sequence of any one of SEQ ID NO:13-17 and 26-30 or a fragment thereof capable of inhibiting expression of SUV39H1 in the cell, optionally a nucleic acid comprising the nucleotide sequence of [SEQ ID NO: 5] (genomic sequence encoding AF196970.3 (ENSG00000232828) or any one of SEQ ID NO:32-36 and 45-49 or an allelic variant thereof.

37. A method of using a cell of any of claims 1-23 to treat a disease comprising administering to a subject in need thereof an amount of the cells effective to treat the disease, optionally cancer, an infectious disease, an autoimmune disease, an inflammatory disease, or an allergic disease, wherein the cells express one or more antigen-specific receptors that bind an antigen associated with a disease.

38. A method for treating a subject suffering from cancer comprising administering to said subject: (1) a cell of any of claims 1-23 or a modified oligonucleotide of any of claims 24-28 or a nucleic acid of any of claims 29-31 or a vector of any of claims 31-33, and (2) a second cancer therapeutic agent.

39. The method of claim 38, wherein the second cancer therapeutic agent is an immune checkpoint modulator, cancer vaccine, chemotherapeutic or anti-angiogen.

40. A method for treating a subject suffering from cancer comprising administering to said subject: (1) a cell of any of claims 1-23, wherein the cell is a T cell, NK cell, lymphoid progenitor or myeloid progenitor, comprising a genetically engineered antigen receptor wherein expression of the SUV39H1 gene is inhibited, and wherein inhibition of the SUV39H1 gene results in enhanced anti-cancer activity of said immune cell; and (2) an immune checkpoint modulator.

41. The method of claim 40 wherein the immune checkpoint modulator is an inhibitor of PD1, CTLA4, LAG3, BTLA, OX2R, TIM-3, TIGIT, LAIR-1, PGE2 receptor, EP2/4 adenosine receptor, or A2AR.

42. The method of claim 41 wherein the immune checkpoint modulator is an anti-PD-1 inhibitor or anti-PDL-1 inhibitor.


SEQUENCE LISTING (50 sequences)

SEQ ID NO: 1 - RNA, 925 nt, Homo sapiens
  misc_feature 1..925: note = AF196970.3
  source 1..925: mol_type = transcribed RNA
cgaggcagggctttggctactggagatcgtaggttcgaatcccgtctgggaagttcaacttgtgcacctgtaaaagaagctggcattattggcttgtactcaagggctggcacagagtgtgtcggggtgcggacgccccagccacgcccatcatccgtgcggaagatgcagaggtcatatcggatacccttctgtaccacacgatttgggcagtcatagccgcagcggcagcgggagttgcactcgtagatgggcagcccggctcgaagccgcacctggccctggtcattgtaggcaaacttgtgcagtgacgcccccgggcagcagcctccagtgggtgcccacagacagtcctggcactcgcagcccacagccacctggttgagggtgatgccctcaccaacacggtactcattgatgtacacgaaggcccgcggagggccgtccaggtccacctcattctctacagtgatgcgtcccagatggctgcgcttggcattgagctcctgctcccagcgacggagcgcccgcctctgcttggccttctgcaccaggtagttggccaagcttgggtccaggtgccggggggtctttgaccggtggtgccgccggagcagctccctttctaagtccttgtggaactgcttgaggatacgcacacacttgagattctgccgtggctcccaggtgctctctgagtctggatatccacgccatttcaccagggtcaaaggagaaaattccctttggaaacagatgtgggcagttggggacaagagggcaggacactaacttccttgtgacctgtcccctcccagagcatggtcaccccagactcacgcggatcttcttgtaatcgcacaggtactcgacttcaaagtcatagaggttcctcttagagataccgagggcagggcaggagagc

SEQ ID NO: 2 - RNA, 125 nt, Homo sapiens
  misc_feature 1..125: note = AF196970.3 exon 1
  source 1..125: mol_type = transcribed RNA
cgaggcagggctttggctactggagatcgtaggttcgaatcccgtctgggaagttcaacttgtgcacctgtaaaagaagctggcattattggcttgtactcaagggctggcacagagtgtgtcgg

SEQ ID NO: 3 - RNA, 600 nt, Homo sapiens
  misc_feature 1..600: note = AF196970.3 exon 2
  source 1..600: mol_type = transcribed RNA
ggtgcggacgccccagccacgcccatcatccgtgcggaagatgcagaggtcatatcggatacccttctgtaccacacgatttgggcagtcatagccgcagcggcagcgggagttgcactcgtagatgggcagcccggctcgaagccgcacctggccctggtcattgtaggcaaacttgtgcagtgacgcccccgggcagcagcctccagtgggtgcccacagacagtcctggcactcgcagcccacagccacctggttgagggtgatgccctcaccaacacggtactcattgatgtacacgaaggcccgcggagggccgtccaggtccacctcattctctacagtgatgcgtcccagatggctgcgcttggcattgagctcctgctcccagcgacggagcgcccgcctctgcttggccttctgcaccaggtagttggccaagcttgggtccaggtgccggggggtctttgaccggtggtgccgccggagcagctccctttctaagtccttgtggaactgcttgaggatacgcacacacttgagattctgccgtggctcccaggtgctctctgagtctggatatccacgccatttcaccag

SEQ ID NO: 4 - RNA, 200 nt, Homo sapiens
  misc_feature 1..200: note = AF196970.3 exon 3
  source 1..200: mol_type = transcribed RNA
ggtcaaaggagaaaattccctttggaaacagatgtgggcagttggggacaagagggcaggacactaacttccttgtgacctgtcccctcccagagcatggtcaccccagactcacgcggatcttcttgtaatcgcacaggtactcgacttcaaagtcatagaggttcctcttagagataccgagggcagggcaggagagc

SEQ ID NO: 5 - DNA, 38301 bp, Homo sapiens
  source 1..38301: mol_type = genomic DNA
tgctgtttcggcgcaagcgctttggctcctgggttggcgttcagggttgccgaggcagggctttggctactggagatcgtaggttcgaatcccgtctgggaagttcaacttgtgcacctgtaaaagaagctggcattattggcttgtactcaagggctggcacagagtgtgtcgggtaatcttttccattttcttttacctattctttctttttttaagcaatgcctattgcatcccattatattgatttcaggacccactcaccgttcacaacagcactaagaaaaattcttcagggtccctggcatttgtctttcaggctacctgtaggtctgagtccagcagataagattcaaggtgatctcatgatcctcctccaggccagcccccatctcccagaagagggtgatttggaggcttccctcaggaccaggcctgactaggagtgtgatggggtgggctataagtgattaaaagagagatgcatggtttcacgctctgggatgctgaagagcttgtggcaactacaaggagaattttggaaggtgggggcaagacttaaaagagtacgcttttaaatgatgggattttaaacatcagacaggggaggtgtttggctggtgaggtaggtaggtagtaggaagaaagtgattctcaagtcaagtcagctcatcatagggtagttattacactctgcagtttggaggactgtgtgcaggatgtgaagtctgatttgttttggaatctcagttcagggtatgacttataaaaagactgtgaagtctctttgctgtttgaaaatctgtgtctcagaggtttaggtgtttgagatctttgtgggtctctgtcccagggttggtttcttttttctttcttttttttttgtttttttttgtttttcttttctgtttttctgtttacgttggtccagaatgtcccaaggtttctaagctgtttatggatctttgtccccggatgtgcagagtttttttttcttttctttttttgtgtgtctttacttttatttatttatttttgagacaggatcttgctctgtcacccaggctggagtgcagtggtgtgatctcggctcactgcagcctcaacttgccaatctcaggtgattctctcacctcagcctcctgagtagctgggactacagacacgtgccaccacacccggctaactttctttattttttgtggaggtgggattttactatgttgcccaggcctttttgtatgtttttaggggcagggtctctctctgttgcccaggctggagtgcagtggtgcaatcatggctcacttcagcctcgacctctggctcaaacaatcctcccaccttagcctcctgagtagctgggaccacaggtgcgtaccaccacacttggcccggctattttttttttttcttgtaggctgggcacggtggctcacacctgtaatcccagcactttgggaggccaaagtgggtggatcccttgagcccaggagttcgagaccagcctggccaacatggtgaaaccctatctctacaaaaaaaaaaaaattagccgggtatggtgacacgtgtctgttgtccccagctactcaggaggtcaaggctgcagtgaactgtgatctcaccacagcactccagcctgggcaacagagcaagaccctgtcccaaaataattttttttgcgtgtggagacagggtctccctctgtttcctaggctggtctcaaactcctgggctcaaatgatactcctgactcagcctctcaaagtgttgggattacagatgtgagccaccacactcagcctttttttttttttcttaaaattgattttcatttttcttttattggcatttctgtgactgctagaagggcttaacattgttttctgtgcttgttggcaagatgtatctaagttttgttttgttttgttttgagacagggtctcactatgttgcccaggctgagtacagtggcatgcacatggctcactgcagcctcgacctcctgggctcaagcagtcctcccacctcagcctcctgatcttgagtagctgagaccacaggcatgtgccaccacactccactaatttttttattttttgtaaagaccacgtctcaccatgttgcccaggctggtcacgaactcctggactcctgtcttaccctcccaaagtgctgggattacaggcatgagccactgcacttggccttatttaagttttctaaattcccttttacgtcctgtgatcatttttgttaaaatttttattttattttattttttggacacagggtcttgctttgtcacccaggctggagtgtactggcgtgatcatggctcactgcagcatcgacctcctgggctcaagcaatcctcccacctcagcctcccaagtagctgggaatacaggtgtgtaccaccacccttggctaatttttgtatttttgtagagaaggggtttcaccatattgcccaggccggtctccaacttctgagctcaagcgatctacccacctcagcctctgaaagtgctgggattacagatgtgagccactgcgtccagcctttaacattttttaaattttaaaagcattttagacttaaaaaaattataaaaatagtgcagagagttcttatatactctttttctcctaatgcttacttatataatcttagtacaatttttaaaatcaggaaatgaatgacacggttatttatgagctgttgttcaccggtttgtgtccagggtgtgaaatatctgagctctttggacccagagctctcttgggttttgtgtctcatgatgtgatgtctctgaacatttggtatatgtttttagatgctaggtctttcgtcatcttggtagtctttgtcccagagtatgatgtctttgacctgttagtagtctctttaggcttgtcttctctgggctgtatgggcttccatgttcaaggtgtgaggtttctgagcactttgaaggactatgttcaaagatgtgtatactgtcagctggtttttattgtcttctatcagagataatgaggtcaataaaaacttttcggttctgtatccagggcagggttcctcagccttgggactgttgacattttggaccagaaaattctttgttatggagggctgttctgtgcattgtagggtctattgtatttgtttactagggctgctgtcatgacaaagtaccacaatctgggtaacttaaatagcagaaatgtattgtctcacaattctggaggctggaagtgtgagatcaagatgttggcagggttggttcctttggggcctatgaggggaatgtgtgccctgcatctttcctagcttctcatgcctgctggcaatctttgctgttccctggtgtctgctgcatcaccccatctctgctttcatctttaccttgcattctccctgtgtgtcttgtgtccaaatttccgctttttataaagacaccagtggcgcaggattaggggcccactctactccagcatgatctctaacttaactaattacatctgcaacaaccctatttccaaataaggtcacattctgaggtactggggtttaggacttcaacgtaggaattttgaggggacataatttcacccataagagacgtttagcatgagagaaaaagggagggaagaagggagcagcctctgacacccagaagcaggcccggcccccataggtaggccatgttattctcctaacggacgtgaacaatctcacagaataccagcgtcaggtaaggttagtctgagaccatgataaaatcgacagagcttggccggatgaggtggctcacgcctgtaatactagcactttggggggttgaggtgggccgatcgcttgagctcaggagttcgagaccagcctggacaacatggtgaaaccccgtctctacaaaaaatacaaaaattagtcgggcatggtggtgggtgcctgtaatcccagctacttgagggactgaggccagaggattgcttgaacccaggcatttgaggttgcagtgagccgagatcatgccacactgcactacagcctggggataaagtgagaacttgtctcaaaaaaataataaaataggccgggtgcagtggcttacgcctgtaatcccagcactttggcaggccgaggcgggcagatcacgaggtcaggagatcgagaccatcctggctaacacggtgaaaccccgcctctactaaaaatacaaaaaattagccgagcgtggtagcgggcacctgtagtcccagctactcgggaggctgaggcaggagaatggcgtgaacccgggaggcagagcttgcagtgagccaagatcgcgccactgcactccagcctgggtgacggagcgagactgtgtctcaaaataaaaataataataataataataataataaaataataaaatagagcacagccactttataatgttgtttaagcacaaacaaaaataaggattcactgtgttgcccataaaatactaaacattctcctctcttggttaaaatgagtgactgccacttccttaccaatgacagcgttagcctcactctagtctgccctccctatagataagatttattgaaatacccaattgtaaaattgcccttgttttctgacagcatctaatctagagtgagcccccacttccttagaccctacccccaaatcactcaatcaaacccgaccaccccacacttccccatggagtgcgttctccctcactgcaatgagtaatcatcccaacttgttccactacaggtatgttcctgggtggggcgggggtctttggctaaaggacactggcaagctgtatccctggcttcaactcacagatgacagtagcacgcccgcaacacatatttgtgacagccaaaatgtccttagatattgccaaatgttctttgcggggcagaatcgcctctggttgagaaccactggtctgtggggtatgaagcgtttgaacaatttaggggtctttgtctgaggtttatgttctctgagaggtgtttgggtctgtgtctcagagtgtgaggtctgtggtctgtattacggtttctgttccaaggtttgaaagctctgagctctttcggggtgctatatcaggaattagtgaccttttcacataccaaatccagacatgaggtatgtgtgacatctccttttccctctgcctggaccatatgccagttcagaaaggcagtgagggttgggtgtggtggctcacgcctataatcccagcatttgggaggccaaggtgggtggatcacttgaggtcaggagtttgagaccagcctggccaacatgacaaaaccccgtctctactaaaaatacaaaaattagccgggtatggtggtgcatgcctgtaatcccaactacttgggaggctgaggttgcagtgagccgagatcgcaccactgcactcctgggcgacagaccaagactctgtctccaaaataaagaaaaagaaaaagaaaggtggtgagaagtaggaaccccacaattggttgtgaactcctccagtacacattaggatcgggattcacagcaatcccagcctctttgtctagaccagcaggcttccctgggccacattggaagaataattgtcttgggccacacataaaatagctaacactaacgatagctgatgagctaaaaaaaaaaaatcgccaaaacatctcataatgttttaagaaagtttacgaatttgtgttgggccacattcaaagccgtcctgggctgcatgtggcccatgggccttgggttggacaagcttggcctagaccataaagtataaattaccaacttccttcagaacctatctcgccctctgttctggagggcacagtttcccttgacactgtttgtggagattgtgttgtagggagatgtttgttctgtgtctcctgacatcagccactgagtggggacccccatctctcaggatggacgttgctcagatggatgttggagtccatctccccttaggctttgatcacactgatgataacccagaacacatggactttggagcctgatagaccctctttcaaagcctggaacccccaagtcctggttggatggtaggagagtctagcgttttggatcatctaaaccttgagctgctgaggtttttacaagactggtaagaatcggtaggacaatgcacagtccacacacgctctcctgatttcagtcccttccccctaatcccacaccccttctgaaagtagaattcagggctgggggaagggaagcaagcaatccatataaatgccaggggccagtgtgggcttgggactgtctcagttggaacttaggaatcatgtgcacatgttgtttaatgtaaatgtatgttgggtttaagaaagagtaaacactccccagaattgtgtcttcatctctagtaaatccacagttttacttcagttgttctcttgagaacatgcataatagattgtgcaccatgaggcaaggatgcacagtgagaaaaaactttgttcctcttctgaaaactgtgcttatgagacgggtttatacatttatgtgagaggcagggtatgagctccagagaaagactttaggggaggatgcagggccccagtagctcaaagtagtctcaagagaaataaaaaatagtttggactaaatgaaaatcaaaataaaacttaccaaatttgtgggatgcagcaaagcagtacttacctagagggaaatttaaaaggcacttatggcttctgctgtactttttcttggatcactgactagtagaagccagccatcattcgaggatgctcaagcagccaaaagccggtaaatttgggggttattttttatgtggcaatagataactcataagaggtgtttctgagctctttgatggtctgtctggagttcgagactttggagctttttcgtggtctgtatccatgccatgatgtctgggtcttttgggcattctatgtttcgcatttttaagtgaccttttggggttctctctctaggtttttagatctctgaattatttgggaatctatgtcccaaagcatggggattctgggctgtttttttctgttcatgttcagaacgtgatcaagtccagaatttgaattatctgagctctctggcagtctgttctgggtatgaggtcactggggtgtttggcagtctgtgtcccaggttcacatccatgcactctttggaggacggtgtattgtttctgtttattctttgaggtatatggatttctaagtcccaggaagagacatcaccaaggtgactggattctgcatccagaacactaggtgtcagctgtttgggccactgagtccctggatatgaagtctttaatgtgtttgagaacctgtgtcccgactttgatatttctgagtttgagggcagagggaatcagtactctgcactgtttatgagtatatatctcaggatgtaaggctcccaaactgtttgtgagtatccctgacatttatgtcacagtatatgagatctctgttcatgggtgtatataccagggtgtaaggtatctgagccctttgatcgtgtgtgtccaaagttgtggggtctctaatctgtttgagtctatatttcccagaataagagatttctgatttttttgtgggtatatattccaagatgtgagttctgtgatccatttggaagtatatttgaggtttccgttctgtttgtgggtatatgtccaatggtgtaaggtttccaatgtgcttgtgagtattcatcccagggtatgaggcctctgatctgcttgtcattatacaatgacaagatgtgaggtctctcttcatgaatgcatgtcaagggtttgaggtctctgatctgtttttggttttatgtcccggaatgttagatctctgatctctttgtgggtgtatgtgccaaggtgtgagtctcagctttttgtggctatatttccctagatgttatatttctgagcttttctggttgtatttttttagggatgtcagtactttaatctgtttatttggtgtgtgttggagaatatgaggactattctgtctgagggtgtatgtcctagggcatgaggtctctgctatgactgtgggtgtacatctctgggtatgaggtctccaaaatgtttgtgggtatatttcccaggatgtgagttccctgagctcctagtgggtgcatgtaaggtctcagatctgtttatccctgtatatgttcctgtatatgtgaagtgtcaagggtatgaagtctctgatatttttgtggctatatgttccaggatatgaggtttctaatctgtttgggggtggattgctcagagtgagttctcaagtatgtttgtggttatatgagctaggatttaagtatctgatctttttgggtgtgtaagtcccatggtgtgaagtctctgagctctttgatggtatatatgcagaggtgtgagatgtctgatttgtttggtggtatatttcccaggatgtgaggtctctgatccatttgttgctgtatactagtatgtgattttcattggtgtatgtcaaagtcagtttgttggtgtatttctcaggatatgaggtctctggtctgtttctggatgtatgttcccaagtaagaagtctctgatccatgtgcccacatatgtctctgtatgtaaggtctctgatctgttcatggatacactgagtttgagatctttcatctggttgcaggacaggcaagccccaaagttgaggcttagctcaggagggttcttggctttgcctaggaaggaattcaagagtgagctggtggtgttaaatagcagcttttattgaagcggcagtgtatagcaggagcagatgtgctgctccttgcagagaagagctacccataggcagtatgcccaaagtagcagctcagaggccgttctgcagtcatatttatgcccaccttttagttacatgcaaattaagggatggtttatgcagaaattttaggtatctgggctatttctgcaagtatgtcccaggatgtgaggttgaggtctctgacatgtttgtagatgtatttactagaatagaagatctctgacatgtttgtggatgtatgtcccagtgtgtgaggtcactgatctgatctgtgtttgtgcatgtatgtcccaggattttaggtctgtgatgtgtttgtcattgtaattatcagtatgtgaagcatctttcatgggtatatgctaagaatagaagttctctgatatgattgtgattgtatgaacctaggtatgagttctcttatctgtttgtggagtatgtcccacagtgagaggttcctgagctggttgggggtttatttactaggatataaggcttatgacctttttcttgttgtgtgtgaggtccgtggctatcgttgtatcattgtgtatcctggtatgtgagatctttttcatgggtgcatgtaaaaaatgtaaggtctctgatctatttgtgattatatgtccctgcatattaggtctctgatcagtttgtggatttaattcctacagtttggcatcatcaatctgtttgcaggtgtatgtcccatgatgtaatgtctgatcttttcgaggatgtgtgaggtatttcatctattcctcagtgtgtttccaaacatgaggtctctcaactgtgtgacagtgtatgttctgtgatttgaggcccctattttttttttacaggtttattcccaaggatatgaactctcctgtctcttttgggtggtatgtcacaatttgagaggactgattcatttctgagttgatttcccagtatgtgaagtctctgatctgtttgtggtatatactgcagaatttaatttttatttatttatttatttatttaagatggcgtctcgctctgtcgcccaggctggagtgcagtggcgcgatctcgactcactgcagcctctgccttctgggttcaagcgattctcctgcctcagcctcctgtgtagctgggattacaagcatgcaccaccatacccggccaatttttgtattgttagtagagacgggcatgtttgtgcatgttgaccaggctggtcttgagcgcctgatctcaagtaatcttcctgcctcagcctcccttttaaaatgctgggattacaggtgtgagccaccgcacccagccttagttctttatgcatatacataccaagctttgaggactctcacatgtttctatatatgttctggatgtacctaatataatctgtttagttgtgaatgtccagcgtgtggggtttcaaagagcccgtagtgaatgtatatcttttggtctgaatcctctgatctctttgtgggtacatgtgggttcctgttcctggatatgagctctatgatctctttgtgggtgtatgtcacaggttatgagatctatgatctatttctgactttttttcatagcatgtctggtttctgatgtgcttgtaagtgtatgttgcagaatttaggatccctggtcatatatagtctctgatgtgttttggagtatatcccataatatgagctcccttatctgtttgttggtgtacatctcaagagtgtgaggtctctgatatctttttttttttttttgagacacagtcttgatctgtcacccaggctggagtgcagtggcacaatctcagctcactgcaaccgccacctcccggattctagtgattctcctgcctcagcctcccaagtagctgggactacaggtgcccgccaccatgcctggctaatttttgcatttttagtagaggtggggtttcaccatgttggccaggctgatcttcaactcctgacctcaaatgatccacccacctcggcctcccaaagtgctgggattacaggcgtgagccaccacgcccggccgtctctggtatctttatgggtatatgttgccgggtttgaggcctgtggtctctgtgtggttacgtgtctaagttatgaggtctctaatctgtttgctgatgtatatccctgatcttttttttttgtaggtgtatgcctaatggtgtgaagattctgatctgttttctggtaaatgaaccgggttgagataagtcagacatccttgaaggagttttttcagaatgcgagttccctaatgtttccgaatacatatcctaggatgtgatgtcccagggctatttgtggatatatatcccaggtgtgagttttctcagccatttatggatatacatcccaggttggtagatatcttaggcttttggcattatttacaagcatatgagatttctgatttgtatgctcatatacataacaggttgcaaggcgtattatttgttacagtgtatacatccagggtatgaggtttctgagctctttgttggtgtatgtctcagggtcgaggcttttgatctgtttgcaggtatatgtcccaggatcgtagctctctcatctctttgtgggtgtgtaccccaagatatcaagtatttttaattattctgagtgtgtatttgccaggatgtgagatcgctaacatgttgtgggttcttgttgcaggattttagacctgatccattttggttatatttctcagatgaagtctctgatctatttgtgtgtgtatgttacagtatgtgagtttaaaaaaaaaaacctcggctgggcatggcggctcacacctataatccgagcactttgggaggctgaggtagaaggatcacttgagcccaggagtttgagaccagcctgggcaacatagtgagacccccgtctgtacaaaaaataaaaaattagccaggcttggtggcgcatctgtagtcccagctattggggaggctaaggtgagaggatctgcctgagcctgggagttcgaggctgcagtgagtctttatcatgccactgcactccagcctgggtaacagagtgagaccctgtctgaaaaaaaaaaaaaagtctcttttgcttgcttatcaagggtatgaggtctgttctgtttgtgggtgtatgtcccaatttgtgagatcccagaactgtttgtgggtgtatgtcccagaacacgtgtgtgtctgctcgctctttagatgtatgtctcaaggtaggaggtgtgtgatcactttctagatgtataactcaggatgaggtaagtgagctgtttgtggtgtgtgtcccagcatttcaggtctctgagctttctggaagtgtgtgaagtctcccattgtttgtgcatgtatgtttttgttgtgttgacttaaggcccccgaagatgcccatgtcctaatctttggaaactgtgatgtgactttacatgctaaaaggtaccttgcaaacagcatggatggagctggaggccattatcctaagtaaaatgactgaaacagaaagtcaaaaactgcatgttctcacttataagtgggaactaaacagtgggtacacatggatatacagagtagaataatagacaccggagactccaaaagatgtgagggtgggaaaggggtgagggatgaaatactgcctattgtatacagagtacattattccggtggtaggtacactaaaagcccagacttcaccactacacaagatagccatgtaacataactgtacttgtacacctaaatctataaaaatttatatctaaatatataaaaatttaagtgtttgcttcagcagcacatatactaaaattagaatgatacacagaagattagtatggcgcctgtgcaaggatgacatgcaaattcgtgaagtgttccagaaaatttttttaagattaaattattttaaaaatgtttattaaaaacatttttgaaaggccaggcgcagtggctcatgcctgtattcccagcacttaggaaggctgaggtgggcggatcacttgaggtcaggagttcgagaccagcctggccaacatggtgaaaccctgtctctacaaaaaatacaaaaattagatgggcatggtggtgcacgcctataatcccagccactcgggaggctgaagcaggagagagaatcgcttgaacccgggaagtggaggttgcagtgagcagagatggtgccactgcactccaacctgggtgacagagtgagactccatctcaaaaaaaaaaattttttttaaaaggtaccttgcagatgtgattaaattaagcatcttgagatcgggagattttcttggattttcactgaatataatgacacagactgagtcagagacagcgatatgatgacacaagtgttgggggtgtgggtcccagctcatgaagtccttgcgctgttcatgggagtatatcccaggaggtgaaatctctaattgtttgtgcacaccagtctgtggttggctgaataaagcatccctcaaagatgtccagtgaaagagatatgaccagggagccctaaatgatggaagggaatgtgatggtggaagcggagtcaaaaaggagatgccacaacagaggtagacatcagagagcattttgaagatgaactgctgtccgctgtgaagatggagaaagggggaagggatgaaggactgcaggcagctctagaacccagaaaaggcaaagaaacgtgctcttccttagagtctccagaaagaagacagctctgttgacacctgactttagtaccctaaggcccattcttgggtttctgccctacacagatgtaatataataaataaatttgtgtttttcacagcactaagattgtggtgatttgttacaacaataagaaatgaatactgtgtcccaggtgcatgatgtctctgtctggttgttagtgcaagatgtgaatccccaatctgtttgtgagtgcatgtcttttatttatttacttatttatttatttttgagatggagtcttgctctgtcgcccaggttggagtgcagtggcacgatatcggctcactgcaacctccgcctcctgggttcaagtgattcttctgcctcagcttccccagtagctgggattacaggcatgtgccaccacgcctggctaatttttgtatttttagtagaggacagggtttcaccatgttgatcaggctggtctcgaactcctgacctcaggtgatccacctgcctcggcctcccaaagtgctgagattacaggtgtgaggcaccacgccccgccgagtgcgtgtcttatgtggcagatctttcatctgtgtgtgggtgtatgaaccagggaggaggtctctgacctgtctgagggtgtctatccctcagtttgtgttctctgaggtttctgcaggtagtctcaggatatgaggtctctgacctgtttgtgggtgaatgtcctagaatgtgaggtctcttggctgatttggagtgtatgttccagtaagtgagatctctgaggtaataataatatatataataacctatataatatataatgtatatgttcacaatatttataattgtgcattataattattatatgatattatatcatatcaatatattaacatataagcatatgttaagtatatattatatatacataatagtatatataatatgttaacatattaacatgaacagataactatatagtatatattatatttatatataatataccataatatattatatattgtattatattgtgtagtaacaatatatattatattatggtatatttcacaatataccattcacaataaatattgtgactacattatgcatggcttgaggtctctgatatttttgtgggtttatatcccaaggatatgaactctgaattctctgtgggtgtattctgaagatgtcaggtgtatactcagtctctggctgtttgttctagaatgtgagatctcttctttgattgtgggtgtatgttacaggttgagatctctgttctgtttgtggatgtcttggagtgtgaggtctctgatctgtttgtgagtaatgtattgcaggaattgtggtctctcatctccttgtgggtgtatttctcagatgaggcctccattctgcttttgcctttatattgcagtatttaagtttctaatctcctcatgtgtgtttgtcaagggtgtgaagtatcttctgtttgtgattatatgtcccagtgtgtgaagtccccaccctctttgtgggtatatgacacagaacatgtgcttcacttctttcttggatgcatgtccaaaggtgggaggaggtctctgatccctttggaattatataacataggaggtggagtctttgagttgcttatgtgtggatacctcagcaaatgatgtttttcattgcttgtagatgtatatcttaggttggtggagtgaaggttcccatccttatatccctggaatgtgtgactgtgaccttacatgttaaagggtccttgcaaatgtgattaagtaatcatcttgagatgcggggacggttctggatttcccaggtgagcccagtgtaatcacaagggtccttagaggagagaggctggacagtccgagtcagagaagcagatgtgatgccagaagcagatatcagagagatagagatttgaagatgaaatgctcctagctttgaaggtggaggaaggggccacaagccaaggaacgcaagcagctctagaaactggaaaaggcaagggcacagattctcccctagagcctccaggagtaatgcagctctgctgacactgtgattttagccctttgaaacccatttttttcttctgacctgcagaaaagtaagataataaatgtgtgttgttttatgacattatgattacgatttgttacacattgatctctgtttattggtacacatcccaggatgtgaactccatgatctgtttgtgagtgtatgtctgatggtggaagtctctgatctgtgtgcagttgtatgaaccacattgagaggtctctgaatgttgagggtgtgtgtcccagaatccgtggtctctgagctgtttgtgtgtgcatgtactaggatgtgacatctttgagctgatttttgggagtacattccagtaggtgaagtctttgagatatttaaggattatgttccaggttgttacgtctgtcatctgtttggggggtatttcctgtgacatgaggtttctgatttgtttggttacatatgtaccaggatgttaggttgttaggtgtctggtctgtttaggtttatttgtctagggtgtgaggtttctgagctctttgtggatgtatgtctcaggattgaggccactgatctgcttactggtaaagaaatgtcccagtccatgagctctctgatctgctcgtggttctgtgttatgagatgtcgggtctatttctggtgttttcctaacctgtgaggtctctgacttcattggagttgtacattgaaggatttgagatctctgtttgtgggtgtgtgtctcaggagtgtatggtctgtgcattgcttgtggagtatatcccaggatgtgaactttctgatcagtttgttatatatgactcagggcctgaggtttctgatctgtttgtggattgtgttgcagagtttgatgtctgtggtctctttgtgggttcatgtacaggttgtaaggcctctactctgtttgtgggtgcatatcccaggatttcagctccctgacctgtttgatgctgaatggcacataatctgagggctctgcgctatttttggtatttggggtctctgatctgtttgtggttacgtgtccatgttgtgaggtctgttatctatttgtcagtatatgtgccaggatgtgaactccctgatatacacgtaggggtgtatctgatgtgaattctgtgatctgtttgcaggagaatgaaccagggtggaaggtctttaacctctttgagggtatgtgtcccagaatatgaattctctggtctatttctggattcatgtcccaggatgagtgatctctgatctagttgagtatgtgtgtccagggaagaaagtttgtgaaccctttgtggtttcaggtcccgtggcctgaagcctctgatgttcgtggatacatgtccaggttgttatgtctctgatctgttggtatatgtctcaggatgttaattccctgatctgtttgtgggtggtgtctgatggtgtgaaatgtctgatgtgttcactggtgaatgaaccactgtgtgaagtctttgacctttgttgagggttttaacataggatctgaattttctgatctatttgtaaaagcatgccctatgagatgtttgatctttgagctctttatggatgtacatcccaggatgtgagttctctgagctgttggggcatacatgttctaatgttctaggttatcagacctctgaagtttttttttagttatatttccaagcatatgtgatttatatgctgacatatatattcatatatatatgtaacaatatagctgtccctcagtatctgtgggttggaggaattggttccaggacccctgtggatactaaaatccgtgaacactcaagtcccttatataaaatggtatggtatttgcatgtaacctatgtaaattcttctgtatactttagataatctcaagatttcttataatacctgatacaatgctacgtaagtagttgttatactacattgtttttatttgtattatttttactatgtttttaaattatttttttcctcagtgtttttgatccagacttggttgaatccgagggctgatagtagatataacatatatatatattgtgtgtgtgtgtgtgtgtataatatatatacctgatttgttgaggtgtgtatgtgtccaagctgtgaagttattgagctatttgtgggttatttcccttggtttgaaaacctctgacctatttgggagtatgtctcagaatataagctctctgatctctttatgggtgtatgttgcaagatttcaggtctgtgatctatttctggctcttttccctagcatgtgaggtctatggcttgattatgggtctgtattgcaggattgagacctctgatttgtttatgggtatctagctcaggttgtgaggtctctgatctgtttgtggctttatgtctcaagatgtcaggtctgtatacctattctgcatatatatcctgcaggtaaggtctctaggctgtttgtagtttcatgttgcaggattcaaggtctctgatctctttttggttgtgtttcttacatgaggtctgtgatctgtttgctagtgaatgtcacagtatgtgagtgtctcttttgtgtgtatttgtcaaggatgtgaggtctaattcaacatgtcccggattgtaagatacctaacctggtagtgggtttatgtctgagaatgtttctttctgcagtgtgtgtgtgtgtgtgtgtgtgtgtgtgtgtgtgtgtgtgtgtgtgtatgtcccaggtttggagatttgatctgttgggggtgcattttgcagcatgtggggcccctgatatcttcgtgggtatatatcctatcatgtcaggtccatgttttctttgagtgtgtatgtcccagatggtgagttctcttatgtgtttgtgagtttaattccaagtgattgaagactctgagtattttttggtatatgtcccagaatgtgtctctgctctttttggatgtctgttagagagtggaggtgtcttatctatttggaagtgtatgactcaggatgtaaagtctgtgaattgtttgtgtgtgtatgtaggtctctggtagctaatggtaccccaaagatgtgcacttcctaatccctgaaacctacaattatgctaatttacatggaaagggggactttgcagaggtgattgtgagggactttgcagaggtgactttgtggaagttaagcatcctgagatggggatgcttatcctagattaactgggcaggcccaatgtaatcacaggtgtccttatatgagggaggcgtgaaggcaagagtcagagagggctatgtagtgatggaagtacagaaaagatgatggacaacatatacagagatcaaagagagctagagatagatggatacaaggagagacagatgggggaggaggaagaaaggggggtggggagagagacagagagagagagaaagatttgaaggtgaaacagacctctggctttgaagatggaggaagaggccatgagctcagaaatgtaggcagcctacagagactggattctttggccaggcgtggtggcccacgcctgtaatcccagtactttaggaggccaaggtgggtggatcacgaggtcaggagatcaagaccatcctggccgacatggtgaaaacctgtctctactaaaaatacaaaaattagctgggtgtggtggcgcgtgcctgtaatcccagctactcgggaggctgaggcagtagaatcgcttgaatcagggagtcggaggttgcagtgagccaagatcgcgccactgcactccagcctggcgacagagcaagactctgtctcaaaaaaaagaaagaaagaagaaaaagaaaaaagaaattggattctccccaaggaactccagaaagaagtcagctctgccaacaccttgacttttaccctgtaagactctttgtcagaaatgtaagatcataagtttttttttatttttatttttttgagacagagtctcactctgtcacctaggttggagtgcagtggtgccatcttggctcactgcaacctctgcctcccaggctcaagcgattttcgtacctcagcctcccgagtagctgggactacaggcgcccgccaccactcccggcttatttttgtatttttagtagagacgaggttttgccatgttggccaggctggtctcaaactcctcggctcaagggatccacctacctcagcctcccaaaaggctgggattacaggtatgagccacccccagctgagtgagataataaatttgcattgttttatgacaaaaagcttttagtgatttgtttcagcagcaatagaaaatcaatagtgtgtcaggttgtgctgtctctatctggttgtcaggatgtgaacttcatgatctatttgtgagggtgtgtgtctgatagtggaggtcactgatttctgtgcagggtctctgatctgatgacctatttggtggtgtgtgtcccagaatttgtgctctcagcttttgtggaggagcgtatgttctaggatgggacatctccaatctgattttgtgtacatgtgccagtgtgtgagttctctgatctgttgaggtgcgtatgtctagggtgtaaggattccaagctctgtgtgagtatatttccttttgcttgaaattcttgccctgtttctgggtttatgtcccaagatgtgagactccctgatttgtctgtctctgtgtcatggtgtgaagcctctggtatgtctgctggtgaatgaagcagagagtgaggtctctcacctctttttttttgtttgtttttgagatggagtctcactctgttgcccaggctggagtgcagtggtgcgatcttggctcactgcaactgctgccgcccaggttcaagcaattctcctgcctcagcctcccaagtagctgggaccacaggcccgtgctactatgcctggataatttttgtatttttagtagagatgaggttttgccatgttgaccaggctggtctcgaactcctgacctcaggtgatccgcccaccttggcctcccaaagtgctgggattataggcgtgagctaccgcgcccagcctctcacctctttgagagggttctctcaggaagtgagttctctgatctgattctgaatgcttagcctaggatatgaggtctttgatctgtttgtgggtatatatccctggcatgaattttctgagctgttttgccaatatttgtgggtacttatgatatgcatataaagtttctggtttgtgtgctggtatttatagtagaagtcaggttttgttgtggtacatgtatccacaatgtggttttggagcactctgtgggagacctgtgatctcctcttgggtctataccaaagacagcaggtctctgtacttcttcaggagtattttctgcatatgaggcctgtgacctggtgtttcatgtcgtggtattagaggtctctggtatggtttctggttgtgtttctcagataggttctcagataggtctctgatccatttgttcgtgtgtgtcatcgtatgtgagtgtgtcatctcctcagggtgtttatagagggtgtgagtcccattctttttgtggttttatgtccagtttgtgaggttcctgacctgtctgtgggtgtctgtcaatgaatgtgtttttttgtgttctttgtgtctgtgtctcagagtcagagatctggtatgttcgagggtgtttgtcccagaatgtggggtccctgatctcttcgctagtgtatatcccagcgtgtcaggttccggttctctttgagaatgtacttggcagagtgtggttctcttatgtctttgtgggtttaatccccagggtgtgaggactatgagcatcatttggcgtatgccccagagtgtgtggctctctgatctttttggattgatgtccaggagtgggagttctccgatccctttggaggtctatgactccagctgtgagatctgtgagcagttcatgggtgaaagtcccgggatgtgagggttctctttgtgcatgtatgttctgtttcgtcagataatagatccccaaatatgtcctcacctcattcctggagcttgttaatatgttgtcttatctggaaagggggctttgcaaatacatattaggtaagcatcctgagatgggggcagttatcctggacaatccaggtgggcccactgtcatcacaagagtctttatatgaggaaggtaggaccttcagggtctgagaagactgtgtgtgtcacatcagagaggaccatgtgacaacagaagtagagtcacagaaggtgatgcgatgacagaagcagaggtcagagagaaatttgaagccaaaactgttgaatcatctgatctcttgggagttatgctgtaagatgtcatgtctaccatctgtttctgggtgtgtttttcagcacatgaagtgtgactggattatggttgtatactgcaggatttgaagtctctgattgtttttgtatgctccaggatgtgagagctctgagccatttgtccatgtacgttgctggagtggaggtctctgatctgtgtgggtgcatgtccacgttgtaaggtctctaatccttttgtcagtgtatatcccatgatgtgagccctctgatctgtttgcagctatattcgtaagatagttatctgtatgtttctgggtatgcgtgtgtgtcggggtctgttatctgtattctctttatgggtgtgacctcccctagatgtgaggtctctgatttgtttgtcattgtatgttccaggatgcaagatttgtgaatggtttgtgggtgtgttttgcaggattttatgtctgtgatctatttgaggttgtgtgtcataggatgtgaggtttctgatctttttctgggcaaatttcctagcatgcgaggtctgtgaacattttgtcagtgtcccaggctatgttatctctgatctcttcacatgtattgccaagtgtttaatgtctctgatttgtttcttgctgtttgttccaggacgtgaaatctcatactgtctgtggatatgtgtcccagggtgtgtggtctctgatctgtttgtgggtttgtatatcccagtgtttcattactgtctgtggatttatttcccaggattttttagtgtatttcctaggatgtgcatttctctggtctttctcaatgtatatcccacagtggacagtatctgttccctttgtgggtgtatatcccttggtttatgatctctgatcctttgtgggtttatattttaggatgtgaagtctctattgtgactgtgggtgtgtgtcccaagacgtaaattctctggcctctttttgggtttatgttccaggatgacaggctctatgtgtatttctagcctgcaaggtctattacttatttgtgggtgtaagtccaagggtatgaattctctgatctctttgagggtcagcgtaccagaatgtcaagtctagtttggttatgggtgtatgtcaagatgtgagctctctgatctccttgtggttgtatgtctcaggatgtgagggttctgatctgttcctgtatgtttgtcaacagtgtgtaatctctgatctgtttgtggctctgtgtcccaggttgtgaggttctgacctgtctttctttctttgtcccagcatgcgagatctctgaggtgtttgggtttatgtcccaggaattgagtctctggtctctttgtgagtgtgtgcctcaagtaatatgtgaggtccctgatgtagtctttggtttatttcccaaggtgtgagtgctctgatctattcatgggtatatgtcctggcatgtgaagtgtctggtatttttatgtgtatatgtctcagggactgagttctgtgatctgtttgtgggtataaggccccagatgttagttctctgaaatatttgtgggtgtatatctcagcttgtgtggtttttgatctttctgtgagtatgtcacaggatgtgaggtccccgttctttttgggtgtgtgtttcctagaatttggattttctgagctgtgtgtgtgtgtgtgtgtgtgtgtgcgcgcgcgcgcgcacatgtcacagggttgaggtccctggtctgtttgttggtgtatgtccaggatgtgagttctctgatctctttgggtgtgtacgtcccacaatttgaggcctctgaattagttgatactctgcatccagcatattaattctctgaaatatattttggggtctataccctaagtctcagaaggattctgaatcctgaatgcgagggttctgcgctttttctggtctacattccaggggggtgagttcactgggctgttttgcggactgtgtttaccttgtgaggtctctgagctgtttgtccactggtgtcctaggatacgtggtccctgagctcttttggtttcaaagtcccttgacctgtttgggatatattttcaggcgtgaagtctctgttccttttcagagggctcagtcccggggtttgatatatctgactcattaggggtttgttccagggttaaggtctctgagcttgtaatgggtgtgtgtgcgccagtgagtgagttttctgagttgtttgaaggtctataggtgtgaaatttcttaactctctggtggttcctgtgcagagtatgatgtctctgaactatttagcagtctctcttttcaggtgtgaggtgtttgtacccttgtatgagaactctgatgtatttgagtatcaccgccaaactgttttgagccaatgtcccaacaaatgagttatctgtgatgccgtgggttttgtcctagggttgattttcctgagcagtttcaggatctgtttccagagtctgatgtctttgtactaataatgtctttttactgtttcaagcctctggagtgcttcaccctctgtgtcccaaagtgtcagatctctgatctctgtattccttggtgttgaggtccctcaactgttatgtttccaagagtgagacagttcctgttgagggagtctcagtcccagggtatgaggtctgtgacttattaggggtctggttctagggtttggggtctctgagcagtttggggttctgttatcagtttctgacgtctctctagtatttgggggtctatgcccgggttgtatgtcactgggtcactcggctgttaaggggtctgtatcctagggtgtaaagtttgtcaactctgagcatttttgcattgtctgtctgctgtgtgaggtctttggcatgtttgtgggtttacttttcaggttgtgaggttaactctttgagggtttgtgtcccataatatgaggtgcttgagctgtgcgggcatacttagccaggatgtgatatctctgaactgttttcatgtctgtctcccaggttagaagttctctgagtcatttgggagcctgtgtccacaatgtgaggtcctaagccgttttgggtctgtgttccagttatgaagtgtctctactgtttgtggccaactttgcacaaaggtttcaaagctgtttggtgatctgtgtccatgactatcagccatctgaaatctttccggataaggacaaagggcaagttccatgaatggttttgaggactgagtccaggatgtgatattccttaagcatttagaggtctgcgtttagagtgggaagactccgaactgtttgggggtctgagcctaggatatgaggtttctaagctgtttggggtccgtgtccagggggtgtgatctcagttcctcagggtttttgtcatagggtgtgaagtctttaagctctatagagggtttatgtccacactttcaggtctgtggggcctctgagctgtttggggatctgtctcggtgtataaaggctgtgagctattttggggaactctatgtccagtatatgacatctctgaacaggaatctgtgtccagggtgtgagcctctgagtagttttcgggcctgtgttccagcataagtgattttagggctctctagcattgtgtccgtggtgcgaggtctctgggctgtctggtggtctgagaagaggttgtgaggtctctgaactgttggtccatttttgtcccagtttgtgagttctctgagaaattggtggatctttatccctgattgtgaagtctctgagtttcccggaggtctgtaatcagatttccacgtctctgagctctttgggtgtggggttacaaagtgtgaggtctctgaagtgtttggtattgtctgttatcaatagaatagaaatatattgattactaggctgccttggctttgaactgttgaggggtctgtgtcagaaggcgtgaggtcatgtttgagctgtaaacagatttgtttcccaggtatgagtcctgtgagcagggtctgtttccaggctgtgagatctctaagctcttggggtctgcttcaaagacttgagtctaagttgtttgtggcactatattcacgtgtaaggtctctgggatgtttacttgtctgttttctagggtgtctctgagcaatctgtgggtccctatacaaggtatgagttttcagaaccatttaaggatctgttttcaaagggcgaggtatctcgggtgtttgggcagtctgtgtccagggtgtgccagctcagttgtttgggggtctgtcccaggatactaagtctctgagcggtttggaagtcaatgatcagggtgtggagcctccaaatagtttgggtagtgacatctcaagttgtaaggtctttgagatggttgtgcatctctgtcatcaaaggaaaggtctctgatgtgtttaaggtctgaatcccagtatatgaggcagtatgtgaggtctaagagctgtttaagggtctgtagcccatagttttcagtgaagcctgtgttacttgcggtgtgtccaattatgagagctctgagatatatgtaggtgtgtatcccagggtatgaggtttctgagaagtttggggtcaatattcagggtatatggtctataaggccttataccctagatgccctgtgtatgtcctggttaccctgtgtatgaggtctctgggctttttgggatcctgccacctctgtatgaggtctttgaaatatttgggtatctgtcacggcacgtgaggtccctgagcaaaaacaaagagcactggctccctttcagattgagagatgatgaaaatgagtaagtatatctgaaatgtggtcttcattctccactttataccctggcaccatataggtgctcaataaatatttgcagaaaaagtttccccagcacacacaaacttttcattttgaatgctggaggcagattttgggttcaaagcctttaagagcagggaaggtccagctagaatattatcacattttaacctttccattgatgtattcctctggttatttttgtttgcttatggcaagtgattatggtttttccatttagacagcagtggatttggttttctccctccccactttgctccactccttctaccctcccccccacccccccagtgctgtatagtccctgaggcctgggcacttggtcaggcaggaggtggcagggtctcagccagacattaccagtcatggctggtcaccaccaatcatggccagtcaagactggtcaaagctggttatggctagtctaagttggtcaaagctgatcacggccagtcaaggccagttaccagagatcatggctggtcaaggccagaaaccactggtcatggctggtcaggatgggtcaaagccagtcatggccagtccaaactaatcaattatggtcacagccagtcacagctggtccaggttagtcacagcaggccaagaccagtcaaggctggtcaaagctggtcaaaaccaatccatttcagtccctgctagtccaggccaatccaagccagtccaggctgatcacagccagtttaagcagtcaaaactggtcaatcccagtccaagccagtcctgattggtcaaaacgagtcgaggccagtcacggctagctaccaccagtcacagccagtcatgacaagtcaaagtcggccaacacaggtcaagatcggtcaagtccagtcaaaaccagttacggccagtctaaaccagtcaatttaattgcattgttttgagacagggtttggctcttttctggagacagacaggctggagtgcagtggtgagatctcagctcactgcaacctccacctcccaggctcaagcaatcttcccacctcaggctcctgagtagctgggactacaggtacaggcacatgcccccacacctggccaatttttgtatttttagcagagacgtggtttcaccatgttgcccatgctggtctcgaattcctgagctcaagcgatctgcctgcctcggcctcccaaagtgctgggattaaagatgtgagccaccacacccagccttaagtgcattttaaagtttaagtataaaacaaacttattttaggtcaagatgttatatagatgatggtgtaagtgatatgctgtgggggctgaaattgtgaggattagagtcaaatggctgaggtttgaggaacatgttcctgggcaaacccagctcccagcctctatcccctcaacccccacccacacaagcacctcacatcttggtttgttcagtcgacagttcattttattgatgcccactccaggctgcatagggttgaggggtgtacaagaggagaaccagattcagtccatgcctggaggttagtctggggggccggcgggatggacacacagacagacacatagatctggcatctgatagcagggcatacagtctcatgtaggggcactctgggcacaggtagtcaggaaggacttcctagagaagtcctcacagggggtgggggagcaaggagcaaggcagccatcagaaccttcgccttttgtacatttgtaggcaggtgacttttgagaccagtggaaccagggtgagcacaaagggctggggtctgggaatggcaggcagcaagggcttccctctcgcctcgtcatcccatagtggggaagccatctcccccacccccaattcttggcctgtactttgccttctgccacatgggccctttcctggcatccacctccgtggttttcaaggcttccctgagacaatccacgtggaccaggtgctctacccagtgcctggcacctcatcaccaccccatcaatgcacattcttgtcctaagtgctatctacggactatctagatcttggttccagctctgagtggtgtcagcggtcatgacaccccaggtcagggcccctagcaaccgcagagctcccaagagccctaggtaccttcactgcactgtgaaggcagagcttggtcccagcctacctccagggctggccagctctgcactgccttcctctcactgctcagtggcccatggacacccacttcccttccagtggcatgtgggcatccacttctcagatatgcagccctgaagaagagaccctacagctcttagggccaacactacccagcactgtaagcacgagcagaaaccagcctcagcccgtagcccagggaccccctttctttcactagtacaaagtttctgtggttggggaaccagcttcatgtcgggctaggcactctggcctctgatgtctgcctgctctggggttgcttccccatacctttggggcagcagggaggaggcctgcagcagacccctcaatctccaggcagaagcaggtgcaaaaacaaatatattctggggctgggttgggactcaccccttggacctccttaataggccatgaatcccaacgacagagagtcttgatgttgaaaaactatttctgaaggtgggtgggggtttgtaagtgcaacccacagatggggctgggacagggaaaggggactcctggagtcggtcctcaccacggccacagccctgaacgtagagcaggtaggagcaggtgggggcaggtggaggcaggcgaggccctggcagtcattctcgacaggagggcagcagtgggggaggtgcatgtagcttcaggccccctcagtcagtctggcctcagacttctaagggctagaagaggtatttgcggcaggactcagtcccacacttgcattcaatacggacccgcttcttaggggagccagggagcccagccaggccaaagttggagtccatgcgggtgctctccatgtccacggggtccactgtggagtcacagccagggaggagaggagggaaggaggcagggagggagggggagaaggagggagggaggaaggtagggaaggaggcaggggacttgttagaaggcctggatgaagggtcccactaccctttataccatgagaccaaggatctttggggctgggagcaactctgggatttgagttgggagatcttagggttggtggtttggggctagggccagggctcagcactggaggttgttcaggccaggggttgaagaatgctgggggaggcagggagccgtcaaggaaccaggagggtcgttttaggtgtgtgggggggggcaggggcaggaatagagggccacctgggcatccagaggtacatctgaactctcctgccaaagacctgcccttgagggtctcagaggtggctagcttatcaaggaggggactggctggaggccacaagggtgtccagaggcttgggatgaagaaggccaaagggagctaggagtggggaggggctgggggatgagggaaactcagtcaagactggagtcaggagtttcttaactctccaggaatcttgggagaagttggtatgaatgcctcagggttagggttcaggggctgggaatgaggggactcgaggggtaggcaggggtctagtagtccgaatgggggactgggagtcaggaagagcatcttaaagctcccgctaaagaattttccttggttctctgagtgactggtgtgcctttgggaggggtccttgtgtcccaagtcacagtgcgcccctgcccccagtcccttgccacccccaccttgcatgttgtaatcaaaggtgagctcctcgcctgcccggatggttcttgtggcaaagaaagcgatgcggggcagccgctcgtcaaggttgtctatgaagacgttgtacacctgcaggttggggtcacactgggcgagaaagaacagttgggagagtgaggaggggctccagcctgcccctctcgaccccttgcccgcctgccgggggtacccacactgtggttgacaaagtgggagatgttgccatagtaggcggcatccacggtgtacacgtcctccacgtagtccaggtcaaagaggtaggtggcgccctgacggtcgtagatctggccccgccgctctgcctcctctgaggtaatgatctagggaaagggccatgggtaaggggagattggccgcgaagtctcaggaaagccggcactgctcctgacttctgaaggacagggagcaggaggcacccctgcccgtggacacacatgggcagtgacgcagagagtctgtcggcccttcctgaggagctgggcctcagctggcaactgcctggcccgcctcaggcctctggctagcaggaggagtccctggcggtgagtgcaggctggcctggctgcagggcccaactggggccacgcgagcacctcctgccgccacccagcaagagctgcccaagaacccctggagcctccagtgccttgggacggagcaggaaggtgatcaggagagaccaaagcaagggcaagaaagaaagtgacccagggtgccacagggagagatacttactgatccaggcaagggagcctgagcccatgcaggacccagcagggagggcgtggagagaggggaagaagagacccaggccatggggaaagggggcaagggcctcaagacaagagaggtggaggcagaggcagagggaggcccaggttcagagagccagaaagaggctgaggccacacacttcagagacagcagcagaggaggaggaaagatgaagggagagcacagtgagagaagctctccctcggcccagggctgcgcagtgccaactggtgcttcagacggcagtgggtgcctcctccccagctggttccctacctgtcctaggaaatgggaagctttgctgccatcctagccccagcctgtccttgcctcccaatccacgccaacaaatctccggccccatggcccctcttccctgaggccagcaacggcagcctcctgccaatccccaagcgtagctgaccaggcccttccctgggtaggggtcccactgtacctcagccaagaacagtgcacagcacacaggagacacatggggactgctgtttactttgattctgcttcctggcttagcagcctgtcctctgtccaagtggcggccctccaggtagctccagtactcccccgccacacttcttccttcccctcccttctccagagacccctctcgggcttccttccttgctcaccctcatgtgggccggtgaccatgcccttctgacttgggaatccatctccttagctaggatggccacactggtgttctccctggctgtcccctcaaggctgtgttccatgccaaaggctgactaacaatgccttgaggaaaccaagatttaccctgcctcgggccacactacccactgccccgtgctccagttctgttccttgaggaacctggctcccttgccagggtttttattgaggccttccttgtgcccagtcccagcatgcagggctcacggtctgatcagggagacacagggaccaggcccggtccactctcaggggtcacaggtgggtaggggatacacaggaaccaggattggtccctgccctcaaggggtcaaagtctggtcacagagagacagattcacacacccacagtgtaagactgtgatcaggaaacagtgcttagggcttgagggggaatggagaagagacggattctctaaaacgagttgcagatcataaaagggctttaccacctggtggaagagtggggactctctcctaagggccacaacatgagggtggaatatgtcaagatcagtctcccttcacctcctggagaacagtcttccttgtggcatcagctcaaacagatgggacctctctaggcagccttccaagcttcccgttcctcccctacctcagaagctggccacttgtctgatcaagtcctgggatcaaagggcagcctctcccaggctcacaggcctagtgggtggggtatgagccatctttttcaccgtaggccctccttgagccaggaggagtgcccacatccttcctagtctccatcttcatgcctattctgccactacctgctctcccagtctcaccaggagtgagtcatactgcaggccctccagggcacaaacacttcttggttccttctcatcttcttttcagtcccattctctaatgattggtcaaccctgaaagcttgggaacagatgggcctgtctctgctccctaacagctctccagaatgaatggaaaagacaggctgtaagctggcagttccgagagggtgctgacgtgaatgggaactagaagacttggagtcagctgctgctctgccccctcctggatatgggatcctgaatgttctctctgtgcctgtttcctcagctaaaaaaaaaaaaaaaaagctacagcctggcagggtaccagttttagatattatttgggcctttatttttttatccattgtactacttttgtgattttaatcaagttattctacctctctgggcctcagttttctcatctgcagaatgggaatgacagtgacaaaatcccaacctgacaggggctgttgtaaagatccatacaagatcatggacgtgaaaggtgttcctgtttctgataggagagtgagcaatgttaggagtggttatgaggaccagttataaatgtaaggcacacccttaattttatgtgccctttaccaagaaaggaaaaactgctgttaattataaatgatacaccatgatttaattaagacatcttcatttccgagatgttaaaataggaagaaaatgcatgttttagaatggataaaataggctggcattgtctcttgggtgaggatgggaatgttctgcaggaacgtgaaagttctgacccaatgtgggaggctgcaaaggagggaagggcagctcccgtggagggagctcttggtacagactgtcagtacatgtcccagctgaagaaggacagtagatagcatggtgtgcagataggcccttgattccgtaacaccctggcagcatctgggacggcactacagaatcaagtaaattagaactgctgcaacgaaatgtatgactattaccccaagtaggataaataaaaactacaaataggaccctatccggtcaggctgggtcctgtagccagatgggttacaaaaaaacttggtcttccagacattttggatttgctgaagtttgaactatgctgataacatttgtatcaaacttactgtgtactgaacactgttcaagtgctttttccaagtgcttgatacatagtaactcatttaatttttgtaacaagcctgtgaggtgagtgctattattgttcccattttacatagtaaatgcaggcacagaggttagtgattgctgatgagtagtggagctgggattcagcctcgggcagtcaggatccagagtccaccctcctaaccatttgctatactgtctcttcagaggggaatgcgggaagatgagtttggctctttccggaggcctgtggcaccatcaggaggacccatgggtctgagggtgctggagggcggtcaggaaatagtgcagcaagaatgtgtggagagcaggaagggtgaggacagggctgagaggatccctgggaggattaaagacactgacactaaggaagagcagccagaaaagtctcaggcccaggagggtctgaggctgcccttggggtgtagtcttcctcctcgactgtcaggaccctgtgtggtagacagtgtttcctccagatgccagtacagtgaggctgagatacttcctgctctgtgcagcccctaggacggtggactgtctgggtcctatggggtctggacatctcctccagagagtttgggttccttcaacctctggacacttcctccagagcgtcaggacattgagggcatggacacttcccccgatactgtcagggtgcccttgggtgtagactagatctccaggctgtccggatccctctggggtgaggacacttctttttcctgtcatgacaacatgggtgtgaacacttcctaagtcagaccaccaggtcctctagagtgtggagagcacaagctcttcctccaggtcactggggtcactgtgggacatggacttcctgcagattatcaggactccttcccccgcccactgtcaggagccctcagggtgtggacacctccccacctcctccttccagatggccaggtcctgtggcatgtagacatctcctccagcaggccagcagggttatctggacagggtgtggctgtctccttgcccagactgtcagggtcctctgtggtgtagattgttctacccaagattgtctctgtccctgtcagatgtgaccctctgtacagtttctcagagtaggagcctctccttgtcatcagtatcagtgctacagtgtggacaccctcatgtgagtgttcctcaggccagtttctgtcactttccaactatatcacacccctaagggacctatggctatatatagaggtatctggggttgtcactatgacagggcacactcatggatttaaatggtcaaggtccagggatgctaaatgtcccccaatgcatgagacagttctacataacgaaaaatcatctcatcccaaatgctaactgcatcccatcttagaatgcgacatttttctgttgtagatacttgctagttatctagttgctagttatggctgtgctttagccttgtgcagctgggcacatggctaaagcacatcctgttgcctctcttgcatggaggtaaggccagtgactcagttccagccagtggagcatgggcagaagtgatgggtgctgcatctaggcctggcccactaggatgggccccttctatcccccacttggcccgcctgccaggcaatgctgaggatctggtagtgactccaaggcttcaaggcacagcaaaggcacaggagggaaagagcctaggtgtcccagttgctgtgtgaaaggatacccactcaaacccccacaatggactgttccatgagcaagcaaacttgtaatgtgcaaagtcactaagatgagggggttgtttttacaatggctggcttgcctaatacactcggccttccccagtgtcctgaaagtctaaaggagttagccaacctttgtccaaggcatgtctcatcctactccagactgcgttccttctgtgcagactggtaaaacaatgctcctcaggcagatactgcttggtgataagatgggcccttgtgaaatgagaagaggaagtgcctttccagctgatgcagccctatgccagaccacatggcctgttacaaggaatgctaaggctgcctggatctcagctcccacttttcttctttgctaagtaccctgataaagtcaacccagtgcagaagtattttggtgttcctgtaaccaccagcatggccatgctggtgtgtaccagcttggaatactagcccctgcccaatgtccctgtgacactcagatcagtctcccaggcctttcccatagggaaagcagggagtttcagacacacagtgaggagagccgggtgcacactgtgaacacagggtgggaagagctgcacacacacaccgggtcccatctccctacctctcccacgtactccatgacgaagctgttcttgcgaatcttctccagggtgcggacgccccagccacgcccatcatccgtgcggaagatgcagaggtcatatcggatacccttctgtaccacacgatttgggcagtcatagccgcagcggcagcgggagttgcactcgtagatgggcagcccggctcgaagccgcacctggccctggtcattgtaggcaaacttgtgcagtgacgcccccgggcagcagcctccagtgggtgcccacagacagtcctggcactcgcagcccacagccacctggttgagggtgatgccctcaccaacacggtactcattgatgtacacgaaggcccgcggagggccgtccaggtccacctcattctctacagtgatgcgtcccagatggctgcgcttggcattgagctcctgctcccagcgacggagcgcccgcctctgcttggccttctgcaccaggtagttggccaagcttgggtccaggtgccggggggtctttgaccggtggtgccgccggagcagctccctttctaagtccttgtggaactgcttgaggatacgcacacacttgagattctgccgtggctcccaggtgctctctgagtctggatatccacgccatttcaccaggtaatattcctgttcctgttgggggtgggtagggggacgggggtaccgtaagtagtgagatctttgggccagccttcatggggcttccatcccaaagctgatggataacacctaccccaccccaccccttttctggaatgacttcatgcggtgattaaaaagcatgggctctagagtgaggattactaggttcaaatcctcctctcccccttactgtctgtccaaactgttccaacttttaattaatctcccaaacccactcctctccttggtcttccccatcatcagctccctcccttctagctgttcagaccggaactcctggagtcatccttgacttctcttttgttcacacttccgacacccaccctctgcacatcccgttggccccaccttcaaaatacgtccagtgacaaaccatcatcactgccaccaccctggctcacctggaccctgacaagtccttgcctgttctaggcagcttacatgtattattaactaacttaatcctcacaaccccacatgaggtagatgctcctagttatccctcttttacgacaaggaaaatgaagtactgaaggtttgggtaggtagcctggggtaacacaaccaggactggaacccaagcatgctgactccagtttgttctcctaaccatcctgccactcccccttctggcaataactatccccatttcagttgttaggggattaaatgagtttccgagaggaaacgcctggcacactgaaagtgctcaatagccattagctattgacatttccactccagggttcagcatctcaatctctgcaattactttgtattcctagggagatgggaagagatttatttgggaattcattccggctcctgcccagaaagctacttagtttccctatctgtaacatagggataaaagggtctattcatcctgtgttttcagggtcaaaggagaaaattccctttggaaacagatgtgggcagttggggacaagagggcaggacactaacttccttgtgacctgtcccctcccagagcatggtcaccccagactcacgcggatcttcttgtaatcgcacaggtactcgacttcaaagtcatagaggttcctcttagagataccgagggcagggcaggagagcttggccaggcggcacaggtcctgcagctgattccaagaagacttgcaaca

SEQ ID NO: 6 - DNA, 925 bp, Homo sapiens
  misc_feature 1..925: note = AF196970.3 cDNA sequence
  source 1..925: mol_type = other DNA
cgaggcagggctttggctactggagatcgtaggttcgaatcccgtctgggaagttcaacttgtgcacctgtaaaagaagctggcattattggcttgtactcaagggctggcacagagtgtgtcggggtgcggacgccccagccacgcccatcatccgtgcggaagatgcagaggtcatatcggatacccttctgtaccacacgatttgggcagtcatagccgcagcggcagcgggagttgcactcgtagatgggcagcccggctcgaagccgcacctggccctggtcattgtaggcaaacttgtgcagtgacgcccccgggcagcagcctccagtgggtgcccacagacagtcctggcactcgcagcccacagccacctggttgagggtgatgccctcaccaacacggtactcattgatgtacacgaaggcccgcggagggccgtccaggtccacctcattctctacagtgatgcgtcccagatggctgcgcttggcattgagctcctgctcccagcgacggagcgcccgcctctgcttggccttctgcaccaggtagttggccaagcttgggtccaggtgccggggggtctttgaccggtggtgccgccggagcagctccctttctaagtccttgtggaactgcttgaggatacgcacacacttgagattctgccgtggctcccaggtgctctctgagtctggatatccacgccatttcaccagggtcaaaggagaaaattccctttggaaacagatgtgggcagttggggacaagagggcaggacactaacttccttgtgacctgtcccctcccagagcatggtcaccccagactcacgcggatcttcttgtaatcgcacaggtactcgacttcaaagtcatagaggttcctcttagagataccgagggcagggcaggagagc

SEQ ID NO: 7 - DNA, 125 bp, Homo sapiens
  misc_feature 1..125: note = AF196970.3 exon 1 cDNA sequence
  source 1..125: mol_type = other DNA
cgaggcagggctttggctactggagatcgtaggttcgaatcccgtctgggaagttcaacttgtgcacctgtaaaagaagctggcattattggcttgtactcaagggctggcacagagtgtgtcgg

SEQ ID NO: 8 - DNA, 600 bp, Homo sapiens
  misc_feature 1..600: note = AF196970.3 exon 2 cDNA sequence
  source 1..600: mol_type = other DNA
ggtgcggacgccccagccacgcccatcatccgtgcggaagatgcagaggtcatatcggatacccttctgtaccacacgatttgggcagtcatagccgcagcggcagcgggagttgcactcgtagatgggcagcccggctcgaagccgcacctggccctggtcattgtaggcaaacttgtgcagtgacgcccccgggcagcagcctccagtgggtgcccacagacagtcctggcactcgcagcccacagccacctggttgagggtgatgccctcaccaacacggtactcattgatgtacacgaaggcccgcggagggccgtccaggtccacctcattctctacagtgatgcgtcccagatggctgcgcttggcattgagctcctgctcccagcgacggagcgcccgcctctgcttggccttctgcaccaggtagttggccaagcttgggtccaggtgccggggggtctttgaccggtggtgccgccggagcagctccctttctaagtccttgtggaactgcttgaggatacgcacacacttgagattctgccgtggctcccaggtgctctctgagtctggatatccacgccatttcaccag

SEQ ID NO: 9 - DNA, 200 bp, Homo sapiens
  misc_feature 1..200: note = AF196970.3 exon 3 cDNA sequence
  source 1..200: mol_type = other DNA
ggtcaaaggagaaaattccctttggaaacagatgtgggcagttggggacaagagggcaggacactaacttccttgtgacctgtcccctcccagagcatggtcaccccagactcacgcggatcttcttgtaatcgcacaggtactcgacttcaaagtcatagaggttcctcttagagataccgagggcagggcaggagagc

SEQ ID NO: 10 - protein, 164 aa, Homo sapiens
  DOMAIN 22..164: note = Intracellular signal
  SITE 61..89: note = ITAM1
  SITE 100..128: note = ITAM2
  SITE 131..159: note = ITAM3
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 11 - protein, 645 aa, Homo sapiens
  DOMAIN 1..42: note = Extracellular domain
MPIWKFPDEEGACQPCPINCTHSCVDLDDKGCPAEQRASPLTSIVSAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERAKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 12 - protein, 42 aa, Homo sapiens
MPIWKFPDEEGACQPCPINCTHSCVDLDDKGCPAEQRASPLT

SEQ ID NO: 13 - RNA, 26 nt, synthetic construct
  source 1..26: mol_type = other RNA
gttgcaagtcttcttggaatcagctg

SEQ ID NO: 14 - RNA, 21 nt, synthetic construct
  source 1..21: mol_type = other RNA
agtcgagtacctgtgcgatta

SEQ ID NO: 15 - RNA, 21 nt, synthetic construct
  source 1..21: mol_type = other RNA
agatccgcgaacaggaatatt

SEQ ID NO: 16 - RNA, 21 nt, synthetic construct
  source 1..21: mol_type = other RNA
gatccgcgaacaggaatatta

SEQ ID NO: 17 - RNA, 21 nt, synthetic construct
  source 1..21: mol_type = other RNA
gcagttccacaaggacttaga

SEQ ID NO: 18 - RNA, 21 nt, synthetic construct
  source 1..21: mol_type = other RNA
cctaaggttaagtcgccctcg

SEQ ID NO: 19 - RNA, 10 nt, synthetic construct
  source 1..10: mol_type = other RNA
ttgatatccg

SEQ ID NO: 20 - RNA, 26 nt, synthetic construct
  source 1..26: mol_type = other RNA
cagctgattccaagaagacttgcaac

SEQ ID NO: 21 - RNA, 21 nt, synthetic construct
  source 1..21: mol_type = other RNA
taatcgcacaggtactcgact

SEQ ID NO: 22 - RNA, 21 nt, synthetic construct
  source 1..21: mol_type = other RNA
aatattcctgttcgcggatct

SEQ ID NO: 23 - RNA, 21 nt, synthetic construct
  source 1..21: mol_type = other RNA
taatattcctgttcgcggatc

SEQ ID NO: 24 - RNA, 21 nt, synthetic construct
  source 1..21: mol_type = other RNA
tctaagtccttgtggaactgc

SEQ ID NO: 25 - RNA, 19 nt, synthetic construct
  source 1..19: mol_type = other RNA
agggcgacttaaccttagg

SEQ ID NO: 26 - RNA, 62 nt, synthetic construct
  source 1..62: mol_type = other RNA
gttgcaagtcttcttggaatcagctgttgatatccgcagctgattccaagaagacttgcaac

SEQ ID NO: 27 - RNA, 48 nt, synthetic construct
  source 1..48: mol_type = other RNA
agtcgagtacctgtgcgattactcgagtaatcgcacaggtactcgact

SEQ ID NO: 28 - RNA, 48 nt, synthetic construct
  source 1..48: mol_type = other RNA
agatccgcgaacaggaatattctcgagaatattcctgttcgcggatct

SEQ ID NO: 29 - RNA, 48 nt, synthetic construct
  source 1..48: mol_type = other RNA
gatccgcgaacaggaatattactcgagtaatattcctgttcgcggatc

SEQ ID NO: 30 - RNA, 49 nt, synthetic construct
  source 1..49: mol_type = other RNA
gcagttccacaaggacttagatcaagagtctaagtccttgtggaactgc

SEQ ID NO: 31 - RNA, 46 nt, synthetic construct
  source 1..46: mol_type = other RNA
cctaaggttaagtcgccctcgcgagcgagggcgacttaaccttagg

SEQ ID NO: 32 - DNA, 26 bp, synthetic construct
  source 1..26: mol_type = other DNA
gttgcaagtcttcttggaatcagctg

SEQ ID NO: 33 - DNA, 21 bp, synthetic construct
  source 1..21: mol_type = other DNA
agtcgagtacctgtgcgatta

SEQ ID NO: 34 - DNA, 21 bp, synthetic construct
  source 1..21: mol_type = other DNA
agatccgcgaacaggaatatt

SEQ ID NO: 35 - DNA, 21 bp, synthetic construct
  source 1..21: mol_type = other DNA
gatccgcgaacaggaatatta

SEQ ID NO: 36 - DNA, 21 bp, synthetic construct
  source 1..21: mol_type = other DNA
gcagttccacaaggacttaga

SEQ ID NO: 37 - DNA, 21 bp, synthetic construct
  source 1..21: mol_type = other DNA
cctaaggttaagtcgccctcg

SEQ ID NO: 38 - DNA, 10 bp, synthetic construct
  source 1..10: mol_type = other DNA
ttgatatccg

SEQ ID NO: 39 - DNA, 26 bp, synthetic construct
  source 1..26: mol_type = other DNA
cagctgattccaagaagacttgcaac

SEQ ID NO: 40 - DNA, 21 bp, synthetic construct
  source 1..21: mol_type = other DNA
taatcgcacaggtactcgact

SEQ ID NO: 41 - DNA, 21 bp, synthetic construct
  source 1..21: mol_type = other DNA
aatattcctgttcgcggatct

SEQ ID NO: 42 - DNA, 21 bp, synthetic construct
  source 1..21: mol_type = other DNA
taatattcctgttcgcggatc

SEQ ID NO: 43 - DNA, 21 bp, synthetic construct
  source 1..21: mol_type = other DNA
tctaagtccttgtggaactgc

SEQ ID NO: 44 - DNA, 19 bp, synthetic construct
  source 1..19: mol_type = other DNA
agggcgacttaaccttagg

SEQ ID NO: 45 - DNA, 62 bp, synthetic construct
  source 1..62: mol_type = other DNA
gttgcaagtcttcttggaatcagctgttgatatccgcagctgattccaagaagacttgcaac

SEQ ID NO: 46 - DNA, 48 bp, synthetic construct
  source 1..48: mol_type = other DNA
agtcgagtacctgtgcgattactcgagtaatcgcacaggtactcgact

SEQ ID NO: 47 - DNA, 48 bp, synthetic construct
  source 1..48: mol_type = other DNA
agatccgcgaacaggaatattctcgagaatattcctgttcgcggatct

SEQ ID NO: 48 - DNA, 48 bp, synthetic construct
  source 1..48: mol_type = other DNA
gatccgcgaacaggaatattactcgagtaatattcctgttcgcggatc

SEQ ID NO: 49 - DNA, 49 bp, synthetic construct
  source 1..49: mol_type = other DNA
gcagttccacaaggacttagatcaagagtctaagtccttgtggaactgc

SEQ ID NO: 50 - DNA, 46 bp, synthetic construct
  source 1..46: mol_type = other DNA
cctaaggttaagtcgccctcgcgagcgagggcgacttaaccttagg